SEQUENCE LISTING (107 sequences)

SEQ ID NO: 1 - protein, 11 aa, Artificial Sequence
  Synthetic: CDR-L1 of rat1B2, hu1B2.L1, hu1B2.L7, hu1B2.v101, hu1B2.v102, hu1B2.v103, hu1B2.v104
QTSEDIYSGLA

SEQ ID NO: 2 - protein, 7 aa, Artificial Sequence
  Synthetic: CDR-L2 of rat1B2, hu1B2.L1, hu1B2.L7, hu1B2.v101, hu1B2.v102, hu1B2.v103, hu1B2.v104
GASRLQD

SEQ ID NO: 3 - protein, 9 aa, Artificial Sequence
  Synthetic: CDR-L3 of rat1B2, hu1B2.L1, hu1B2.L7, hu1B2.v101, hu1B2.v102, hu1B2.v103, hu1B2.v104
QQGFKYPLT

SEQ ID NO: 4 - protein, 5 aa, Artificial Sequence
  Synthetic: CDR-H1 of rat1B2, hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104
DFYME

SEQ ID NO: 5 - protein, 19 aa, Artificial Sequence
  Synthetic: CDR-H2 of rat1B2
ASRNKANNFSIVYSASVKD

SEQ ID NO: 6 - protein, 19 aa, Artificial Sequence
  Synthetic: CDR-H2 of hu.1B2.v1.DFS.H1, hu.1B2.H10, hu.1B2.DFS.H14
ASRNKANDFSIVYSASVKD

SEQ ID NO: 7 - protein, 19 aa, Artificial Sequence
  Synthetic: CDR-H2 of hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104
AQRNKANDFSIVYSASVKD

SEQ ID NO: 8 - protein, 9 aa, Artificial Sequence
  Synthetic: CDR-H3 of rat1B2, hu.1B2.v1.DFS.H1, hu.1B2.H10, hu.1B2.DFS.H14, hu.1B2.H1.N54D.S51Q
DSGRWGLDA

SEQ ID NO: 9 - protein, 9 aa, Artificial Sequence
  Synthetic: CDR-H3 of hu.1B2.v2, hu1B2.v101
DGGRWGLDA

SEQ ID NO: 10 - protein, 9 aa, Artificial Sequence
  Synthetic: CDR-H3 of hu.1B2.v4, hu1B2.v102
DGGRLALDA

SEQ ID NO: 11 - protein, 9 aa, Artificial Sequence
  Synthetic: CDR-H3 of hu.1B2.v8, hu1B2.v104
DGGKLALDA

SEQ ID NO: 12 - protein, 9 aa, Artificial Sequence
  Synthetic: CDR-H3 of hu.1B2.v9, hu1B2.v103
DGGKWGLDA

SEQ ID NO: 13 - protein, 107 aa, Artificial Sequence
  Synthetic: rat.1B2 Light Chain Variable Region (VL)
DIQMTQSPASLSASLGETVTIQCQTSEDIYSGLAWYHQKPGKSPQLLIYGASRLQDGVPSRFTGSGSGTQYSLKISSMQTEDEGVYFCQQGFKYPLTFGSGTKLEIK

SEQ ID NO: 14 - protein, 120 aa, Artificial Sequence
  Synthetic: rat.1B2 Heavy Chain Variable Region (VH)
EVKLVDYGGGLVQPGASLRLSCEVSGFTFSDFYMEWIRQAPGKGLEWIAASRNKANNFSIVYSASVKDRFTISRDTYKSILYLQMSTLKPEDTAVYYCSRDSGRWGLDAWGQGTSVIVSS

SEQ ID NO: 15 - protein, 107 aa, Artificial Sequence
  Synthetic: hu.1B2.L1 VL
DIQMTQSPSSVSASVGDRVTITCQTSEDIYSGLAWYQQKPGKSPKLLIYGASRLQDGVPSRFSGSGSGTDYTLTISSLQPEDFATYFCQQGFKYPLTFGGGTKVEIK

SEQ ID NO: 16 - protein, 107 aa, Artificial Sequence
  Synthetic: hu.1B2.L7 VL
DIQMTQSPSSVSASVGDRVTITCQTSEDIYSGLAWYQQKPGKSPKLLIYGASRLQDGVPSRFSGSGSGTDYTLTISSLQPEDFATYYCQQGFKYPLTFGGGTKVEIK

SEQ ID NO: 17 - protein, 120 aa, Artificial Sequence
  Synthetic: hu.1B2.v1.DFS.H1 VH
EVQLVESGGGLVQPGGSLKLSCAVSGFTFSDFYMEWIRQASGKGLEWIAASRNKANDFSIVYSASVKDRFTISRDTSKSTLYLQMNSLKTEDTAVYYCSRDSGRWGLDAWGQGTLVTVSS

SEQ ID NO: 18 - protein, 120 aa, Artificial Sequence
  Synthetic: hu.1B2.H10 VH
EVQLVESGGGLVQPGGSLKLSCAVSGFTFSDFYMEWVRQASGKGLEWVAASRNKANDFSIVYSASVKDRFTISRDDSKSTAYLQMNSLKTEDTAVYYCSRDSGRWGLDAWGQGTLVTVSS

SEQ ID NO: 19 - protein, 120 aa, Artificial Sequence
  Synthetic: hu.1B2.DFS.H14 VH
EVQLVESGGGLVQPGGSLKLSCAVSGFTFSDFYMEWVRQASGKGLEWVAASRNKANDFSIVYSASVKDRFTISRDDSKSTLYLQMNSLKTEDTAVYYCSRDSGRWGLDAWGQGTLVTVSS

SEQ ID NO: 20 - protein, 120 aa, Artificial Sequence
  Synthetic: hu.1B2.H1.N54D.S51Q VH
EVQLVESGGGLVQPGGSLKLSCAVSGFTFSDFYMEWIRQASGKGLEWIAAQRNKANDFSIVYSASVKDRFTISRDTSKSTLYLQMNSLKTEDTAVYYCSRDSGRWGLDAWGQGTLVTVSS

SEQ ID NO: 21 - protein, 120 aa, Artificial Sequence
  Synthetic: hu.1B2.v2 VH
EVQLVESGGGLVQPGGSLKLSCAVSGFTFSDFYMEWIRQASGKGLEWIAAQRNKANDFSIVYSASVKDRFTISRDTSKSTLYLQMNSLKTEDTAVYYCSRDGGRWGLDAWGQGTLVTVSS

SEQ ID NO: 22 - protein, 120 aa, Artificial Sequence
  Synthetic: hu.1B2.v4 VH
EVQLVESGGGLVQPGGSLKLSCAVSGFTFSDFYMEWIRQASGKGLEWIAAQRNKANDFSIVYSASVKDRFTISRDTSKSTLYLQMNSLKTEDTAVYYCSRDGGRLALDAWGQGTLVTVSS

SEQ ID NO: 23 - protein, 120 aa, Artificial Sequence
  Synthetic: hu.1B2.v8 VH
EVQLVESGGGLVQPGGSLKLSCAVSGFTFSDFYMEWIRQASGKGLEWIAAQRNKANDFSIVYSASVKDRFTISRDTSKSTLYLQMNSLKTEDTAVYYCSRDGGKLALDAWGQGTLVTVSS

SEQ ID NO: 24 - protein, 120 aa, Artificial Sequence
  Synthetic: hu.1B2.v9 VH
EVQLVESGGGLVQPGGSLKLSCAVSGFTFSDFYMEWIRQASGKGLEWIAAQRNKANDFSIVYSASVKDRFTISRDTSKSTLYLQMNSLKTEDTAVYYCSRDGGKWGLDAWGQGTLVTVSS

SEQ ID NO: 25 - protein, 107 aa, Artificial Sequence; identical to SEQ ID NO: 16
  Synthetic: hu.1B2.v101 VL

SEQ ID NO: 26 - protein, 120 aa, Artificial Sequence
  Synthetic: hu.1B2.v101 VH
EVQLVESGGGLVQPGGSLKLSCAVSGFTFSDFYMEWVRQASGKGLEWVAAQRNKANDFSIVYSASVKDRFTISRDDSKSTLYLQMNSLKTEDTAVYYCSRDGGRWGLDAWGQGTLVTVSS

SEQ ID NO: 27 - protein, 107 aa, Artificial Sequence; identical to SEQ ID NO: 16
  Synthetic: hu.1B2.v102 VL

SEQ ID NO: 28 - protein, 120 aa, Artificial Sequence
  Synthetic: hu.1B2.v102 VH
EVQLVESGGGLVQPGGSLKLSCAVSGFTFSDFYMEWVRQASGKGLEWVAAQRNKANDFSIVYSASVKDRFTISRDDSKSTLYLQMNSLKTEDTAVYYCSRDGGRLALDAWGQGTLVTVSS

SEQ ID NO: 29 - protein, 107 aa, Artificial Sequence; identical to SEQ ID NO: 16
  Synthetic: hu.1B2.v103 VL

SEQ ID NO: 30 - protein, 120 aa, Artificial Sequence
  Synthetic: hu.1B2.v103 VH
EVQLVESGGGLVQPGGSLKLSCAVSGFTFSDFYMEWVRQASGKGLEWVAAQRNKANDFSIVYSASVKDRFTISRDDSKSTLYLQMNSLKTEDTAVYYCSRDGGKWGLDAWGQGTLVTVSS

SEQ ID NO: 31 - protein, 107 aa, Artificial Sequence; identical to SEQ ID NO: 16
  Synthetic: hu.1B2.v104 VL

SEQ ID NO: 32 - protein, 120 aa, Artificial Sequence
  Synthetic: hu.1B2.v104 VH
EVQLVESGGGLVQPGGSLKLSCAVSGFTFSDFYMEWVRQASGKGLEWVAAQRNKANDFSIVYSASVKDRFTISRDDSKSTLYLQMNSLKTEDTAVYYCSRDGGKLALDAWGQGTLVTVSS

SEQ ID NO: 33 - protein, 16 aa, Artificial Sequence
  Synthetic: rat.3107 CDR-L1, hu.3107.L1, hu.3107.L6, and hu.3107.L7
RSSQSLVHSDGNTYLH

SEQ ID NO: 34 - protein, 7 aa, Artificial Sequence
  Synthetic: rat.3107 CDR-L2, hu.3107.L1, hu.3107.L6, and hu.3107.L7
RISNRFS

SEQ ID NO: 35 - protein, 9 aa, Artificial Sequence
  Synthetic: rat.3107 CDR-L3, hu.3107.L1, hu.3107.L6, and hu.3107.L7
LQSTHFPDT

SEQ ID NO: 36 - protein, 5 aa, Artificial Sequence
  Synthetic: rat.3107 CDR-H1, hu.3107.V1-2.H1, hu.3107.H12, hu.3107.H13, hu.3107.V5-51.H1, hu.3107.H14, and hu.3107.H15
NYVIH

SEQ ID NO: 37 - protein, 17 aa, Artificial Sequence
  Synthetic: rat.3107 CDR-H2, hu.3107.V1-2.H1, hu.3107.H12, hu.3107.H13, hu.3107.V5-51.H1, hu.3107.H14, and hu.3107.H15
YIIPGSGGTKFNEKFKG

SEQ ID NO: 38 - protein, 8 aa, Artificial Sequence
  Synthetic: rat.3107 CDR-H3, hu.3107.V1-2.H1, hu.3107.H12, hu.3107.H13, hu.3107.V5-51.H1, hu.3107.H14, and hu.3107.H15
DGAGSFTY

SEQ ID NO: 39 - protein, 112 aa, Artificial Sequence
  Synthetic: rat.3107 VL
DVLMTQTPVSLPVSLGGQVSISCRSSQSLVHSDGNTYLHWFLQKPGQSPQLLIYRISNRFSGVPDRFSGSGSGTDFTLKISRVEPEDLGVYYCLQSTHFPDTFGGGTKVEIK

SEQ ID NO: 40 - protein, 117 aa, Artificial Sequence
  Synthetic: rat.3107 VH
QVQLQQSGAELAKSGSSVKISCKASGYTFSNYVIHWIKQTTGQAPEWTGYIIPGSGGTKFNEKFKGKATLTVDKSSTTAYMQLSSLTPVDTAVYYCARDGAGSFTYWGQGTLVTVSS

SEQ ID NO: 41 - protein, 11 aa, Artificial Sequence
  Synthetic: rb.2338 CDR-L1
QASQSISSYLA

SEQ ID NO: 42 - protein, 7 aa, Artificial Sequence
  Synthetic: rb.2338 CDR-L2
RASKLAS

SEQ ID NO: 43 - protein, 12 aa, Artificial Sequence
  Synthetic: rb.2338 CDR-L3
QSNSYGNNWVGG

SEQ ID NO: 44 - protein, 6 aa, Artificial Sequence
  Synthetic: rb.2338 CDR-H1
SGYDMC

SEQ ID NO: 45 - protein, 17 aa, Artificial Sequence
  Synthetic: rb.2338 CDR-H2
CIYAGSEGFTYYASWAK

SEQ ID NO: 46 - protein, 9 aa, Artificial Sequence
  Synthetic: rb.2338 CDR-H3
WTDSDGSNL

SEQ ID NO: 47 - protein, 110 aa, Artificial Sequence
  Synthetic: rb.2338 VL
DVVMTQTPASVEAAVGGTVTIKCQASQSISSYLAWYQQKPGQPPKLLIYRASKLASGVPSRFSGRGSGTQFTLTISDLECADAATYYCQSNSYGNNWVGGFGGGTKVEIK

SEQ ID NO: 48 - protein, 118 aa, Artificial Sequence
  Synthetic: rb.2338 VH
QSLEESGGDLVKPGASLTLTCTASGFSFSSGYDMCWVRQAPGKGLEWIACIYAGSEGFTYYASWAKGRFTISKSSSTTVTLQMTSLTVADTATYFCARWTDSDGSNLWGPGTLVTVSS

SEQ ID NO: 49 - protein, 12 aa, Artificial Sequence
  Synthetic: rb.2430, rb.2430.C95dS CDR-L1
QASQSVVNNRLA

SEQ ID NO: 50 - protein, 7 aa, Artificial Sequence
  Synthetic: rb.2430, rb.2430.C95dS CDR-L2
GASTLES

SEQ ID NO: 51 - protein, 13 aa, Artificial Sequence
  Synthetic: rb.2430 CDR-L3
QGEFLCSSGDCVA

SEQ ID NO: 52 - protein, 13 aa, Artificial Sequence
  Synthetic: rb.2430.C95dS CDR-L3
QGEFLCSSGDSVA

SEQ ID NO: 53 - protein, 5 aa, Artificial Sequence
  Synthetic: rb.2430, rb.2430.C95dS CDR-H1
SYDMS

SEQ ID NO: 54 - protein, 15 aa, Artificial Sequence
  Synthetic: rb.2430 , rb.2430.C95dS CDR-H2
IIQAGSNTLFYASWA

SEQ ID NO: 55 - protein, 12 aa, Artificial Sequence
  Synthetic: rb.2430, rb.2430.C95dS CDR-H3
GGVIFIIGHFNL

SEQ ID NO: 56 - protein, 112 aa, Artificial Sequence
  Synthetic: rb.2430 VL
AQVLTQTASSVSAAVGGTVTINCQASQSVVNNRLAWYQQKPGQPPKLLMYGASTLESGVSSRFKGSGSGTQFTLTISGVQCDDAATYYCQGEFLCSSGDCVAFGGGTKVEIK

SEQ ID NO: 57 - protein, 112 aa, Artificial Sequence
  Synthetic: rb.2430.C95dS VL
AQVLTQTASSVSAAVGGTVTINCQASQSVVNNRLAWYQQKPGQPPKLLMYGASTLESGVSSRFKGSGSGTQFTLTISGVQCDDAATYYCQGEFLCSSGDSVAFGGGTKVEIK

SEQ ID NO: 58 - protein, 118 aa, Artificial Sequence
  Synthetic: rb.2430 VH
QSVEESGGRLVTPGTPLTLTCTVSGFSLSSYDMSWVRQAPGKGLEWIGIIQAGSNTLFYASWAKGRFTISKTSTTVDLKITSPTTEDTATYFCARGGVIFIIGHFNLWGPGTLVTVSS

SEQ ID NO: 59 - protein, 11 aa, Artificial Sequence
  Synthetic: rb.2621 CDR-L1
QASESIGSYLA

SEQ ID NO: 60 - protein, 7 aa, Artificial Sequence
  Synthetic: rb.2621 CDR-L2
RASTLAS

SEQ ID NO: 61 - protein, 12 aa, Artificial Sequence
  Synthetic: rb.2621 CDR-L3
QQTYSGAGVDNL

SEQ ID NO: 62 - protein, 6 aa, Artificial Sequence
  Synthetic: rb.2621 CDR-H1
SGYDMC

SEQ ID NO: 63 - protein, 16 aa, Artificial Sequence
  Synthetic: rb.2621 CDR-H2
CIVTVSGNTYYASWAK

SEQ ID NO: 64 - protein, 12 aa, Artificial Sequence
  Synthetic: rb.2621 CDR-H3
DGGFTDTWYFHL

SEQ ID NO: 65 - protein, 110 aa, Artificial Sequence
  Synthetic: rb.2621 VL
AYDMTQTPASVEVAVGGTVTIKCQASESIGSYLAWYQQKPGQPPKLLIYRASTLASGVPSRFKGSGSGTEFTLTISGVQCDDAATYYCQQTYSGAGVDNLFGGGTKVEIK

SEQ ID NO: 66 - protein, 120 aa, Artificial Sequence
  Synthetic: rb.2621 VH
QSLEESGGGLVQPGASLTLTCTASGFSFSSGYDMCWVRQAPGKGLEWIACIVTVSGNTYYASWAKGRFTISKTSSTTVTLQMTSLTAADTATYFCARDGGFTDTWYFHLWGPGTLVTVSS

SEQ ID NO: 67 - protein, 605 aa, Artificial Sequence
  Synthetic: MBP-huNotch2 EGF6-10
AGSMGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNGENLYFQGSDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDEGNSHHHHHHHH

SEQ ID NO: 68 - protein, 526 aa, Artificial Sequence
  Synthetic: MBP-huNotch2 EGF7-9
AGSMGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNGENLYFQGSERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDAGNSHHHHHHHH

SEQ ID NO: 69 - protein, 37 aa, Artificial Sequence
  Synthetic: huNotch2 EGF7 (amino acids 260-296); cynoNotch2 EGF7 (amino acids 260-296)guinea pig Notch2 EGF7 (amino acids 244-280)
NIDDCPNHRCQNGGVCVDGVNTYNCRCPPQWTGQFCT

SEQ ID NO: 70 - protein, 2471 aa, Homo sapiens
  misc_feature (1)..(2471): Human Notch2, with signal sequence (amino acids 1-25)
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 71 - protein, 2471 aa, Macaca fascicularis
  misc_feature (1)..(2471): Cynomolgus monkey Notch2, with signal sequence (amino acids 1-25)
MPALRPALLWALLALWLCRAAPARALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQHCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCVDGVNTFSCLCLPGFTGDKCQTDMNECLSEPCKNGGTCSDYVNSYTCKCQAGFDGVHCENNIDECTESSCFNGGTCVDGINSFSCLCPVGFTGLFCLHEINECSSHPCLNEGTCVDGLGTYHCSCPLGYTGKNCQTLVNLCSRSPCKNKGTCIQDKAESRCRCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCVDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPNPRDCESGCASSPCQHGGSCHPQRQPPYYSCQCAPPFWGSRCELYTAPPSTPPATCLSQYCADKARDGVCDEACNSHACQWDGGDCSLTMENPWANCSSPLPCWDYINNQCDELCNTAECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSIPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGSGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKNTMPTSLPNLAKEAKDAKGSRRKKSLSEKVQLSESSVTLSPVDSLESPHTYVSDTTSSPMITSPGILQASPNPMLATAAPPASVHAQHALSFSNLHEMQPLAHGASTVLPSVSQLLSHHHIVPPSSGSAGSLSRLHPVPVPADWMNRMEVNETQYNEMFGMVLAPAEGTHPSIAPQSRPPEGKHITTPREPLPPIVTFQLIPKGSIAQPAGAPQPQSTCPPAVTGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 72 - protein, 2455 aa, Cavia porcellus
  misc_feature (1)..(2455): Guinea pig Notch2, with signal sequence (amino acids 1-9)
MYLFCFVLALQCRDDYEPCVNEGICVTYHNGTGYCKCPEGFLGEYCQHRDPCEKNRCQNGGTCVAQAMLGRATCRCALGFTGEDCQYSTSHPCFVNPPCQNGGTCHMLSWDTYECTCQVGFTGKLCQWIDACLSQPCANGSTCTTVANQFSCKCLAGFTGQKCETDVNECDIPGQCQNGGTCLNLPGSYQCQCSQGFTGQHCDNPYVPCAPSPCVNGGTCRQTGDFTFECSCLPGFEGSTCERNIDDCPNHRCQNGGVCVDGVNTYNCRCPPQWTGQFCTEDVDECLLQPNACQNGGTCTNRNGGYGCVCVNGWSGDDCSENIDDCAFASCTPGSTCIDRVASFSCMCPEGKAGLLCHLDDACISNPCHKGALCDTNPLNGHYICTCPQGYKGADCTEDVDECAMTNSNPCEHAGKCVNTDGAFHCECLKGYAGPRCEMDINECHSDPCQNDATCLDKIGGFTCLCMPGFKGVHCEIEINECQSNPCVNNGQCVDKVNRFQCLCPPGFTGPVCQIDIDDCSSTPCLNGAKCIDHPNGYECQCATGFTGLLCEENIDNCDPDPCHHGQCQDGIDSYTCICNPGYMGAICSDQIDECYSSPCLNEGRCIDLVNGYQCNCQPGTSGVNCEINFDDCASSPCVNGTCVDGISRYSCVCSPGFTGQRCNVDIDECASNPCRKGATCINDVNGFRCICPEGPHHPSCYSQVNECLSNPCIHGSCIGGLSGYKCLCDAGWVGINCEVDKNECLSNPCQNGGTCDNLVNGYKCTCKKGFKGYNCQVNIDECASNPCLNQGTCFDDVSGYTCQCALPYTGKNCQTVLAPCSPNPCENAAVCKEAPNFESFTCLCAPGWQGQRCTVDIDECVSKPCMNHGLCHNTQGSYMCECPPGFSGMDCEEDINDCLANPCQNGGSCVDGVNTFSCMCLPGFIGDKCQTDMNECLSEPCKNGGTCSDYVNSYTCKCQAGFDGVHCENNIDECTDSSCFNGGTCVDGINSFSCLCPVGFTGPFCLHEINECSSHPCLNEGTCVDGLGTYRCTCPLGYTGKNCQTLVNLCSQSPCKNKGTCIQEKAESRCLCPSGWTGAYCDVPNVSCDVAALNKGVLAKNLCKNSGACINAGNTHHCQCPLGYTGSYCEQQLDECASNPCKHGATCTDFIGGYRCECVPGYQGVNCEYEVDECQNQPCRNGGTCVDLVNHFKCSCPPGTRGLFCEENIDDCAGGPHCLNGGQCVDRIGGYSCRCLPGFAGERCEGDINECLSNPCNSEGSLDCIQLTNNYQCVCRSTFTGRHCETFVDVCPQKPCLNGGTCAVASNMPDGFICRCPPGFSGAKCQSSCGQVKCRKGEQCVHTAAGPRCFCPSPQDCESGCASSPCQHGGSCYPQRQPPYYSCHCSVPFGGNHCQFYMAPTSIPSDICASQYCADKARDGVCDEVCNSHACQWDGGDCSLTMEDPWANCSSPLPCWNYINNQCDELCNTAECLFDNFECQGNSKTCKYDKYCADHFKDNHCDQGCNSEECGWDGLDCAADQPENLAEGTLVIVVLMPPEQLLQDARSFLRALGTLLHTNLRIKLDSQGLPMVYPYYGEKSAAMKKQKLSRRSLPDEQEQEVAGSQVFLEIDNRQCVQDSEQCFKNTDAAAALLASHAIQGTLSYPLVSVVSESLSPKPTPLLYLLAVAVVFILFIILLGVIMAKRKRKHGSLWLPEGFTLRRDSSNHKRREPVGQDAVGLKNLSVQVSEANLIGSGTSEHWVDDEGPQPKKAKAEDEALLSEEEDPIDRRPWTQQHLEAADIRRTPSLALTPPQAEQEVDVLDVNVRGPDGCTPLMLASLRGGSSDMSDEDEDGEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMAKKSRRPNAKSTMPTSLPNLAKEAKDAKGSRRKKSLSEKVQLSESSVTLSPVDSLESPHTYVSDTTSSPMITSPGILQASPNPMLAAAAPQAPVHAQHALSFPNPHEMQPLAPGASTVLPSVSQLLSHHHIVPPGSSSAGNLSRLHPVTVPADWMNRMEMSDTQYNEMFGMVLTPAEGTHPGIAPQSRPPEGKHVPTPRETLPPIVTFQLIPKGSIAQPAGASQPQSTCPPAVAGPLPTMYQIPEMARLPGVAFPTAMMPQQDGQVAQTILPAYHPFPASVGKYPTPPSQHSYASSNAAERTPNHSGHLQGEHPYLTPSPDSPDQWSSSSPHSASDWSDVTTSPTPGSGGGGQRGPGTHMSEPPHSNMQVYA

SEQ ID NO: 73 - protein, 2473 aa, Mus musculus
  misc_feature (1)..(2473): Mouse Notch2, with signal sequence (amino acids 1-25)
MPALRPAALRALLWLWLCGAGPAHALQCRGGQEPCVNEGTCVTYHNGTGFCRCPEGFLGEYCQHRDPCEKNRCQNGGTCVPQGMLGKATCRCAPGFTGEDCQYSTSHPCFVSRPCQNGGTCHMLSRDTYECTCQVGFTGKQCQWTDACLSHPCENGSTCTSVASQFSCKCPAGLTGQKCEADINECDIPGRCQHGGTCLNLPGSYRCQCPQGFTGQHCDSPYVPCAPSPCVNGGTCRQTGDFTFECNCLPGFEGSTCERNIDDCPNHKCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGILCDENIDNCDPDPCHHGQCQDGIDSYTCICNPGYMGAICSDQIDECYSSPCLNDGRCIDLVNGYQCNCQPGTSGLNCEINFDDCASNPCMHGVCVDGINRYSCVCSPGFTGQRCNIDIDECASNPCRKGATCINDVNGFRCICPEGPHHPSCYSQVNECLSNPCIHGNCTGGLSGYKCLCDAGWVGVNCEVDKNECLSNPCQNGGTCNNLVNGYRCTCKKGFKGYNCQVNIDECASNPCLNQGTCFDDVSGYTCHCMLPYTGKNCQTVLAPCSPNPCENAAVCKEAPNFESFSCLCAPGWQGKRCTVDVDECISKPCMNNGVCHNTQGSYVCECPPGFSGMDCEEDINDCLANPCQNGGSCVDHVNTFSCQCHPGFIGDKCQTDMNECLSEPCKNGGTCSDYVNSYTCTCPAGFHGVHCENNIDECTESSCFNGGTCVDGINSFSCLCPVGFTGPFCLHDINECSSNPCLNAGTCVDGLGTYRCICPLGYTGKNCQTLVNLCSRSPCKNKGTCVQEKARPHCLCPPGWDGAYCDVLNVSCKAAALQKGVPVEHLCQHSGICINAGNTHHCQCPLGYTGSYCEEQLDECASNPCQHGATCNDFIGGYRCECVPGYQGVNCEYEVDECQNQPCQNGGTCIDLVNHFKCSCPPGTRGLLCEENIDECAGGPHCLNGGQCVDRIGGYTCRCLPGFAGERCEGDINECLSNPCSSEGSLDCVQLKNNYNCICRSAFTGRHCETFLDVCPQKPCLNGGTCAVASNMPDGFICRCPPGFSGARCQSSCGQVKCRRGEQCIHTDSGPRCFCLNPKDCESGCASNPCQHGGTCYPQRQPPHYSCRCPPSFGGSHCELYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSELESPRNAQLLYLLAVAVVIILFFILLGVIMAKRKRKHGFLWLPEGFTLRRDSSNHKRREPVGQDAVGLKNLSVQVSEANLIGSGTSEHWVDDEGPQPKKAKAEDEALLSEDDPIDRRPWTQQHLEAADIRHTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVLCGPNRSFLSLKHTPMGKKARRPNTKSTMPTSLPNLAKEAKDAKGSRRKKCLNEKVQLSESSVTLSPVDSLESPHTYVSDATSSPMITSPGILQASPTPLLAAAAPAAPVHTQHALSFSNLHDMQPLAPGASTVLPSVSQLLSHHHIAPPGSSSAGSLGRLHPVPVPADWMNRVEMNETQYSEMFGMVLAPAEGAHPGIAAPQSRPPEGKHMSTQREPLPPIVTFQLIPKGSIAQAAGAPQTQSSCPPAVAGPLPSMYQIPEMPRLPSVAFPPTMMPQQEGQVAQTIVPTYHPFPASVGKYPTPPSQHSYASSNAAERTPSHGGHLQGEHPYLTPSPESPDQWSSSSPHSASDWSDVTTSPTPGGGGGGQRGPGTHMSEPPHSNMQVYA

SEQ ID NO: 74 - protein, 212 aa, Artificial Sequence
  Synthetic: huNotch2-EGF6-10
AGSDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDEGNSHHHHHH

SEQ ID NO: 75 - protein, 235 aa, Artificial Sequence
  Synthetic: muNotch2-EGF6-10
AGSDSPYVPCAPSPCVNGGTCRQTGDFTFECNCLPGFEGSTCERNIDDCPNHKCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDEGNSGLNDIFEAQKIEWHENLYFQGHHHHHHHH

SEQ ID NO: 76 - protein, 314 aa, Artificial Sequence
  Synthetic: gpNotch2-EGF6-12
AGSDNPYVPCAPSPCVNGGTCRQTGDFTFECSCLPGFEGSTCERNIDDCPNHRCQNGGVCVDGVNTYNCRCPPQWTGQFCTEDVDECLLQPNACQNGGTCTNRNGGYGCVCVNGWSGDDCSENIDDCAFASCTPGSTCIDRVASFSCMCPEGKAGLLCHLDDACISNPCHKGALCDTNPLNGHYICTCPQGYKGADCTEDVDECAMTNSNPCEHAGKCVNTDGAFHCECLKGYAGPRCEMDINECHSDPCQNDATCLDKIGGFTCLCMPGFKGVHCEIEINEGNSGLNDIFEAQKIEWHENLYFQGHHHHHHHH

SEQ ID NO: 77 - protein, 309 aa, Artificial Sequence
  Synthetic: huNotch2-EGF6-12.R268K
AGSDSLYVPCAPSPCVNGGTCRQTGDFTFECNCLPGFEGSTCERNIDDCPNHKCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCEGNSGLNDIFEAQKIEWHENLYFQGHHHHHHHH

SEQ ID NO: 78 - protein, 314 aa, Artificial Sequence
  Synthetic: muNotch2-EGF6-12.K268R
AGSDSPYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEVNEGNSGLNDIFEAQKIEWHENLYFQGHHHHHHHH

SEQ ID NO: 79 - protein, 235 aa, Artificial Sequence
  Synthetic: ratNotch2-EGF6-10
AGSDSPYVPCAPSPCVNGGTCRQTGDFTSECHCLPGFEGSNCERNIDDCPNHKCQNGGVCVDGVNTYNCRCPPQWTGQFCTEDVDECLLQPNACQNGGTCTNRNGGYGCVCVNGWSGDDCSENIDDCAFASCTPGSTCIDRVASFSCLCPEGKAGLLCHLDDACISNPCHKGALCDTNPLNGQYICTCPQAYKGADCTEDVDEGNSGLNDIFEAQKIEWHENLYFQGHHHHHHHH

SEQ ID NO: 80 - protein, 37 aa, Artificial Sequence
  Synthetic: muNotch2 EGF7 (amino acids 260-296)
NIDDCPNHKCQNGGVCVDGVNTYNCRCPPQWTGQFCT

SEQ ID NO: 81 - protein, 2471 aa, Rattus sp.
  misc_feature (1)..(2471): Rat Notch2, with signal sequence (amino acids 1-25)
MPALRPAALRALLWLWLCGAGPAHALQCRGGQEPCVNEGTCVTYHNGTGYCRCPEGFLGEYCQHRDPCEKNRCQNGGTCVTQAMLGKATCRCAPGFTGEDCQYSTSHPCFVSRPCQNGGTCHMLSWDTYECTCQVGFTGKQCQWTDVCLSHPCENGSTCSSVANQFSCRCPAGITGQKCDADINECDIPGRCQHGGTCLNLPGSYRCQCPQRFTGQHCDSPYVPCAPSPCVNGGTCRQTGDFTSECHCLPGFEGSNCERNIDDCPNHKCQNGGVCVDGVNTYNCRCPPQWTGQFCTEDVDECLLQPNACQNGGTCTNRNGGYGCVCVNGWSGDDCSENIDDCAFASCTPGSTCIDRVASFSCLCPEGKAGLLCHLDDACISNPCHKGALCDTNPLNGQYICTCPQAYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGTLCDENIDNCDPDPCHHGQCQDGIDSYTCICNPGYMGAICSDQIDECYSSPCLNDGRCIDLVNGYQCNCQPGTSGLNCEINFDDCASNPCLHGACVDGINRYSCVCSPGFTGQRCNIDIDECASNPCRKDATCINDVNGFRCMCPEGPHHPSCYSQVNECLSSPCIHGNCTGGLSGYKCLCDAGWVGINCEVDKNECLSNPCQNGGTCNNLVNGYRCTCKKGFKGYNCQVNIDECASNPCLNQGTCLDDVSGYTCHCMLPYTGKNCQTVLAPCSPNPCENAAVCKEAPNFESFTCLCAPGWQGQRCTVDVDECVSKPCMNNGICHNTQGSYMCECPPGFSGMDCEEDINDCLANPCQNGGSCVDKVNTFSCLCLPGFVGDKCQTDMNECLSEPCKNGGTCSDYVNSYTCTCPAGFHGVHCENNIDECTESSCFNGGTCVDGINSFSCLCPVGFTGPFCLHDINECSSNPCLNSGTCVDGLGTYRCTCPLGYTGKNCQTLVNLCSPSPCKNKGTCAQEKARPRCLCPPGWDGAYCDVLNVSCKAAALQKGVPVEHLCQHSGICINAGNTHHCQCPLGYTGSYCEEQLDECASNPCQHGATCSDFIGGYRCECVPGYQGVNCEYEVDECQNQPCQNGGTCIDLVNHFKCSCPPGTRGLLCEENIDDCAGAPHCLNGGQCVDRIGGYSCRCLPGFAGERCEGDINECLSNPCSSEGSLDCIQLKNNYQCVCRSAFTGRHCETFLDVCPQKPCLNGGTCAVASNVPDGFICRCPPGFSGARCQSSCGQVKCRRGEQCVHTASGPHCFCPNHKDCESGCASNPCQHGGTCYPQRQPPYYSCRCSPPFWGSHCESYTAPTSTPPATCLSQYCADKARDGICDEACNSHACQWDGGDCSLTMEDPWANCTSSLRCWEYINNQCDELCNTAECLFDNFECQRNSKTCKYDKYCADHFKDNHCDKGCNNEECGWDGLDCAADQPENLAEGILVIVVLLPPEQLLQDSRSFLRALGTLLHTNLRIKQDSQGALMVYPYYGEKSAAMKKQKVARRSLPDEQEQEIIGSKVFLEIDNRQCVQDSDQCFKNTDAAAALLASHAIQGTLSYPLVSVVSESEDPRNTPLLYLLAVAVVIILFLILLGVIMAKRKRKHGFLWLPEGFTLRRDSSNHKRREPVGQDAVGLKNLSVQVSEANLIGSTTSEHWGDDEGPQPKKAKAEDDEALLSEDDPVDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVLCGPNRSFLSLKHTPMGKKARRPNTKSTMPTSLPNLAKEAKDVKGSRRKKCLNEKVQLSESSVTLSPVDSLESPHTYVSDATSSPMITSPGILQASPTPLLAAAPAAPVHAQHALSFSNLHEMQPLRPGASTVLPSVSQLLSHHHIVPPGSGSAGSLGRLHSVPVPSDWMNRVEMSETQYSEMFGMVLAPAEGTHPGMAAPQSRAPEGKPIPTQREPLPPIVTFQLIPKGSLAQAAGAPQTQSGCPPAVAGPLPSMYQIPEMARLPSVAFPPTMMPQQEGQVAQTIVPTYHPFPASVGKYPTPPSQHSYASSNAAERTPNHGGHLQGEHPYLTPSPESPDQWSSSSPHSASDWSDVTTSPTPGGGGGGQRGPGTHMSEPPHSNMQVYA

SEQ ID NO: 82 - protein, 193 aa, Artificial Sequence
  Synthetic: huNotch2-EGF4-7
GSQWTDACLSHPCANGSTCTTVANQFSCKCLTGFTGQKCETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDEGNSGLNDIFEAQKIEWHENLYFQGHHHHHHHH

SEQ ID NO: 83 - protein, 196 aa, Artificial Sequence
  Synthetic: huNotch2-EGF5-8
GSETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDGNSGLNDIFEAQKIEWHENLYFQGHHHHHHHH

SEQ ID NO: 84 - protein, 140 aa, Artificial Sequence
  Synthetic: huNotch2-EGF7-9
AHHHHHHGENLYFQGSERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDAGNS

SEQ ID NO: 85 - protein, 235 aa, Artificial Sequence
  Synthetic: huNotch1
AGSERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDEGNSGLNDIFEAQKIEWHENLYFQGHHHHHHHH

SEQ ID NO: 86 - protein, 312 aa, Artificial Sequence
  Synthetic: huNotch3
GSENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDEGNSGLNDIFEAQKIEWHENLYFQGHHHHHHHH

SEQ ID NO: 87 - protein, 23 aa, Artificial Sequence
  Synthetic: LC-FR1 of hu1B2.L1, hu1B2.L7, hu.1B2.v101, hu.1B2.v102, hu.1B2.v103, hu.1B2.v104
DIQMTQSPSSVSASVGDRVTITC

SEQ ID NO: 88 - protein, 15 aa, Artificial Sequence
  Synthetic: LC-FR2 of hu1B2.L1, hu1B2.L7, hu.1B2.v101, hu.1B2.v102, hu.1B2.v103, hu.1B2.v104
WYQQKPGKSPKLLIY

SEQ ID NO: 89 - protein, 32 aa, Artificial Sequence
  Synthetic: LC-FR3 of hu1B2.L1
GVPSRFSGSGSGTDYTLTISSLQPEDFATYFC

SEQ ID NO: 90 - protein, 32 aa, Artificial Sequence
  Synthetic: LC-FR3 of hu1B2.L7, hu.1B2.v101, hu.1B2.v102, hu.1B2.v103, hu.1B2.v104
GVPSRFSGSGSGTDYTLTISSLQPEDFATYYC

SEQ ID NO: 91 - protein, 10 aa, Artificial Sequence
  Synthetic: LC-FR4 of hu1B2.L1, hu1B2.L7, hu.1B2.v101, hu.1B2.v102, hu.1B2.v103, hu.1B2.v104
FGGGTKVEIK

SEQ ID NO: 92 - protein, 30 aa, Artificial Sequence
  Synthetic: HC-FR1 of hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104
EVQLVESGGGLVQPGGSLKLSCAVSGFTFS

SEQ ID NO: 93 - protein, 14 aa, Artificial Sequence
  Synthetic: HC-FR2 of hu.1B2.v1.DFS.H1, hu.1B2.H1.N54D.S51Q, hu.1B2.v2, hu.1B2.v4, hu.1B2.v8, hu.1B2.v9,
WIRQASGKGLEWIA

SEQ ID NO: 94 - protein, 14 aa, Artificial Sequence
  Synthetic: HC-FR2 of hu.1B2.H10, hu.1B2.DFS.H14, hu1B2.v101, hu1B2.v102, hu1B2.v103, hu1B2.v104
WVRQASGKGLEWVA

SEQ ID NO: 95 - protein, 32 aa, Artificial Sequence
  Synthetic: HC-FR3 of hu.1B2.v1.DFS.H1, hu.1B2.H1.N54D.S51Q, hu.1B2.v2, hu.1B2.v4, hu.1B2.v8, hu.1B2.v9
RFTISRDTSKSTLYLQMNSLKTEDTAVYYCSR

SEQ ID NO: 96 - protein, 32 aa, Artificial Sequence
  Synthetic: HC-FR3 of hu.1B2.DFS.H14, hu1B2.v101, hu1B2.v102, hu1B2.v103, hu1B2.v104
RFTISRDDSKSTLYLQMNSLKTEDTAVYYCSR

SEQ ID NO: 97 - protein, 11 aa, Artificial Sequence
  Synthetic: HC-FR4 of hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104
WGQGTLVTVSS

SEQ ID NO: 98 - protein, 112 aa, Artificial Sequence
  Synthetic: hu.3107.L1 VL
DVVMTQSPLSLPVTLGQPASISCRSSQSLVHSDGNTYLHWFQQRPGQSPRLLIYRISNRFSGVPDRFSGSGSGTDFTLKISRVEAEDVGVYYCLQSTHFPDTFGGGTKVEIK

SEQ ID NO: 99 - protein, 112 aa, Artificial Sequence
  Synthetic: hu.3107.L6 VL
DVVMTQSPDSLAVSLGERATINCRSSQSLVHSDGNTYLHWFQQKPGQPPKLLIYRISNRFSGVPDRFSGSGSGTDFTLTISSLQAEDVAVYYCLQSTHFPDTFGGGTKVEIK

SEQ ID NO: 100 - protein, 112 aa, Artificial Sequence
  Synthetic: hu.3107.L7 VL
DIVMTQSPDSLAVSLGERATINCRSSQSLVHSDGNTYLHWYQQKPGQPPKLLIYRISNRFSGVPDRFSGSGSGTDFTLTISSLQAEDVAVYYCLQSTHFPDTFGGGTKVEIK

SEQ ID NO: 101 - protein, 117 aa, Artificial Sequence
  Synthetic: hu.3107.V1-2.H1 VH
EVQLVQSGAEVKKPGASVKVSCKASGYTFSNYVIHWIRQAPGQGPEWTGYIIPGSGGTKFNEKFKGRATLTVDKSSTTAYMELSRLRSDDTVVYYCARDGAGSFTYWGQGTLVTVSS

SEQ ID NO: 102 - protein, 117 aa, Artificial Sequence
  Synthetic: hu.3107.H12 VH
EVQLVQSGAEVKKPGASVKVSCKASGYTFSNYVIHWVRQAPGQGPEWTGYIIPGSGGTKFNEKFKGRATSTVDTSSTTAYMELSRLRSDDTVVYYCARDGAGSFTYWGQGTLVTVSS

SEQ ID NO: 103 - protein, 117 aa, Artificial Sequence
  Synthetic: hu.3107.H13 VH
EVQLVQSGAEVKKPGASVKVSCKASGYTFSNYVIHWVRQAPGQGPEWTGYIIPGSGGTKFNEKFKGRATSTVDTSITTAYMELSRLRSDDTVVYYCARDGAGSFTYWGQGTLVTVSS

SEQ ID NO: 104 - protein, 117 aa, Artificial Sequence
  Synthetic: hu.3107.V5-51.H1 VH
EVQLVQSGAEVKKPGESLKISCKASGYTFSNYVIHWIRQMPGKGPEWTGYIIPGSGGTKFNEKFKGQATLSVDKSSTTAYLQWSSLKASDTAMYYCARDGAGSFTYWGQGTLVTVSS

SEQ ID NO: 105 - protein, 117 aa, Artificial Sequence
  Synthetic: hu.3107.H14 VH
EVQLVQSGAEVKKPGESLKISCKGSGYTFSNYVIHWVRQMPGKGPEWTGYIIPGSGGTKFNEKFKGQATISVDKSSTTAYLQWSSLKASDTAMYYCARDGAGSFTYWGQGTLVTVSS

SEQ ID NO: 106 - protein, 117 aa, Artificial Sequence
  Synthetic: hu.3107.H15 VH
EVQLVQSGAEVKKPGESLKISCKGSGYTFSNYVIHWVRQMPGKGPEWTGYIIPGSGGTKFNEKFKGQATISVDKSITTAYLQWSSLKASDTAMYYCARDGAGSFTYWGQGTLVTVSS

SEQ ID NO: 107 - protein, 32 aa, Artificial Sequence
  Synthetic: HC-FR3 of hu.1B2.H10
RFTISRDDSKSTAYLQMNSLKTEDTAVYYCSR

(19) United States
(12) Patent Application Publication
(10) Pub. No.: US 2023/0303682 A1
(43) Pub. Date: Sep. 28, 2023

(54) Anti-Notch2 Antibodies and Methods of Use

(71) Applicant: Genentech, Inc., South San Francisco, CA (US)

(72) Inventors: Cecilia Pui Chi Chiu, Redwood City, CA (US); Yan Wu, Foster City, CA (US); Adel Mahmoud ElSohly, Berkeley, CA (US); Daniel George Lafkas, San Francisco, CA (US); Jian Mehr-Dean Payandeh, Belmont, CA (US); Hoangdung Dang Ho, San Jose, CA (US); Siao-Ping Tsai, San Mateo, CA (US)

(73) Assignee: Genentech, Inc., South San Francisco, CA (US)

(21) Appl. No.: 18/004,550

(22) PCT Filed: Jul. 16, 2021
(86) PCT No.: PCT/US2021/041935

§ 371 Date: Jan. 6, 2023

Related U.S. Application Data
(60) Provisional application No. 63/053,034, filed on Jul. 17, 2020.

(51) Int. Cl. C07K 16/28; A61K 45/06; A61K 39/395; A61P 11/00

(52) U.S. Cl. CPC C07K 16/28; A61K 39/3955; A61K 45/06; A61P 11/00; C07K 2317/24; C07K 2317/34; C07K 2317/35; C07K 2317/55; C07K 2317/565; C07K 2317/76; C07K 2317/92

(57) ABSTRACT

The invention provides anti-Notch2 antibodies and methods of using the same.

### CROSS-REFERENCE TO RELATED APPLICATIONS

This application claims the benefit of priority of U.S. Provisional Application No. 63/053,034, filed Jul. 17, 2020, which is incorporated by reference herein in its entirety for any purpose.

### FIELD OF THE INVENTION

The present invention relates to anti-Notch2 antibodies and methods of using the same.

### BACKGROUND

The Notch receptor family is a class of evolutionarily conserved transmembrane receptors that transmit signals affecting development in organisms as diverse as sea urchins and humans. Notch receptors and their ligands, Delta and Serrate (known as Jagged in mammals), are transmembrane proteins with large extracellular domains that contain epidermal growth factor (EGF)-like repeats. The number of Notch paralogues differs between species. For example, there are four Notch receptors in mammals (Notch1-Notch4), two in Caenorhabditis elegans (LIN-12 and GLP-1) and one in Drosophila melanogaster (Notch). Notch receptors are proteolytically processed during transport to the cell surface by a furin-like protease at a site S1 on the N-terminal side of the transmembrane domain, producing an extracellular Notch (ECN) subunit and a Notch transmembrane subunit (NTM). These two subunits remain non-covalently associated and constitute the mature heterodimeric cell-surface receptor. Notch receptors and the Notch signaling pathway are reviewed, e.g., in Aster et al., Annu. Rev. Pathol. Mech. Dis. 3:587-613, 2008, and Bolos et al., Endocrine Reviews 28:339-363, 2007.

Notch2 ECN subunits contain 36 N-terminal EGF-like repeats followed by three tandemly repeated Lin 12/Notch Repeat (LNR) modules that precede the S1 site. Each LNR module contains three disulfide bonds and a group of conserved acidic and polar residues predicted to coordinate a calcium ion. Within the EGF repeat region lie binding sites for the activating ligands.

Binding of a Notch ligand to the ECN subunit initiates two successive proteolytic cleavages that occur through regulated intramembrane proteolysis. The first cleavage by a metalloprotease (ADAM10 or ADAM17) at site S2 renders the Notch transmembrane subunit susceptible to a second cleavage at site S3 close to the inner leaflet of the plasma membrane. Site S3 cleavage, which is catalyzed by a multiprotein complex containing presenilin and nicastrin and promoting γ-secretase activity, liberates the intracellular portion of the Notch transmembrane subunit, allowing it to translocate to the nucleus and activate transcription of target genes. (For review of the proteolytic cleavage of Notch, see, e.g., Sisodia et al., Nat. Rev. Neurosci. 3:281-290, 2002.)

Five Notch ligands of the Jagged and Delta-like classes have been identified in humans (Jagged1 (also termed Serrate1), Jagged2 (also termed Serrate2), Delta-like1 (also termed DLL1), Delta-like3 (also termed DLL3), and Delta-like4 (also termed DLL4)). Each of the ligands is a single-pass transmembrane protein with a conserved N-terminal Delta, Serrate, LAG-2 (DSL) motif essential for binding Notch. A series of EGF-like modules C-terminal to the DSL motif precede the membrane-spanning segment. Unlike the Notch receptors, the ligands have short cytoplasmic tails of 70-215 amino acids at the C-terminus. In addition, other types of ligands have been reported (e.g., DNER, NB3, and F3/Contactin). (For review of Notch ligands and ligand-mediated Notch activation, see, e.g., D′Souza et al., Oncogene 27:5148-5167, 2008.)

The Notch pathway functions during diverse developmental and physiological processes including those affecting neurogenesis in flies and vertebrates. In general, Notch signaling is involved in lateral inhibition, lineage decisions, and the establishment of boundaries between groups of cells (see, e.g., Bray, Molecular Cell Biology 7:678-679, 2006). Inhibition of Jagged-Notch signaling has been shown to induce a rapid loss of secretory club cells and an increase in ciliated cells in mammalian respiratory airway. Jagged blockade was also shown to reverse goblet cell metaplasia in a preclinical asthma model. See Lafkas et al., Nature 528: 127-131 (2015).

Muco-obstructive lung diseases are characterized by cough, sputum production, diffuse mucus obstruction, chronic inflammation, airway-wall ectasia, and frequent bacterial infections. In healthy individuals, the mucus layer in the lungs is transported rapidly from the distal airways toward the trachea. In individuals with muco-obstructive disease, epithelial defects in ion-fluid transport or mucin secretion, or both, leads to hyperconcentrated mucus and failed mucus transport, and mucus adhesion to the airway surfaces. This leads to mucus accumulation in the small airways that cannot be cleared by coughing, resulting in airway obstruction, infection, and inflammation.

There remains a need for treatments for muco-obstructive lung diseases. The invention described herein meets this need and provides other benefits.

### SUMMARY

The invention provides anti-Notch2 antibodies and methods of using the same.

In some embodiments, an isolated antibody that binds to human Notch2 is provided, wherein the antibody inhibits Jagged1-mediated signaling, but does not inhibit DLL1-mediated signaling. In some embodiments, an isolated antibody that binds to human Notch2 is provided, wherein the antibody inhibits Jagged1-mediated signaling to a greater extent than DLL1-mediated signaling. In some embodiments, the antibody is capable of achieving a maximum inhibition of Jagged1-mediated signaling of 100%, and a maximum inhibition of DLL1-mediated signaling of less than 80%, or less than 70%, or less than 60%. In some embodiments, the antibody does not inhibit binding of Jagged1 to Notch2. In some embodiments, the antibody does not inhibit binding of DLL1 to Notch2. In some embodiments, an isolated antibody is provided, wherein the antibody, when formatted as a bivalent IgG antibody comprising two heavy chains and two light chains, inhibits Jagged1-mediated signaling, but does not inhibit DLL1-mediated signaling.

In some embodiments, the antibody binds an epitope within the EGF7 repeat of Notch2. In some embodiments, the antibody binds an epitope within amino acids 260-296 of Notch2. In some embodiments, the antibody binds a discontinuous epitope within amino acids 260-296 of Notch2.

In some embodiments, an isolated antibody that binds Notch2 is provided, wherein the antibody binds an epitope within the EGF7 repeat of Notch2. In some embodiments, the antibody binds an epitope within amino acids 260-296 of Notch2. In some embodiments, the antibody binds a discontinuous epitope within amino acids 260-296 of Notch2.

In some embodiments, that antibody that binds Notch2 contacts arginine 268 (R268) of human Notch2. In some embodiments, the antibody does not bind a Notch2 comprising lysine 268 (K268). In some embodiments, the antibody binds a polypeptide comprising the amino acid sequence of SEQ ID NO: 74 and does not bind a polypeptide comprising the amino acid sequence of SEQ ID NO: 77. In some embodiments, the antibody binds to human Notch2 and cynomolgus monkey Notch2. In some embodiments, the antibody does not bind to mouse Notch2. In some embodiments, the antibody binds to guinea pig Notch2. In some embodiments, the antibody does not bind to human Notch1 or human Notch3.

In some embodiments, the antibody binds human Notch2 with an affinity (K_{D}) of less than 20 nM, less than 15 nM, less than 10 nM, or less than 5 nM, as determined by surface plasmon resonance.

In some embodiments, the antibody inhibits Jagged1-mediated signaling with an IC50 of less than 20 nM, less than 15 nM, less than 10 nM, or less than 5 nM. In some embodiments, inhibition of Jagged1-mediated signaling is determined using a high-content screening (HCS) assay.

In some embodiments, an antibody that binds to Notch2 comprises:
a) a heavy chain variable domain (VH) comprising (a) CDR-H1 comprising the amino acid sequence of SEQ ID NO: 4, (b) CDR-H2 comprising the amino acid sequence of SEQ ID NO: 6 or 7, and (c) CDR-H3 comprising the amino acid sequence of SEQ ID NO: 8, 9, 10, 11, or 12, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3;
b) a heavy chain variable domain (VH) comprising (a) CDR-H1 comprising the amino acid sequence of SEQ ID NO: 36, (b) CDR-H2 comprising the amino acid sequence of SEQ ID NO: 37, and (c) CDR-H3 comprising the amino acid sequence of SEQ ID NO: 38, and a light chain variable domain (VL) comprising (d) CDR-L1 comprising the amino acid sequence of SEQ ID NO: 33, (e) CDR-L2 comprising the amino acid sequence of SEQ ID NO: 34, and (f) CDR-L3 comprising the amino acid sequence of SEQ ID NO: 35;
c) a heavy chain variable domain (VH) comprising (a) CDR-H1 comprising the amino acid sequence of SEQ ID NO: 44, (b) CDR-H2 comprising the amino acid sequence of SEQ ID NO: 45, and (c) CDR-H3 comprising the amino acid sequence of SEQ ID NO: 46, and a light chain variable domain (VL) comprising (d) CDR-L1 comprising the amino acid sequence of SEQ ID NO: 41, (e) CDR-L2 comprising the amino acid sequence of SEQ ID NO: 42, and (f) CDR-L3 comprising the amino acid sequence of SEQ ID NO: 43;
d) a heavy chain variable domain (VH) comprising (a) CDR-H1 comprising the amino acid sequence of SEQ ID NO: 53, (b) CDR-H2 comprising the amino acid sequence of SEQ ID NO: 54, and (c) CDR-H3 comprising the amino acid sequence of SEQ ID NO: 55, and a light chain variable domain (VL) comprising (d) CDR-L1 comprising the amino acid sequence of SEQ ID NO: 49, (e) CDR-L2 comprising the amino acid sequence of SEQ ID NO: 50, and (f) CDR-L3 comprising the amino acid sequence of SEQ ID NO: 51 or 52; or
e) a heavy chain variable domain (VH) comprising (a) CDR-H1 comprising the amino acid sequence of SEQ ID NO: 62, (b) CDR-H2 comprising the amino acid sequence of SEQ ID NO: 63, and (c) CDR-H3 comprising the amino acid sequence of SEQ ID NO: 64, and a light chain variable domain (VL) comprising (d) CDR-L1 comprising the amino acid sequence of SEQ ID NO: 59, (e) CDR-L2 comprising the amino acid sequence of SEQ ID NO: 60, and (f) CDR-L3 comprising the amino acid sequence of SEQ ID NO: 61.

In some embodiments, the antibody comprises:
a) a VH sequence having at least 95% sequence identity to the amino acid sequence of SEQ ID NO: 14;
b) a VL sequence having at least 95% sequence identity to the amino acid sequence of SEQ ID NO: 13;
c) a VH sequence as defined in (a) and a VL sequence as defined in (b);
d) a VH sequence having at least 95% sequence identity to an amino acid sequence selected from SEQ ID NOs: 17-24, 26, 28, 30, and 32;
e) a VL sequence having at least 95% sequence identity to an amino acid sequence selected from SEQ ID NOs: 15, 16, 25, 27, 29, and 31;
f) a VH sequence as defined in (d) and a VL sequence as defined in (e);
g) a VH sequence having at least 95% sequence identity to the amino acid sequence of SEQ ID NO: 40;
h) a VL sequence having at least 95% sequence identity to the amino acid sequence of SEQ ID NO: 39;
i) a VH sequence as defined in (g) and a VL sequence as defined in (h);
j) a VH sequence having at least 95% sequence identity to an amino acid sequence selected from SEQ ID NOs: 102-106;
k) a VL sequence having at least 95% sequence identity to an amino acid sequence selected from SEQ ID NOs: 98-100;
l) a VH sequence as defined in (j) and a VL sequence as defined in (k);
m) a VH sequence having at least 95% sequence identity to the amino acid sequence of SEQ ID NO: 48;
n) a VL sequence having at least 95% sequence identity to the amino acid sequence of SEQ ID NO: 47;
o) a VH sequence as defined in (m) and a VL sequence as defined in (n);
p) a VH sequence having at least 95% sequence identity to the amino acid sequence of SEQ ID NO: 58;
q) a VL sequence having at least 95% sequence identity to the amino acid sequence of SEQ ID NO: 56 or 57;
r) a VH sequence as defined in (p) and a VL sequence as defined in (q);
s) a VH sequence having at least 95% sequence identity to the amino acid sequence of SEQ ID NO: 66;
t) a VL sequence having at least 95% sequence identity to the amino acid sequence of SEQ ID NO: 65; or
u) a VH sequence as defined in (s) and a VL sequence as defined in (t).

In some embodiments, the antibody comprises:
a) a VH sequence comprising the amino acid sequence of SEQ ID NO: 14;
b) a VL sequence comprising the amino acid sequence of SEQ ID NO: 13;
c) a VH sequence as defined in (a) and a VL sequence as defined in (b);
d) a VH sequence comprising an amino acid sequence selected from SEQ ID NOs: 17-24, 26, 28, 30, and 32;
e) a VL sequence comprising an amino acid sequence selected from SEQ ID NOs: 15, 16, 25, 27, 29, and 31;
f) a VH sequence as defined in (d) and a VL sequence as defined in (e);
g) a VH sequence comprising the amino acid sequence of SEQ ID NO: 40;
h) a VL sequence comprising the amino acid sequence of SEQ ID NO: 39;
i) a VH sequence as defined in (g) and a VL sequence as defined in (h);
j) a VH sequence comprising an amino acid sequence selected from SEQ ID NOs: 101-106;
k) a VL sequence comprising an amino acid sequence selected from SEQ ID NOs: 98-100;
l) a VH sequence as defined in (j) and a VL sequence as defined in (k);
m) a VH sequence comprising the amino acid sequence of SEQ ID NO: 48;
n) a VL sequence comprising the amino acid sequence of SEQ ID NO: 47;
o) a VH sequence as defined in (m) and a VL sequence as defined in (n);
p) a VH sequence comprising the amino acid sequence of SEQ ID NO: 58;
q) a VL sequence comprising the amino acid sequence of SEQ ID NO: 56 or 57;
r) a VH sequence as defined in (p) and a VL sequence as defined in (q);
s) a VH sequence comprising the amino acid sequence of SEQ ID NO: 66;
t) a VL sequence comprising the amino acid sequence of SEQ ID NO: 65; or
u) a VH sequence as defined in (s) and a VL sequence as defined in (t).

In some embodiments, the antibody comprises:
a) a VH sequence comprising the amino acid sequence of SEQ ID NO: 14;
b) a VL sequence comprising the amino acid sequence of SEQ ID NO: 13;
c) a VH sequence as defined in (a) and a VL sequence as defined in (b);
d) a VH sequence comprising an amino acid sequence selected from SEQ ID NOs: 17-24, 26, 28, 30, and 32;
e) a VL sequence comprising an amino acid sequence selected from SEQ ID NOs: 15, 16, 25, 27, 29, and 31;
f) a VH sequence as defined in (d) and a VL sequence as defined in (e);
g) a VH sequence comprising the amino acid sequence of SEQ ID NO: 40;
h) a VL sequence comprising the amino acid sequence of SEQ ID NO: 39;
i) a VH sequence as defined in (g) and a VL sequence as defined in (h);
j) a VH sequence comprising an amino acid sequence selected from SEQ ID NOs: 101-106;
k) a VL sequence comprising an amino acid sequence selected from SEQ ID NOs: 98-100;
l) a VH sequence as defined in (j) and a VL sequence as defined in (k);
m) a VH sequence comprising the amino acid sequence of SEQ ID NO: 48;
n) a VL sequence comprising the amino acid sequence of SEQ ID NO: 47;
o) a VH sequence as defined in (m) and a VL sequence as defined in (n);
p) a VH sequence comprising the amino acid sequence of SEQ ID NO: 58;
q) a VL sequence comprising the amino acid sequence of SEQ ID NO: 56 or 57;
r) a VH sequence as defined in (p) and a VL sequence as defined in (q);
s) a VH sequence comprising the amino acid sequence of SEQ ID NO: 66;
t) a VL sequence comprising the amino acid sequence of SEQ ID NO: 65; or
u) a VH sequence as defined in (s) and a VL sequence as defined in (t).

In some embodiments, the antibody comprises:
a) a VH sequence having at least 95% sequence identity to an amino acid sequence selected from SEQ ID NOs: 17-24, 26, 28, 30, and 32;
b) a VL sequence having at least 95% sequence identity to an amino acid sequence selected from SEQ ID NOs: 15, 16, 25, 27, 29, and 31; or
c) a VH sequence as defined in (a) and a VL sequence as defined in (b).

In some embodiments, the antibody comprises:
a) a VH sequence comprising an amino acid sequence selected from SEQ ID NOs: 17-24, 26, 28, 30, and 32;
b) a VL sequence comprising an amino acid sequence selected from SEQ ID NOs: 15, 16, 25, 27, 29, and 31; or
c) a VH sequence as defined in (a) and a VL sequence as defined in (b).

In some embodiments, the antibody:
a) comprises a VH sequence of SEQ ID NO: 26 and a VL sequence of SEQ ID NO: 25;
b) comprises a VH sequence of SEQ ID NO: 28 and a VL sequence of SEQ ID NO: 27;
c) comprises a VH sequence of SEQ ID NO: 30 and a VL sequence of SEQ ID NO: 29; or
d) comprises a VH sequence of SEQ ID NO: 32 and a VL sequence of SEQ ID NO: 31.

In some embodiments, the antibody that binds Notch2 is a monoclonal antibody. In some embodiments, the antibody is a humanized or chimeric antibody. In some embodiments, the antibody that binds Notch2 is an antibody fragment that binds Notch2. In some embodiments, the antibody fragment is selected from Fv, Fab, Fab′, Fab′-SH, and F(ab′)₂. In some embodiments, the antibody fragment is a Fab, Fab′, or Fab′-SH. In some embodiments, the antibody is a full-length antibody.

In some embodiments, an antibody is provided that competes for binding to human Notch2 with an antibody provided herein.

In some embodiments, an isolated nucleic acid is provided, which encodes an antibody that binds to Notch2 provided herein. In some embodiments, a host cell is provided that comprises the nucleic acid. In some embodiments, a host cell is provided that expresses an antibody provided herein. In some embodiments, a method of producing an antibody that binds to human Notch2 is provided, comprising culturing the host cell under conditions suitable for the expression of the antibody. In some embodiments, the method further comprising recovering the antibody from the host cell. In some embodiments, an antibody produced by the host cell is provided.

In some embodiments, a pharmaceutical composition comprising an antibody that binds Notch2 provided herein and a pharmaceutically acceptable carrier is provided. In some embodiments, the pharmaceutical composition further comprises an additional therapeutic agent. In some embodiments, the additional therapeutic agent is selected from hypertonic saline, mannitol, pulmozyme, N-acetyl cysteine, cysteamine, and a bronchodilator.

In some embodiments, an antibody that binds Notch2 or a pharmaceutical composition is provided herein for use as a medicament. In some embodiments, an antibody that binds Notch2 or a pharmaceutical composition is provided herein for use in treating a muco-obstructive lung disease. In some embodiments, the muco-obstructive lung disease is selected from chronic obstructive lung disease (COPD), cystic fibrosis, primary ciliary dyskinesia, non-cystic fibrosis bronchiectasis, and bronchiolitis.

In some embodiments, use of an antibody that binds Notch2 or a pharmaceutical composition in the manufacture of a medicament for treating a muco-obstructive lung disease is provided. In some embodiments, the muco-obstructive lung disease is selected from chronic obstructive lung disease (COPD), cystic fibrosis, primary ciliary dyskinesia, non-cystic fibrosis bronchiectasis, and bronchiolitis. In some embodiments, use of an antibody that binds Notch2 or a pharmaceutical composition in the manufacture of a medicament for reducing the number of secretory cells in a subject is provided. In some embodiments, the medicament converts secretory cells to ciliated cells. In some embodiments, the secretory cells are in the lungs of the subject. In some embodiments, the secretory cells are goblet cells.

In some embodiments, a method of treating subject with a muco-obstructive lung disease is provided, comprising administering to the subject an effective amount of an antibody that binds Notch2 provided herein, or a pharmaceutical composition provided herein. In some embodiments, the muco-obstructive lung disease is selected from chronic obstructive lung disease (COPD), cystic fibrosis, primary ciliary dyskinesia, non-cystic fibrosis bronchiectasis, and bronchiolitis. In some embodiments, a method of reducing the number of secretory cells in a subject is provided, comprising administering to the subject an effective amount of an antibody that binds Notch2 provided herein, or a pharmaceutical composition provided herein, to deplete secretory cells in the subject. In some embodiments, the method comprises converting secretory cells to ciliated cells. In some embodiments, the secretory cells are in the lungs of the subject. In some embodiments, the secretory cells are goblet cells. In some embodiments, the method further comprises administering an additional therapeutic agent to the subject. In some embodiments, the additional therapeutic agent is selected from hypertonic saline, mannitol, pulmozyme, N-acetyl cysteine, cysteamine, and a bronchodilator.

### BRIEF DESCRIPTION OF THE FIGURES

FIGS. 1A-1B show alignments of the light chain variable regions (1A) and heavy chain variable regions (1B) of rat anti-Notch2 antibody 1B2 and certain humanized versions thereof.

FIGS. 2A-2B show the light chain variable region (2A) and heavy chain variable region (2B) of rat anti-Notch2 antibody 3107.

FIGS. 3A-3B show alignments of the light chain variable regions (3A) and heavy chain variable regions (3B) of rabbit anti-Notch 2 antibodies 2338, 2430, 2430 with a C95dS substitution in the light chain, and 2621.

FIG. 4 shows epitope binning of rat.1B2, rat.3107, rb.2338, rb.2430, rb.2621, and anti-Notch ⅔ antibody OMP-59R5 (tarextumab, see U.S. Pat. No. 8,226,943 B2).

FIGS. 5A-5F show blocking of Jagged1-mediated Notch2 activity (5A, 5C, 5E) and preservation of DLL1-mediated Notch2 activity (5B, 5D, 5F) in a co-culture assay comprising cells that express Notch2 receptor and cells that express Jagged1 ligand (5A, 5C, 5E) or DLL1 ligand (5B, 5D, 5F). FIGS. 5A and 5B show change in percent activity of Jagged1-mediated signaling and DLL1-mediated signaling, respectively, with increasing antibody concentration for anti-Notch2 antibodies chimeric 1B2, and humanized versions hu1B2.v1.DFS, hu1B2.v101, hu1B2.v102, hu1B2.v103, and hu1B2.v104. FIGS. 5C and 5D show change in percent activity of Jagged1-mediated signaling and DLL1-mediated signaling, respectively, with increasing antibody concentration for rat anti-Notch2 antibody 3107. FIGS. 5E and 5F show change in percent activity of Jagged1-mediated signaling and DLL1-mediated signaling, respectively, with increasing antibody concentration for rabbit anti-Notch2 antibodies 2338, 2621, and 2430.

FIGS. 6A-6D show mRNA expression of (6A) Muc5b, (6B) Muc5ac, and (6C) Scgb1a1 in air-liquid interface (ALI) cultures of primary human bronchial epithelial cells contacted with anti-gD control antibody or rat/human chimeric anti-Notch2 antibody 1B2; and (6D) immunofluorescence analysis of anti-gD control antibody treated ALI culture (left) and anti-Notch2 antibody 1B2 treated ALI culture (right). Sections were stained with anti-Muc5b (green) for goblet cells, anti-acetylated a-tubulin (red) for ciliated cells, and DAPI (blue) for nuclear staining. A substantial decrease in goblet cells was observed in anti-Notch2 antibody 1B2 treated ALI culture.

FIGS. 7A-7B show alignments of the light chain variable regions (7A) and heavy chain variable regions (7B) of rat anti-Notch2 antibody 3107 and certain humanized versions thereof.

### DETAILED DESCRIPTION OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “anti-Notch2 antibody” and “an antibody that binds to Notch2” refer to an antibody that is capable of binding Notch2 with sufficient affinity such that the antibody is useful as a diagnostic and/or therapeutic agent in targeting Notch2. In some aspects, the extent of binding of an anti-Notch2 antibody to an unrelated, non-Notch2 protein is less than about 10% of the binding of the antibody to Notch2 as measured, e.g., by surface plasmon resonance (SPR). In certain aspects, an antibody that binds to Notch2 has a dissociation constant (K_{D}) of ≤ 1 µM, ≤ 100 nM, ≤ 10 nM, ≤ 1 nM, ≤ 0.1 nM, ≤ 0.01 nM, or ≤ 0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to Notch2 when the antibody has a K_{D} of 1 µM or less. In certain aspects, an anti-Notch2 antibody binds to an epitope of Notch2 that is conserved among Notch2 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “epitope” denotes the site on an antigen, either proteinaceous or non-proteinaceous, to which an anti-Notch2 antibody binds. Epitopes can be formed both from contiguous amino acid stretches (linear epitope) or comprise non-contiguous amino acids (i.e., discontinuous epitope or conformational epitope), e.g., coming in spatial proximity due to the folding of the antigen, i.e. by the tertiary folding of a proteinaceous antigen. Linear epitopes are typically still bound by an anti-Notch2 antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to Notch2 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also competitive binding can be used to easily determine whether an antibody binds to the same epitope of Notch2 as, or competes for binding with, a reference anti-Notch2 antibody. For example, an “antibody that binds to the same epitope” as a reference anti-Notch2 antibody refers to an antibody that blocks binding of the reference anti-Notch2 antibody to its antigen in a competition assay by 50% or more, and conversely, the reference antibody blocks binding of the antibody to its antigen in a competition assay by 50% or more. Also for example, to determine if an antibody binds to the same epitope as a reference anti-Notch2 antibody, the reference antibody is allowed to bind to Notch2 under saturating conditions. After removal of the excess of the reference anti-Notch2 antibody, the ability of an anti-Notch2 antibody in question to bind to Notch2 is assessed. If the anti-Notch2 antibody is able to bind to Notch2 after saturation binding of the reference anti-Notch2 antibody, it can be concluded that the anti-Notch2 antibody in question binds to a different epitope than the reference anti-Notch2 antibody. But, if the anti-Notch2 antibody in question is not able to bind to Notch2 after saturation binding of the reference anti-Notch2 antibody, then the anti-Notch2 antibody in question may bind to the same epitope as the epitope bound by the reference anti-Notch2 antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to Notch2, then it can be concluded that the anti-Notch2 antibody in question and the reference anti-Notch2 antibody compete for binding to Notch2.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and µ, respectively. The light chain of an antibody may be assigned to one of two types, called kappa (κ) and lambda (λ), based on the amino acid sequence of its constant domain.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some aspects, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. In some aspects, a heavy chain including an Fc region as specified herein, comprised in an antibody according to the invention, comprises an additional C-terminal glycine-lysine dipeptide (G446 and K447, numbering according to EU index). In some aspects, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3- CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some aspects, for the VL, the subgroup is subgroup kappa I or II as in Kabat et al., supra. In some aspects, for the VH, the subgroup is subgroup I or III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) CDRs defined through a combination of Chothia and Kabat: positions 24-34 (L1), 50-56 (L2) and 89-97 (L3) in VL domain, and 26-35 (H1), 50-65 (H2) and 95-102 (H3) in VH domain.

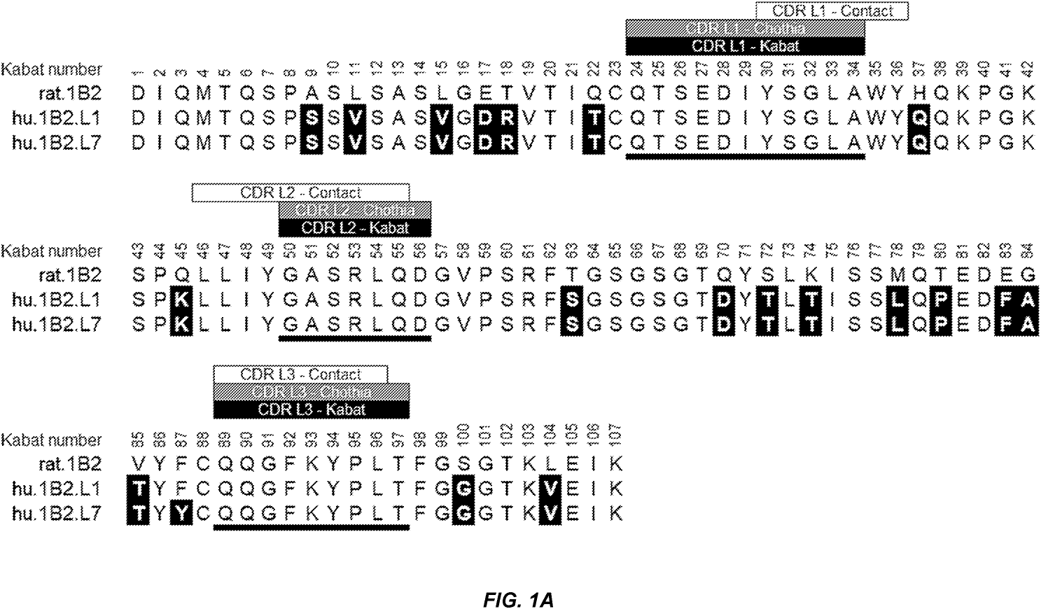

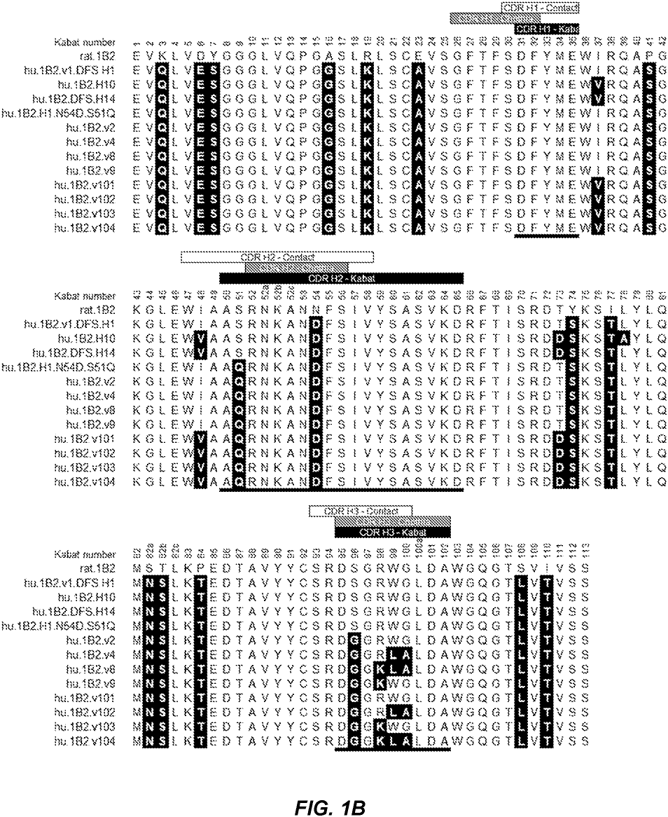

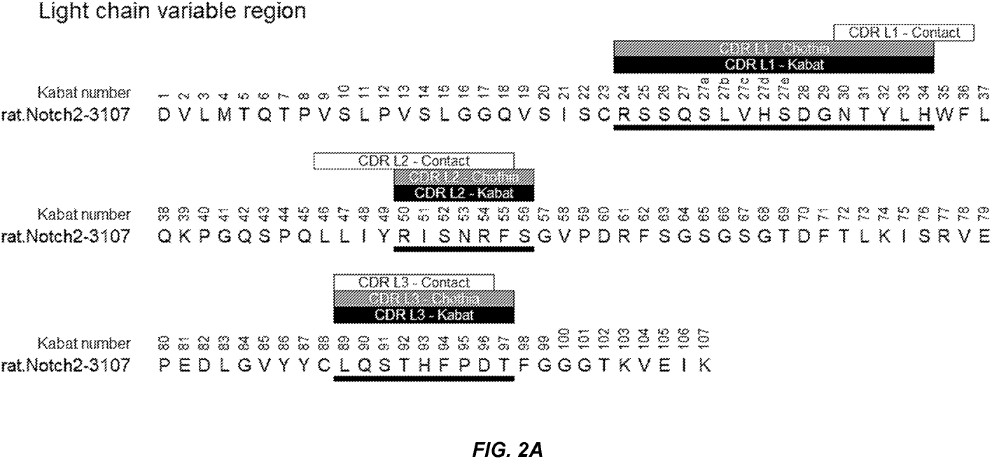

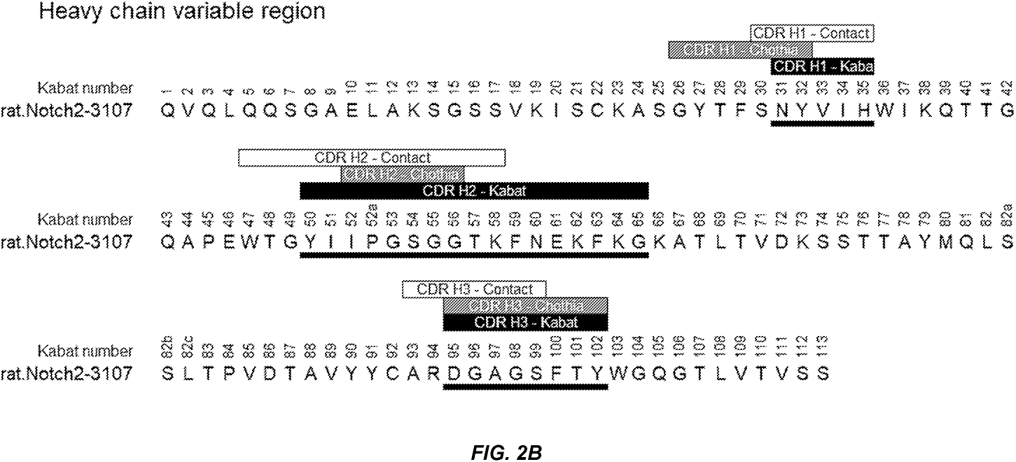

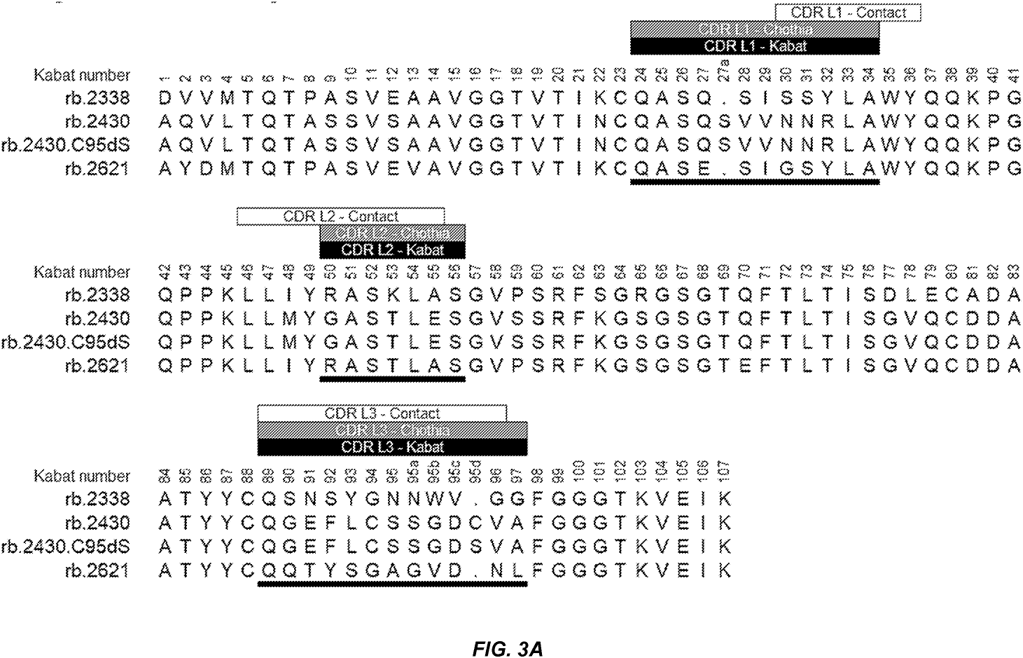

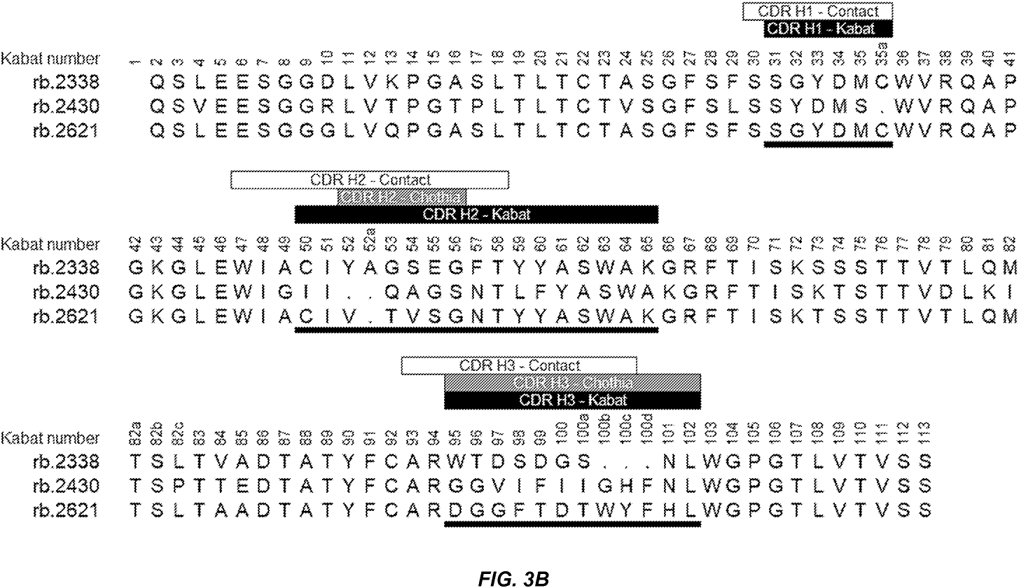

Unless otherwise indicated, the CDRs are determined according to Kabat et al., supra. One of skill in the art will understand that the CDR designations can also be determined according to Chothia, supra, McCallum, supra, or any other scientifically accepted nomenclature system. In some aspects, CDR residues comprise those identified in FIGS. 1-3 and/r the Table of Certain Sequences herein.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Notch2 antibody” refers to one or more nucleic acid molecules encoding anti-Notch2 antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “muco-obstructive lung disease” refers to a group of diseases characterized by diffuse mucus obstruction, chronic inflammation, airway-wall ectasia, and frequent bacterial infections. In muco-obstructive lung disease, hyperconcentrated mucus fails to transport effectively from the distal airways toward the trachea, and mucus adheres to airway surfaces, leading to airflow obstruction, infection, and inflammation. Nonlimiting exemplary muco-obstructive lung disease include chronic obstructive lung disease (COPD), cystic fibrosis, primary ciliary dyskinesia, non-cystic fibrosis bronchiectasis, and bronchiolitis.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Notch2”, as used herein, refers to any native Notch2 from any vertebrate source, including mammals such as primates (e.g., humans) and rodents (e.g., mice and rats), unless otherwise indicated. The term encompasses “full-length”, unprocessed Notch2 as well as any form of Notch2 that results from processing in the cell. The term also encompasses naturally occurring variants of Notch2, e.g., splice variants or allelic variants. The amino acid sequence of an exemplary human Notch2 is shown at UniProtKB/Swiss-Prot: Q04721.3 and in SEQ ID NO: 70 herein. The amino acid sequence of an exemplary cynomolgus monkey Notch2 is shown in UniProt: A0A2K5U7N0_MACFA. Another exemplary cynomolgus monkey Notch2 is shown in SEQ ID NO: 71 herein. The amino acid sequence of an exemplary guinea pig Notch2 is shown in UniProt: H0VU21 and in SEQ ID NO: 72 herein. The amino acid sequence of an exemplary guinea pig Notch2 is shown in UniProt: 035516 and in SEQ ID NO: 73 herein. The amino acid of an exemplary rat Notch2 is shown in UniProt: Q9QW30 and in SEQ ID NO: 81 herein.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXUS510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227- 258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: -10; ext: -2; Ktup = 2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In some aspects, the invention is based, in part, on antibodies that bind Notch2 and inhibit Jagged1-mediated signaling, but do not inhibit DLL1-mediated signaling. Antibodies of the invention are useful, e.g., for the diagnosis or treatment of muco-obstructive lung diseases.

### A. Exemplary Anti-Notch2 Antibodies

In some aspects, the invention provides antibodies that bind to Notch2. In some aspects, provided are isolated antibodies that bind to Notch2. In some aspects, the invention provides antibodies that specifically bind to Notch2. In certain aspects, an anti-Notch2 antibody:
Inhibits Jagged1-mediated signaling;
Does not inhibit DLL1-mediated signaling;
Does not inhibit Jagged1 binding to Notch2;
Does not inhibit DLL1 binding to Notch2;
Binds an epitope within the EGF7 repeat of Notch2;
Binds an epitope within amino acids 260-296 of Notch2;
Binds a discontinuous epitope within amino acids 260-296 of Notch2;
Contacts arginine 268 (R268) of human Notch2;
Does not bind a Notch2 comprising lysine 268 (268K);
Binds a polypeptide comprising the amino acid sequence of SEQ ID NO: 74 and does not bind a polypeptide comprising the amino acid sequence of SEQ ID NO: 77; and/or
Binds human Notch2 with an affinity (K_{D}) of less than 20 nM, less than 15 nM, less than 10 nM, or less than 5 nM, e.g., as determined by surface plasmon resonance.

### Antibodies Comprising One or More CDRS of Antibody 1b2 or Humanized Versions Thereof

In some aspects, the invention provides an anti-Notch2 antibody comprising at least one, at least two, at least three, at least four, at least five, or all six CDRs selected from (a) CDR-H1 comprising the amino acid sequence of SEQ ID NO: 4; (b) CDR-H2 comprising the amino acid sequence of SEQ ID NO: 6 or 7; (c) CDR-H3 comprising the amino acid sequence of SEQ ID NO: 8, 9, 10, 11, or 12; (d) CDR-L1 comprising the amino acid sequence of SEQ ID NO: 1; (e) CDR-L2 comprising the amino acid sequence of SEQ ID NO: 2; and (f) CDR-L3 comprising the amino acid sequence of SEQ ID NO: 3.

In some aspects, the invention provides an antibody comprising at least one, at least two, or all three VH CDR sequences selected from (a) CDR-H1 comprising the amino acid sequence of SEQ ID NO: 4; (b) CDR-H2 comprising the amino acid sequence of SEQ ID NO: 6 or 7; and (c) CDR-H3 comprising the amino acid sequence of SEQ ID NO: 8, 9, 10, 11, or 12. In some aspects, the antibody comprises CDR-H3 comprising the amino acid sequence of SEQ ID NO: 8, 9, 10, 11, or 12. In some aspects, the antibody comprises CDR-H3 comprising the amino acid sequence of SEQ ID NO: 8, 9, 10, 11, or 12, and CDR-L3 comprising the amino acid sequence of SEQ ID NO: 3. In a further aspect, the antibody comprises CDR-H3 comprising the amino acid sequence of SEQ ID NO: 8, 9, 10, 11, or 12, CDR-L3 comprising the amino acid sequence of SEQ ID NO: 3, and CDR-H2 comprising the amino acid sequence of SEQ ID NO: 2. In a further aspect, the antibody comprises (a) CDR-H1 comprising the amino acid sequence of SEQ ID NO: 4; (b) CDR-H2 comprising the amino acid sequence of SEQ ID NO: 6 or 7; and (c) CDR-H3 comprising the amino acid sequence of SEQ ID NO: 8, 9, 10, 11, or 12.

In some aspects, the invention provides an antibody comprising at least one, at least two, or all three VL CDR sequences selected from (a) CDR-L1 comprising the amino acid sequence of SEQ ID NO: 1; (b) CDR-L2 comprising the amino acid sequence of SEQ ID NO: 2; and (c) CDR-L3 comprising the amino acid sequence of SEQ ID NO: 3. In some aspects, the antibody comprises (a) CDR-L1 comprising the amino acid sequence of SEQ ID NO: 1; (b) CDR-L2 comprising the amino acid sequence of SEQ ID NO: 2; and (c) CDR-L3 comprising the amino acid sequence of SEQ ID NO: 3.

In some aspects, an antibody of the invention comprises (a) a VH domain comprising at least one, at least two, or all three VH CDR sequences selected from (i) CDR-H1 comprising the amino acid sequence of SEQ ID NO: 4, (ii) CDR-H2 comprising the amino acid sequence of SEQ ID NO: 6 or 7, and (iii) CDR-H3 comprising the amino acid sequence of SEQ ID NO: 8, 9, 10, 11, or 12; and (b) a VL domain comprising at least one, at least two, or all three VL CDR sequences selected from (i) CDR-L1 comprising the amino acid sequence of SEQ ID NO: 1, (ii) CDR-L2 comprising the amino acid sequence of SEQ ID NO: 2; and (c) CDR-L3 comprising the amino acid sequence of SEQ ID NO: 3.

In some aspects, the invention provides an antibody comprising (a) CDR-H1 comprising the amino acid sequence of SEQ ID NO: 4; (b) CDR-H2 comprising the amino acid sequence of SEQ ID NO: 6 or 7; (c) CDR-H3 comprising the amino acid sequence of SEQ ID NO: 8, 9, 10, 11, or 12; (d) CDR-L1 comprising the amino acid sequence of SEQ ID NO: 1; (e) CDR-L2 comprising the amino acid sequence of SEQ ID NO: 2; and (f) CDR-L3 comprising the amino acid sequence of SEQ ID NO: 3.

In certain aspects, any one or more amino acids of an anti-Notch2 antibody as provided herein are substituted at the following CDR positions:
in CDR-H2 (SEQ ID NO: 6): position 2
in CDR-H3 (SEQ ID NO: 8): position 2, 4, 5, and/or 6

In certain aspects, the substitutions are conservative substitutions, as provided herein. In certain aspects, any one or more of the following substitutions may be made in any combination:
in CDR-H2 (SEQ ID NO: 6): S2Q (S51Q by Kabat numbering)
in CDR-H3 (SEQ ID NO: 8): S2G (S96G by Kabat numbering); R4K (R98K by Kabat numbering); W5L (W99L by Kabat numbering); and/or G6A (G100A by Kabat numbering).

In any of the aspects provided herein, an anti-Notch2 antibody is humanized. In some aspects, an anti-Notch2 antibody further comprises an acceptor human framework, e.g. a human immunoglobulin framework or a human consensus framework. In some aspects, an anti-Notch2 antibody comprises a VH comprising an FR1 sequence of SEQ ID NO: 92; an FR2 sequence of SEQ ID NO: 93 or 94; an FR3 sequence of SEQ ID NO: 95, 96, or 107, and/or an FR4 sequence of SEQ ID NO: 97. In some aspects, an anti-Notch2 antibody comprises a VL comprising an FR1 sequence of SEQ ID NO: 87; an FR2 sequence of SEQ ID NO: 88; an FR3 sequence of SEQ ID NO: 89 or 90, and/or an FR4 sequence of SEQ ID NO: 91.

In some aspects, an anti-Notch2 antibody comprises a VH domain comprising one or more heavy chain framework sequences selected from (a) a heavy chain frame work region 1 (HC-FR1) of SEQ ID NO: 92, (b) a heavy chain frame work region 2 (HC-FR2) of SEQ ID NO: 93 or 94, (c) a heavy chain frame work region 3 (HC-FR3) of SEQ ID NO: 95, 96, or 107, and (d) a heavy chain frame work region 4 (HC-FR4) of SEQ ID NO: 97.

In some aspects, an anti-Notch2 antibody comprises a VH domain comprising aHC-FR1 of SEQ ID NO: 92. In some aspects, an anti-Notch2 antibody comprises a VH domain comprising a HC-FR2 of SEQ ID NO: 93 or 94. In some aspects, an anti-Notch2 antibody comprises a VH domain comprising a HC-FR3 of SEQ ID NO: 95, 96, or 107. In some aspects, an anti-Notch2 antibody comprises a VH domain comprising a HC-FR4 of SEQ ID NO: 97.

In some aspects, an anti-Notch2 antibody comprises a VH domain comprising aHC-FR1 of at least 85%, 86%, 87%, 88%, 89%, 90%, 91%, 92%, 93%, 94%, 95%, 96%, 97%, 98% or 99% sequence identity with SEQ ID NO: 92. In some aspects, the VH domain comprises a HC-FR1 of at least 95% sequence identity with SEQ ID NO: 92. In some aspects, the VH domain comprises a HC-FR1 of at least 98% sequence identity with SEQ ID NO: 92.

In some aspects, an anti-Notch2 antibody comprises a VH domain comprising aHC-FR2 of at least 85%, 86%, 87%, 88%, 89%, 90%, 91%, 92%, 93%, 94%, 95%, 96%, 97%, 98% or 99% sequence identity to SEQ ID NO: 93 or 94. In some aspects, the VH domain comprises a HC-FR2 of at least 95% sequence identity with SEQ ID NO: 93 or 94. In some aspects, the VH domain comprises a HC-FR2 of at least 98% sequence identity with SEQ ID NO: 93 or 94.

In some aspects, an anti-Notch2 antibody comprises a VH domain comprising aHC-FR3 of at least 85%, 86%, 87%, 88%, 89%, 90%, 91%, 92%, 93%, 94%, 95%, 96%, 97%, 98% or 99% sequence identity to SEQ ID NO: 95, 96, or 107. In some aspects, the VH domain comprises a HC-FR3 of at least 95% sequence identity with SEQ ID NO: 95, 96, or 107. In some aspects, the VH domain comprises a HC-FR3 of at least 98% sequence identity with SEQ ID NO: 95, 96, or 107.

In some aspects, an anti-Notch2 antibody comprises a VH domain comprising a HC-FR4 of at least 85%, 86%, 87%, 88%, 89%, 90%, 91%, 92%, 93%, 94%, 95%, 96%, 97%, 98% or 99% sequence identity to SEQ ID NO: 97. In some aspects, the VH domain comprises aHC-FR4 of at least 95% sequence identity with SEQ ID NO: 97. In some aspects, the VH domain comprises a HC-FR4 of at least 98% sequence identity with SEQ ID NO: 97.

In some aspects, an anti-Notch2 antibody comprises a VL domain comprising one or more light chain framework sequences selected from (a) a light chain frame work region 1 (LC-FR1) of SEQ ID NO: 87, (b) a light chain frame work region 2 (LC-FR2) of SEQ ID NO: 88, (c) a light chain frame work region 3 (LC-FR3) of SEQ ID NO: 89 or 90, and (d) a light chain frame work region 4 (LC-FR4) of SEQ ID NO: 91.

In some aspects, an anti-Notch2 antibody comprises a VL domain comprising a LC-FR1 of SEQ ID NO: 87. In some aspects, an anti-Notch2 antibody comprises a VL domain comprising a LC-FR2 of SEQ ID NO: 88. In some aspects, an anti-Notch2 antibody comprises a VL domain comprising a LC-FR3 of SEQ ID NO: 89 or 90. In some aspects, an anti-Notch2 antibody comprises a VL domain comprising a LC-FR4 of SEQ ID NO: 91.

In some aspects, an anti-Notch2 antibody comprises a VL domain comprising a LC-FR1 of at least 85%, 86%, 87%, 88%, 89%, 90%, 91%, 92%, 93%, 94%, 95%, 96%, 97%, 98% or 99% sequence identity to SEQ ID NO: 87. In some aspects, the VL domain comprises a LC-FR1 of at least 95% sequence identity with SEQ ID NO: 87. In some aspects, the VL domain comprises a LC-FR1 of at least 98% sequence identity with SEQ ID NO: 87.

In some aspects, an anti-Notch2 antibody comprises a VL domain comprising a LC-FR2 of at least 85%, 86%, 87%, 88%, 89%, 90%, 91%, 92%, 93%, 94%, 95%, 96%, 97%, 98% or 99% sequence identity to SEQ ID NO: 88. In some aspects, the VL domain comprises a LC-FR2 of at least 95% sequence identity with SEQ ID NO: 88. In some aspects, the VL domain comprises a LC-FR2 of at least 98% sequence identity with SEQ ID NO: 88.

In some aspects, an anti-Notch2 antibody comprises a VL domain comprising a LC-FR3 of at least 85%, 86%, 87%, 88%, 89%, 90%, 91%, 92%, 93%, 94%, 95%, 96%, 97%, 98% or 99% sequence identity to SEQ ID NO: 89 or 90. In some aspects, the VL domain comprises a LC-FR3 of at least 95% sequence identity with SEQ ID NO: 89 or 90. In some aspects, the VL domain comprises a LC-FR3 of at least 98% sequence identity with SEQ ID NO: 89 or 90.

In some aspects, an anti-Notch2 antibody comprises a VL domain comprising a LC-FR4 of at least 85%, 86%, 87%, 88%, 89%, 90%, 91%, 92%, 93%, 94%, 95%, 96%, 97%, 98% or 99% sequence identity to SEQ ID NO: 91. In some aspects, the VL domain comprises a LC-FR1 of at least 95% sequence identity with SEQ ID NO: 91. In some aspects, the VL domain comprises a LC-FR1 of at least 98% sequence identity with SEQ ID NO: 91.

In some aspects, an anti-Notch2 antibody comprises one or more of the CDR sequences of the VH of SEQ ID NO: 14, 17, 18, 19, 20, 21, 22, 23, 24, 26, 28, 30, or 32. In another embodiment, an anti-Notch2 antibody comprises one or more of the CDR sequences of the VL of SEQ ID NO: 13, 15, 16, 25, 27, 29, or 31. In another embodiment, an anti-Notch 2 antibody comprises the CDR sequences of the VH of SEQ ID NO: 14, 17, 18, 19, 20, 21, 22, 23, 24, 26, 28, 30, or 32, and the CDR sequences of the VL of SEQ ID NO: 13, 15, 16, 25, 27, 29, or 31.

In a further aspect, an anti-Notch2 antibody comprises the CDR-H1, CDR-H2 and CDR-H3 amino acid sequences of the VH domain of SEQ ID NO: 14, 17, 18, 19, 20, 21, 22, 23, 24, 26, 28, 30, or 32, and the CDR-L1, CDR-L2 and CDR-L3 amino acid sequences of the VL domain of SEQ ID NO: 13, 15, 16, 25, 27, 29, or 31 .

In some aspects, an anti-Notch2 antibody comprises one or more of the heavy chain CDR amino acid sequences of the VH domain of SEQ ID NO: 14, 17, 18, 19, 20, 21, 22, 23, 24, 26, 28, 30, or 32, and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 14, 17, 18, 19, 20, 21, 22, 23, 24, 26, 28, 30, or 32. In some aspects, the anti-Notch2 antibody comprises the three heavy chain CDR amino acid sequences of the VH domain of SEQ ID NO: 14, 17, 18, 19, 20, 21, 22, 23, 24, 26, 28, 30, or 32, and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 14, 17, 18, 19, 20, 21, 22, 23, 24, 26, 28, 30, or 32. In some aspects, the anti-Notch2 antibody comprises the three heavy chain CDR amino acid sequences of the VH domain of SEQ ID NO: 14, 17, 18, 19, 20, 21, 22, 23, 24, 26, 28, 30, or 32, and a framework of at least 95% sequence identity to the framework amino acid sequence of the VH domain of SEQ ID NO: 14, 17, 18, 19, 20, 21, 22, 23, 24, 26, 28, 30, or 32. In some aspects, the anti-Notch2 antibody comprises the three heavy chain CDR amino acid sequences of the VH domain of SEQ ID NO: 14, 17, 18, 19, 20, 21, 22, 23, 24, 26, 28, 30, or 32, and a framework of at least 98% sequence identity to the framework amino acid sequence of the VH domain of SEQ ID NO: 14, 17, 18, 19, 20, 21, 22, 23, 24, 26, 28, 30, or 32.

In some aspects, an anti-Notch2 antibody comprises one or more of the light chain CDR amino acid sequences of the VL domain of SEQ ID NO: 13, 15, 16, 25, 27, 29, or 31 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 13, 15, 16, 25, 27, 29, or 31. In some aspects, the anti-Notch2 antibody comprises the three light chain CDR amino acid sequences of the VL domain of SEQ ID NO: 13, 15, 16, 25, 27, 29, or 31, and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 13, 15, 16, 25, 27, 29, or 31. In some aspects, the anti-Notch2 antibody comprises the three light chain CDR amino acid sequences of the VL domain of SEQ ID NO: 13, 15, 16, 25, 27, 29, or 31, and a framework of at least 95% sequence identity to the framework amino acid sequence of the VL domain of SEQ ID NO: 13, 15, 16, 25, 27, 29, or 31. In some aspects, the anti-Notch2 antibody comprises the three light chain CDR amino acid sequences of the VL domain of SEQ ID NO: 13, 15, 16, 25, 27, 29, or 31, and a framework of at least 98% sequence identity to the framework amino acid sequence of the VL domain of SEQ ID NO: 13, 15, 16, 25, 27, 29, or 31.

In some aspects, the anti-Notch2 antibody comprises (a) CDR-H1 comprising the amino acid sequence of SEQ ID NO: 4; (b) CDR-H2 comprising the amino acid sequence of SEQ ID NO: 6 or 7; (c) CDR-H3 comprising the amino acid sequence of SEQ ID NO: 8, 9, 10, 11, or 12; (d) CDR-L1 comprising the amino acid sequence of SEQ ID NO: 1; (e) CDR-L2 comprising the amino acid sequence of SEQ ID NO: 2; and (f) CDR-L3 comprising the amino acid sequence of SEQ ID NO: 3, and a VH domain having at least 90%, 91%, 92%, 93%, 94%, 95%, 96%, 97%, 98%, 99%, or 100% sequence identity to the amino acid sequence of SEQ ID NO: 14, 17, 18, 19, 20, 21, 22, 23, 24, 26, 28, 30, or 32, and a VL domain having at least 90%, 91%, 92%, 93%, 94%, 95%, 96%, 97%, 98%, 99%, or 100% sequence identity to the amino acid sequence of SEQ ID NO: 13, 15, 16, 25, 27, 29, or 31. In some aspects, the VH domain has at least 95% sequence identity to the amino acid sequence of SEQ ID NO: 14, 17, 18, 19, 20, 21, 22, 23, 24, 26, 28, 30, or 32. In some aspects, the VL domain has at least 95% sequence identity to the amino acid sequence of SEQ ID NO: 13, 15, 16, 25, 27, 29, or 31.

In some aspects, the anti-Notch2 antibody comprises (a) CDR-H1 comprising the amino acid sequence of SEQ ID NO: 4; (b) CDR-H2 comprising the amino acid sequence of SEQ ID NO: 6 or 7; (c) CDR-H3 comprising the amino acid sequence of SEQ ID NO: 8, 9, 10, 11, or 12; (d) CDR-L1 comprising the amino acid sequence of SEQ ID NO: 1; (e) CDR-L2 comprising the amino acid sequence of SEQ ID NO: 2; and (f) CDR-L3 comprising the amino acid sequence of SEQ ID NO: 3, and a VH domain having at least 90%, 91%, 92%, 93%, 94%, 95%, 96%, 97%, 98%, 99%, or 100% sequence identity to the amino acid sequence of SEQ ID NO: 14, 17, 18, 19, 20, 21, 22, 23, 24, 26, 28, 30, or 32, and a VL domain having at least 90%, 91%, 92%, 93%, 94%, 95%, 96%, 97%, 98%, 99%, or 100% sequence identity to the amino acid sequence of SEQ ID NO: 13, 15, 16, 25, 27, 29, or 31; wherein the antibody specifically binds to Notch2. In some aspects, the VH domain has at least 95% sequence identity to the amino acid sequence of SEQ ID NO: 14, 17, 18, 19, 20, 21, 22, 23, 24, 26, 28, 30, or 32. In some aspects, the VL domain has at least 95% sequence identity to the amino acid sequence of SEQ ID NO: 13, 15, 16, 25, 27, 29, or 31. In some aspects, the antibody binds to Notch2 having a dissociation constant (K_{D}) that is up to 10 fold reduced or up to 10 fold increased when compared to the dissociation constant (K_{D}) of an antibody comprising a VH sequence of SEQ ID NO: 14, 17, 18, 19, 20, 21, 22, 23, 24, 26, 28, 30, or 32 and a VL sequence of SEQ ID NO: 13, 15, 16, 25, 27, 29, or 31.

In some aspects, an anti-Notch2 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 14, 17, 18, 19, 20, 21, 22, 23, 24, 26, 28, 30, or 32. In some aspects, an anti-Notch2 antibody comprises a heavy chain variable domain (VH) sequence having at least 95%, sequence identity to the amino acid sequence of SEQ ID NO: 14, 17, 18, 19, 20, 21, 22, 23, 24, 26, 28, 30, or 32. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 14, 17, 18, 19, 20, 21, 22, 23, 24, 26, 28, 30, or 32. In certain aspects, substitutions, insertions, or deletions occur in regions outside the CDRs (i.e., in the FRs). Optionally, the anti-Notch2 antibody comprises the VH sequence in SEQ ID NO: 14, 17, 18, 19, 20, 21, 22, 23, 24, 26, 28, 30, or 32, including post-translational modifications of that sequence. In a particular aspect, the VH comprises one, two or three CDRs selected from: (a) CDR-H1, comprising the amino acid sequence of SEQ ID NO: 4, (b) CDR-H2, comprising the amino acid sequence of SEQ ID NO: 6 or 7, and (c) CDR-H3, comprising the amino acid sequence of SEQ ID NO: 8, 9, 10, 11, or 12. In some aspects, an anti- Notch2 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 13, 15, 16, 25, 27, 29, or 31. In some aspects, an anti-Notch2 antibody comprises a light chain variable domain (VL) sequence having at least 95% sequence identity to the amino acid sequence of SEQ ID NO: 13, 15, 16, 25, 27, 29, or 31. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 13, 15, 16, 25, 27, 29, or 31. In certain aspects, the substitutions, insertions, or deletions occur in regions outside the CDRs (i.e., in the FRs). Optionally, the anti-Notch2 antibody comprises the VL sequence in SEQ ID NO: 13, 15, 16, 25, 27, 29, or 31, including post-translational modifications of that sequence. In a particular aspect, the VL comprises one, two or three CDRs selected from: (a) CDR-L1, comprising the amino acid sequence of SEQ ID NO: 1, (b) CDR-L2, comprising the amino acid sequence of SEQ ID NO: 2, and (c) CDR-L3, comprising the amino acid sequence of SEQ ID NO: 3.

In some aspects, an anti-Notch2 antibody is provided, wherein the antibody comprises a VH sequence as in any of the aspects provided above, and a VL sequence as in any of the aspects provided above. In some aspects, the antibody comprises the VH and VL sequences in SEQ ID NO: 14, 17, 18, 19, 20, 21, 22, 23, 24, 26, 28, 30, or 32, and SEQ ID NO: 13, 15, 16, 25, 27, 29, or 31, respectively, including post-translational modifications of those sequences.

### Antibodies Comprising One or More CDRs of Antibody 3107

In some aspects, the invention provides an anti-Notch2 antibody comprising at least one, at least two, at least three, at least four, at least five, or all six CDRs selected from (a) CDR-H1 comprising the amino acid sequence of SEQ ID NO: 36; (b) CDR-H2 comprising the amino acid sequence of SEQ ID NO: 37; (c) CDR-H3 comprising the amino acid sequence of SEQ ID NO: 38; (d) CDR-L1 comprising the amino acid sequence of SEQ ID NO: 33; (e) CDR-L2 comprising the amino acid sequence of SEQ ID NO: 34; and (f) CDR-L3 comprising the amino acid sequence of SEQ ID NO: 35.

In some aspects, the invention provides an antibody comprising at least one, at least two, or all three VH CDR sequences selected from (a) CDR-H1 comprising the amino acid sequence of SEQ ID NO: 36; (b) CDR-H2 comprising the amino acid sequence of SEQ ID NO: 37; and (c) CDR-H3 comprising the amino acid sequence of SEQ ID NO: 38. In some aspects, the antibody comprises CDR-H3 comprising the amino acid sequence of SEQ ID NO: 38. In some aspects, the antibody comprises CDR-H3 comprising the amino acid sequence of SEQ ID NO: 38 and CDR-L3 comprising the amino acid sequence of SEQ ID NO: 35. In a further aspect, the antibody comprises CDR-H3 comprising the amino acid sequence of SEQ ID NO: 38, CDR-L3 comprising the amino acid sequence of SEQ ID NO: 35, and CDR-H2 comprising the amino acid sequence of SEQ ID NO: 37. In a further aspect, the antibody comprises (a) CDR-H1 comprising the amino acid sequence of SEQ ID NO: 36; (b) CDR-H2 comprising the amino acid sequence of SEQ ID NO: 37; and (c) CDR-H3 comprising the amino acid sequence of SEQ ID NO: 38.

In some aspects, the invention provides an antibody comprising at least one, at least two, or all three VL CDR sequences selected from (a) CDR-L1 comprising the amino acid sequence of SEQ ID NO: 33; (b) CDR-L2 comprising the amino acid sequence of SEQ ID NO: 34; and (c) CDR-L3 comprising the amino acid sequence of SEQ ID NO: 35. In some aspects, the antibody comprises (a) CDR-L1 comprising the amino acid sequence of SEQ ID NO: 33; (b) CDR-L2 comprising the amino acid sequence of SEQ ID NO: 34; and (c) CDR-L3 comprising the amino acid sequence of SEQ ID NO: 35.

In some aspects, an antibody of the invention comprises (a) a VH domain comprising at least one, at least two, or all three VH CDR sequences selected from (i) CDR-H1 comprising the amino acid sequence of SEQ ID NO: 36, (ii) CDR-H2 comprising the amino acid sequence of SEQ ID NO: 37, and (iii) CDR-H3 comprising the amino acid sequence of SEQ ID NO: 38; and (b) a VL domain comprising at least one, at least two, or all three VL CDR sequences selected from (i) CDR-L1 comprising the amino acid sequence of SEQ ID NO: 33, (ii) CDR-L2 comprising the amino acid sequence of SEQ ID NO: 34, and (c) CDR-L3 comprising the amino acid sequence of SEQ ID NO: 35.

In some aspects, the invention provides an antibody comprising (a) CDR-H1 comprising the amino acid sequence of SEQ ID NO: 36; (b) CDR-H2 comprising the amino acid sequence of SEQ ID NO: 37; (c) CDR-H3 comprising the amino acid sequence of SEQ ID NO: 38; (d) CDR-L1 comprising the amino acid sequence of SEQ ID NO: 33; (e) CDR-L2 comprising the amino acid sequence of SEQ ID NO: 34; and (f) CDR-L3 comprising the amino acid sequence of SEQ ID NO:35.

In some aspects, an anti-Notch2 antibody comprises one or more of the CDR sequences of the VH of SEQ ID NO: 40. In another embodiment, an anti-Notch2 antibody comprises one or more of the CDR sequences of the VL of SEQ ID NO: 39. In another embodiment, an anti-Notch2 antibody comprises the CDR sequences of the VH of SEQ ID NO: 40 and the CDR sequences of the VL of SEQ ID NO: 39.

In a further aspect, an anti-Notch2 antibody comprises the CDR-H1, CDR-H2 and CDR-H3 amino acid sequences of the VH domain of SEQ ID NO: 40 and the CDR-L1, CDR-L2 and CDR-L3 amino acid sequences of the VL domain of SEQ ID NO: 39.

In some aspects, an anti-Notch2 antibody comprises one or more of the heavy chain CDR amino acid sequences of the VH domain of SEQ ID NO: 40 and a framework of at least 85%, 86%, 87%, 88%, 89%, 90%, 91%, 92%, 93%, 94%, 95%, 96%, 97%, 98%, 99% sequence identity to the framework amino acid sequence of a VH domain selected from SEQ ID NOs: 40 and 101-106. In some aspects, the anti-Notch2 antibody comprises the three heavy chain CDR amino acid sequences of the VH domain of SEQ ID NO: 40 and a framework of at least 85%, 86%, 87%, 88%, 89%, 90%, 91%, 92%, 93%, 94%, 95%, 96%, 97%, 98%, 99% sequence identity to the framework amino acid sequence of a VH domain selected from SEQ ID NOs: 40 and 101-106. In some aspects, the anti-Notch2 antibody comprises the three heavy chain CDR amino acid sequences of the VH domain of SEQ ID NO: 40 and a framework of at least 95% sequence identity to the framework amino acid sequence of a VH domain selected from SEQ ID NOs: 40 and 101-106. In some aspects, the anti-Notch2 antibody comprises the three heavy chain CDR amino acid sequences of the VH domain of SEQ ID NO: 40 and a framework of at least 98% sequence identity to the framework amino acid sequence of a VH domain selected from SEQ ID NOs: 40 and 101-106.

In some aspects, an anti-Notch2 antibody comprises one or more of the light chain CDR amino acid sequences of the VL domain of SEQ ID NO: 39 and a framework of at least 85%, 86%, 87%, 88%, 89%, 90%, 91%, 92%, 93%, 94%, 95%, 96%, 97%, 98%, 99% sequence identity to the framework amino acid sequence of a VL domain selected from SEQ ID NOs: 39 and 98-100. In some aspects, the anti-Notch2 antibody comprises the three light chain CDR amino acid sequences of the VL domain of SEQ ID NO: 39 and a framework of at least 85%, 86%, 87%, 88%, 89%, 90%, 91%, 92%, 93%, 94%, 95%, 96%, 97%, 98%, 99% sequence identity to the framework amino acid sequence of a VL domain selected from SEQ ID NOs: 39 and 98-100. In some aspects, the anti-Notch2 antibody comprises the three light chain CDR amino acid sequences of the VL domain of SEQ ID NO: 39 and a framework of at least 95% sequence identity to the framework amino acid sequence of a VL domain selected from SEQ ID NOs: 39 and 98-100. In some aspects, the anti-Notch2 antibody comprises the three light chain CDR amino acid sequences of the VL domain of SEQ ID NO: 39 and a framework of at least 98% sequence identity to the framework amino acid sequence of a VL domain selected from SEQ ID NOs: 39 and 98-100.

In some aspects, the anti-Notch2 antibody comprises (a) CDR-H1 comprising the amino acid sequence of SEQ ID NO: 36; (b) CDR-H2 comprising the amino acid sequence of SEQ ID NO: 37; (c) CDR-H3 comprising the amino acid sequence of SEQ ID NO: 38; (d) CDR-L1 comprising the amino acid sequence of SEQ ID NO: 33; (e) CDR-L2 comprising the amino acid sequence of SEQ ID NO: 34; and (f) CDR-L3 comprising the amino aci sequence of SEQ ID NO:35, and a VH domain having at least 90%, 91%, 92%, 93%, 94%, 95%, 96%, 97%, 98%, 99%, or 100% sequence identity to an amino acid sequence selected from SEQ ID NOs: 40 and 101-106, and a VL domain having at least 90%, 91%, 92%, 93%, 94%, 95%, 96%, 97%, 98%, 99%, or 100% sequence identity to an amino acid sequence selected from SEQ ID NOs: 39 and 98-100. In some aspects, the VH domain has at least 95% sequence identity to an amino acid sequence selected from SEQ ID NOs: 40 and 101-106. In some aspects, the VL domain has at least 95% sequence identity to an amino acid sequence selected from SEQ ID NOs: 39 and 98-100.

In some aspects, the anti-Notch2 antibody comprises (a) CDR-H1 comprising the amino acid sequence of SEQ ID NO: 36; (b) CDR-H2 comprising the amino acid sequence of SEQ ID NO: 37; (c) CDR-H3 comprising the amino acid sequence of SEQ ID NO: 38; (d) CDR-L1 comprising the amino acid sequence of SEQ ID NO: 33; (e) CDR-L2 comprising the amino acid sequence of SEQ ID NO: 34; and (f) CDR-L3 comprising the amino acid sequence of SEQ ID NO: 35, and a VH domain having at least 90%, 91%, 92%, 93%, 94%, 95%, 96%, 97%, 98%, 99%, or 100% sequence identity to an amino acid sequence selected from SEQ ID NOs: 40 and 101-106, and a VL domain having at least 90%, 91%, 92%, 93%, 94%, 95%, 96%, 97%, 98%, 99%, or 100% sequence identity to an amino acid sequence selected from SEQ ID NOs: 39 and 98-100; wherein the antibody specifically binds to Notch2. In some aspects, the VH domain has at least 95% sequence identity to an amino acid sequence selected from SEQ ID NOs: 40 and 101-106. In some aspects, the VL domain has at least 95% sequence identity to an amino acid sequence selected from SEQ ID NOs: 39 and 98-100. In some aspects, the antibody binds to Notch2 having a dissociation constant (K_{D}) that is up to 10 fold reduced or up to 10 fold increased when compared to the dissociation constant (K_{D}) of an antibody comprising a VH sequence of SEQ ID NO: 40 and a VL sequence of SEQ ID NO: 39.

In some aspects, an anti-Notch2 antibody comprises a heavy chain variable domain (VH) sequence having at least 90%, 91%, 92%, 93%, 94%, 95%, 96%, 97%, 98%, 99%, or 100% sequence identity to an amino acid sequence selected from SEQ ID NOs: 40 and 101-106. In some aspects, an anti-Notch2 antibody comprises a heavy chain variable domain (VH) sequence having at least 95%, sequence identity to an amino acid sequence selected from SEQ ID NOs: 40 and 101-106. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in any one of SEQ ID NOs: 40 and 101-106. In certain aspects, substitutions, insertions, or deletions occur in regions outside the CDRs (i.e., in the FRs). Optionally, the anti-Notch2 antibody comprises a VH sequence selected from SEQ ID NOs: 40 and 101-106, including post-translational modifications of that sequence. In a particular aspect, the VH comprises one, two or three CDRs selected from: (a) CDR-H1, comprising the amino acid sequence of SEQ ID NO: 36, (b) CDR-H2, comprising the amino acid sequence of SEQ ID NO: 37, and (c) CDR-H3, comprising the amino acid sequence of SEQ ID NO: 38.

In some aspects, an anti-Notch2 antibody is provided, wherein the antibody comprises a light chain variable domain (VL) sequence having at least 90%, 91%, 92%, 93%, 94%, 95%, 96%, 97%, 98%, 99%, or 100% sequence identity to an amino acid sequence selected from SEQ ID NOs: 39 and 98-100. In some aspects, an anti-Notch2 antibody comprises a light chain variable domain (VL) sequence having at least 95% sequence identity to an amino acid sequence selected from SEQ ID NOs: 39 and 98-100. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in any one of SEQ ID NOs: 39 and 98-100. In certain aspects, the substitutions, insertions, or deletions occur in regions outside the CDRs (i.e., in the FRs). Optionally, the anti-Notch2 antibody comprises a VL sequence selected from SEQ ID NOs: 39 and 98-100, including post-translational modifications of that sequence. In a particular aspect, the VL comprises one, two or three CDRs selected from: (a) CDR-L1, comprising the amino acid sequence of SEQ ID NO: 33, (b) CDR-L2, comprising the amino acid sequence of SEQ ID NO: 34, and (c) CDR-L3, comprising the amino acid sequence of SEQ ID NO: 35.

In some aspects, an anti-Notch2 antibody is provided, wherein the antibody comprises a VH sequence as in any of the aspects provided above, and a VL sequence as in any of the aspects provided above. In some aspects, the antibody comprises the VH and VL sequences in SEQ ID NO: 40, and SEQ ID NO: 39, respectively, including post-translational modifications of those sequences. In some aspects, the antibody comprises a VH sequence selected from SEQ ID NOs: 101-106 and a VL sequence selected from SEQ ID NOs: 98-100, including post-translational modifications of those sequences.

### Antibodies Comprising One or More CDRs of Antibody 2338

In some aspects, the invention provides an anti-Notch2 antibody comprising at least one, at least two, at least three, at least four, at least five, or all six CDRs selected from (a) CDR-H1 comprising the amino acid sequence of SEQ ID NO: 44; (b) CDR-H2 comprising the amino acid sequence of SEQ ID NO: 45; (c) CDR-H3 comprising the amino acid sequence of SEQ ID NO: 46; (d) CDR-L1 comprising the amino acid sequence of SEQ ID NO: 41; (e) CDR-L2 comprising the amino acid sequence of SEQ ID NO: 42; and (f) CDR-L3 comprising the amino acid sequence of SEQ ID NO: 43.

In some aspects, the invention provides an antibody comprising at least one, at least two, or all three VH CDR sequences selected from (a) CDR-H1 comprising the amino acid sequence of SEQ ID NO: 44; (b) CDR-H2 comprising the amino acid sequence of SEQ ID NO: 45; and (c) CDR-H3 comprising the amino acid sequence of SEQ ID NO: 46. In some aspects, the antibody comprises CDR-H3 comprising the amino acid sequence of SEQ ID NO: 46. In some aspects, the antibody comprises CDR-H3 comprising the amino acid sequence of SEQ ID NO: 46 and CDR-L3 comprising the amino acid sequence of SEQ ID NO: 43. In a further aspect, the antibody comprises CDR-H3 comprising the amino acid sequence of SEQ ID NO: 46, CDR-L3 comprising the amino acid sequence of SEQ ID NO: 43, and CDR-H2 comprising the amino acid sequence of SEQ ID NO: 45. In a further aspect, the antibody comprises (a) CDR-H1 comprising the amino acid sequence of SEQ ID NO: 44; (b) CDR-H2 comprising the amino acid sequence of SEQ ID NO: 45; and (c) CDR-H3 comprising the amino acid sequence of SEQ ID NO: 46.

In some aspects, the invention provides an antibody comprising at least one, at least two, or all three VL CDR sequences selected from (a) CDR-L1 comprising the amino acid sequence of SEQ ID NO: 41; (b) CDR-L2 comprising the amino acid sequence of SEQ ID NO: 42; and (c) CDR-L3 comprising the amino acid sequence of SEQ ID NO: 43. In some aspects, the antibody comprises (a) CDR-L1 comprising the amino acid sequence of SEQ ID NO: 41; (b) CDR-L2 comprising the amino acid sequence of SEQ ID NO: 42; and (c) CDR-L3 comprising the amino acid sequence of SEQ ID NO: 43.

In some aspects, an antibody of the invention comprises (a) a VH domain comprising at least one, at least two, or all three VH CDR sequences selected from (i) CDR-H1 comprising the amino acid sequence of SEQ ID NO: 44, (ii) CDR-H2 comprising the amino acid sequence of SEQ ID NO: 45, and (iii) CDR-H3 comprising the amino acid sequence of SEQ ID NO: 46; and (b) a VL domain comprising at least one, at least two, or all three VL CDR sequences selected from (i) CDR-L1 comprising the amino acid sequence of SEQ ID NO: 41, (ii) CDR-L2 comprising the amino acid sequence of SEQ ID NO: 42, and (c) CDR-L3 comprising the amino acid sequence of SEQ ID NO: 43.

In some aspects, the invention provides an antibody comprising (a) CDR-H1 comprising the amino acid sequence of SEQ ID NO: 44; (b) CDR-H2 comprising the amino acid sequence of SEQ ID NO: 45; (c) CDR-H3 comprising the amino acid sequence of SEQ ID NO: 46; (d) CDR-L1 comprising the amino acid sequence of SEQ ID NO: 41; (e) CDR-L2 comprising the amino acid sequence of SEQ ID NO: 42; and (f) CDR-L3 comprising the amino acid sequence of SEQ ID NO: 43.

In some aspects, an anti-Notch2 antibody comprises one or more of the CDR sequences of the VH of SEQ ID NO: 48. In another embodiment, an anti-Notch2 antibody comprises one or more of the CDR sequences of the VL of SEQ ID NO: 47. In another embodiment, an anti-Notch2 antibody comprises the CDR sequences of the VH of SEQ ID NO: 48 and the CDR sequences of the VL of SEQ ID NO: 47.

In a further aspect, an anti-Notch2 antibody comprises the CDR-H1, CDR-H2 and CDR-H3 amino acid sequences of the VH domain of SEQ ID NO: 48 and the CDR-L1, CDR-L2 and CDR-L3 amino acid sequences of the VL domain of SEQ ID NO: 47.

In some aspects, an anti-Notch2 antibody comprises one or more of the heavy chain CDR amino acid sequences of the VH domain of SEQ ID NO: 48 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 48. In some aspects, the anti-Notch2 antibody comprises the three heavy chain CDR amino acid sequences of the VH domain of SEQ ID NO: 48 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 48. In some aspects, the anti-Notch2 antibody comprises the three heavy chain CDR amino acid sequences of the VH domain of SEQ ID NO: 48 and a framework of at least 95% sequence identity to the framework amino acid sequence of the VH domain of SEQ ID NO: 48. In some aspects, the anti-Notch2 antibody comprises the three heavy chain CDR amino acid sequences of the VH domain of SEQ ID NO: 48 and a framework of at least 98% sequence identity to the framework amino acid sequence of the VH domain of SEQ ID NO: 48.

In some aspects, an anti-Notch2 antibody comprises one or more of the light chain CDR amino acid sequences of the VL domain of SEQ ID NO: 47 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 47. In some aspects, the anti-Notch2 antibody comprises the three light chain CDR amino acid sequences of the VL domain of SEQ ID NO: 47 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 47. In some aspects, the anti-Notch2 antibody comprises the three light chain CDR amino acid sequences of the VL domain of SEQ ID NO: 47 and a framework of at least 95% sequence identity to the framework amino acid sequence of the VL domain of SEQ ID NO: 47. In some aspects, the anti-Notch2 antibody comprises the three light chain CDR amino acid sequences of the VL domain of SEQ ID NO: 47 and a framework of at least 98% sequence identity to the framework amino acid sequence of the VL domain of SEQ ID NO: 47.

In some aspects, the anti-Notch2 antibody comprises (a) CDR-H1 comprising the amino acid sequence of SEQ ID NO: 44; (b) CDR-H2 comprising the amino acid sequence of SEQ ID NO: 45; (c) CDR-H3 comprising the amino acid sequence of SEQ ID NO: 46; (d) CDR-L1 comprising the amino acid sequence of SEQ ID NO: 41; (e) CDR-L2 comprising the amino acid sequence of SEQ ID NO: 42; and (f) CDR-L3 comprising the amino acid sequence of SEQ ID NO: 43, and a VH domain having at least 90%, 91%, 92%, 93%, 94%, 95%, 96%, 97%, 98%, 99%, or 100% sequence identity to the amino acid sequence of SEQ ID NO: 48, and a VL domain having at least 90%, 91%, 92%, 93%, 94%, 95%, 96%, 97%, 98%, 99%, or 100% sequence identity to the amino acid sequence of SEQ ID NO: 47. In some aspects, the VH domain has at least 95% sequence identity to the amino acid sequence of SEQ ID NO: 48. In some aspects, the VL domain has at least 95% sequence identity to the amino acid sequence of SEQ ID NO: 47.

In some aspects, the anti-Notch2 antibody comprises (a) CDR-H1 comprising the amino acid sequence of SEQ ID NO: 44; (b) CDR-H2 comprising the amino acid sequence of SEQ ID NO: 45; (c) CDR-H3 comprising the amino acid sequence of SEQ ID NO: 46; (d) CDR-L1 comprising the amino acid sequence of SEQ ID NO: 41; (e) CDR-L2 comprising the amino acid sequence of SEQ ID NO: 42; and (f) CDR-L3 comprising the amino acid sequence of SEQ ID NO: 43, and a VH domain having at least 90%, 91%, 92%, 93%, 94%, 95%, 96%, 97%, 98%, 99%, or 100% sequence identity to the amino acid sequence of SEQ ID NO: 48, and a VL domain having at least 90%, 91%, 92%, 93%, 94%, 95%, 96%, 97%, 98%, 99%, or 100% sequence identity to the amino acid sequence of SEQ ID NO: 47; wherein the antibody specifically binds to Notch2. In some aspects, the VH domain has at least 95% sequence identity to the amino acid sequence of SEQ ID NO: 48. In some aspects, the VL domain has at least 95% sequence identity to the amino acid sequence of SEQ ID NO: 47. In some aspects, the antibody binds to Notch2 having a dissociation constant (K_{D}) that is up to 10 fold reduced or up to 10 fold increased when compared to the dissociation constant (K_{D}) of an antibody comprising a VH sequence of SEQ ID NO: 48 and a VL sequence of SEQ ID NO: 47.

In some aspects, an anti-Notch2 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 48. In some aspects, an anti-Notch2 antibody comprises a heavy chain variable domain (VH) sequence having at least 95%, sequence identity to the amino acid sequence of SEQ ID NO: 48. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 48. In certain aspects, substitutions, insertions, or deletions occur in regions outside the CDRs (i.e., in the FRs). Optionally, the anti-Notch2 antibody comprises the VH sequence in SEQ ID NO: 48, including post-translational modifications of that sequence. In a particular aspect, the VH comprises one, two or three CDRs selected from: (a) CDR-H1, comprising the amino acid sequence of SEQ ID NO: 44, (b) CDR-H2, comprising the amino acid sequence of SEQ ID NO: 45, and (c) CDR-H3, comprising the amino acid sequence of SEQ ID NO: 46. In some aspects, an anti-Notch2 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 47. In some aspects, an anti-Notch2 antibody comprises a light chain variable domain (VL) sequence having at least 95% sequence identity to the amino acid sequence of SEQ ID NO: 47. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 47. In certain aspects, the substitutions, insertions, or deletions occur in regions outside the CDRs (i.e., in the FRs). Optionally, the anti-Notch2 antibody comprises the VL sequence in SEQ ID NO: 47, including post-translational modifications of that sequence. In a particular aspect, the VL comprises one, two or three CDRs selected from: (a) CDR-L1, comprising the amino acid sequence of SEQ ID NO: 41, (b) CDR-L2, comprising the amino acid sequence of SEQ ID NO: 42, and (c) CDR-L3, comprising the amino acid sequence of SEQ ID NO: 43.

In some aspects, an anti-Notch2 antibody is provided, wherein the antibody comprises a VH sequence as in any of the aspects provided above, and a VL sequence as in any of the aspects provided above. In some aspects, the antibody comprises the VH and VL sequences in SEQ ID NO: 48 and SEQ ID NO: 47, respectively, including post-translational modifications of those sequences.

### Antibodies Comprising One or More CDRs of Antibody 2430

In some aspects, the invention provides an anti-Notch2 antibody comprising at least one, at least two, at least three, at least four, at least five, or all six CDRs selected from (a) CDR-H1 comprising the amino acid sequence of SEQ ID NO: 53; (b) CDR-H2 comprising the amino acid sequence of SEQ ID NO: 54; (c) CDR-H3 comprising the amino acid sequence of SEQ ID NO: 55; (d) CDR-L1 comprising the amino acid sequence of SEQ ID NO: 49; (e) CDR-L2 comprising the amino acid sequence of SEQ ID NO: 50; and (f) CDR-L3 comprising the amino acid sequence of SEQ ID NO: 51 or 52.

In some aspects, the invention provides an antibody comprising at least one, at least two, or all three VH CDR sequences selected from (a) CDR-H1 comprising the amino acid sequence of SEQ ID NO: 53; (b) CDR-H2 comprising the amino acid sequence of SEQ ID NO: 54; and (c) CDR-H3 comprising the amino acid sequence of SEQ ID NO: 55. In some aspects, the antibody comprises CDR-H3 comprising the amino acid sequence of SEQ ID NO: 55. In some aspects, the antibody comprises CDR-H3 comprising the amino acid sequence of SEQ ID NO: 55 and CDR-L3 comprising the amino acid sequence of SEQ ID NO: 51 or 52. In a further aspect, the antibody comprises CDR-H3 comprising the amino acid sequence of SEQ ID NO: 55, CDR-L3 comprising the amino acid sequence of SEQ ID NO: 51 or 52, and CDR-H2 comprising the amino acid sequence of SEQ ID NO: 54. In a further aspect, the antibody comprises (a) CDR-H1 comprising the amino acid sequence of SEQ ID NO: 53; (b) CDR-H2 comprising the amino acid sequence of SEQ ID NO: 54; and (c) CDR-H3 comprising the amino acid sequence of SEQ ID NO: 55.

In some aspects, the invention provides an antibody comprising at least one, at least two, or all three VL CDR sequences selected from (a) CDR-L1 comprising the amino acid sequence of SEQ ID NO: 49; (b) CDR-L2 comprising the amino acid sequence of SEQ ID NO: 50; and (c) CDR-L3 comprising the amino acid sequence of SEQ ID NO: 51 or 52. In some aspects, the antibody comprises (a) CDR-L1 comprising the amino acid sequence of SEQ ID NO: 49; (b) CDR-L2 comprising the amino acid sequence of SEQ ID NO: 50; and (c) CDR-L3 comprising the amino acid sequence of SEQ ID NO: 51 or 52.

In some aspects, an antibody of the invention comprises (a) a VH domain comprising at least one, at least two, or all three VH CDR sequences selected from (i) CDR-H1 comprising the amino acid sequence of SEQ ID NO: 53, (ii) CDR-H2 comprising the amino acid sequence of SEQ ID NO: 54, and (iii) CDR-H3 comprising the amino acid sequence of SEQ ID NO: 55; and (b) a VL domain comprising at least one, at least two, or all three VL CDR sequences selected from (i) CDR-L1 comprising the amino acid sequence of SEQ ID NO: 49, (ii) CDR-L2 comprising the amino acid sequence of SEQ ID NO: 50, and (c) CDR-L3 comprising the amino acid sequence of SEQ ID NO: 51 or 52.

In some aspects, the invention provides an antibody comprising (a) CDR-H1 comprising the amino acid sequence of SEQ ID NO: 53; (b) CDR-H2 comprising the amino acid sequence of SEQ ID NO: 54; (c) CDR-H3 comprising the amino acid sequence of SEQ ID NO: 55; (d) CDR-L1 comprising the amino acid sequence of SEQ ID NO: 49; (e) CDR-L2 comprising the amino acid sequence of SEQ ID NO: 50; and (f) CDR-L3 comprising the amino acid sequence of SEQ ID NO: 51 or 52.

In some aspects, an anti-Notch2 antibody comprises one or more of the CDR sequences of the VH of SEQ ID NO: 58. In another embodiment, an anti-Notch2 antibody comprises one or more of the CDR sequences of the VL of SEQ ID NO: 56 or 57. In another embodiment, an anti-Notch2 antibody comprises the CDR sequences of the VH of SEQ ID NO: 58 and the CDR sequences of the VL of SEQ ID NO: 56 or 57.

In a further aspect, an anti-Notch2 antibody comprises the CDR-H1, CDR-H2 and CDR-H3 amino acid sequences of the VH domain of SEQ ID NO: 58 and the CDR-L1, CDR-L2 and CDR-L3 amino acid sequences of the VL domain of SEQ ID NO: 56 or 57.

In some aspects, an anti-Notch2 antibody comprises one or more of the heavy chain CDR amino acid sequences of the VH domain of SEQ ID NO: 58 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 58. In some aspects, the anti-Notch2 antibody comprises the three heavy chain CDR amino acid sequences of the VH domain of SEQ ID NO: 58 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 58. In some aspects, the anti-Notch2 antibody comprises the three heavy chain CDR amino acid sequences of the VH domain of SEQ ID NO: 58 and a framework of at least 95% sequence identity to the framework amino acid sequence of the VH domain of SEQ ID NO: 58. In some aspects, the anti-Notch2 antibody comprises the three heavy chain CDR amino acid sequences of the VH domain of SEQ ID NO: 58 and a framework of at least 98% sequence identity to the framework amino acid sequence of the VH domain of SEQ ID NO: 58.

In some aspects, an anti-Notch2 antibody comprises one or more of the light chain CDR amino acid sequences of the VL domain of SEQ ID NO: 56 or 57 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 56 or 57. In some aspects, the anti-Notch2 antibody comprises the three light chain CDR amino acid sequences of the VL domain of SEQ ID NO: 56 or 57 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 56 or 57. In some aspects, the anti-Notch2 antibody comprises the three light chain CDR amino acid sequences of the VL domain of SEQ ID NO: 56 or 57 and a framework of at least 95% sequence identity to the framework amino acid sequence of the VL domain of SEQ ID NO: 56 or 57. In some aspects, the anti-Notch2 antibody comprises the three light chain CDR amino acid sequences of the VL domain of SEQ ID NO: 56 or 57 and a framework of at least 98% sequence identity to the framework amino acid sequence of the VL domain of SEQ ID NO: 56 or 57.

In some aspects, the anti-Notch2 antibody comprises (a) CDR-H1 comprising the amino acid sequence of SEQ ID NO: 53; (b) CDR-H2 comprising the amino acid sequence of SEQ ID NO: 54; (c) CDR-H3 comprising the amino acid sequence of SEQ ID NO: 55; (d) CDR-L1 comprising the amino acid sequence of SEQ ID NO: 49; (e) CDR-L2 comprising the amino acid sequence of SEQ ID NO: 50; and (f) CDR-L3 comprising the amino acid sequence of SEQ ID NO: 51 or 52, and a VH domain having at least 90%, 91%, 92%, 93%, 94%, 95%, 96%, 97%, 98%, 99%, or 100% sequence identity to the amino acid sequence of SEQ ID NO: 58, and a VL domain having at least 90%, 91%, 92%, 93%, 94%, 95%, 96%, 97%, 98%, 99%, or 100% sequence identity to the amino acid sequence of SEQ ID NO: 56 or 57. In some aspects, the VH domain has at least 95% sequence identity to the amino acid sequence of SEQ ID NO: 58. In some aspects, the VL domain has at least 95% sequence identity to the amino acid sequence of SEQ ID NO: 56 or 57.

In some aspects, the anti-Notch2 antibody comprises (a) CDR-H1 comprising the amino acid sequence of SEQ ID NO: 53; (b) CDR-H2 comprising the amino acid sequence of SEQ ID NO: 54; (c) CDR-H3 comprising the amino acid sequence of SEQ ID NO: 55; (d) CDR-L1 comprising the amino acid sequence of SEQ ID NO: 49; (e) CDR-L2 comprising the amino acid sequence of SEQ ID NO: 50; and (f) CDR-L3 comprising the amino acid sequence of SEQ ID NO: 51 or 52, and a VH domain having at least 90%, 91%, 92%, 93%, 94%, 95%, 96%, 97%, 98%, 99%, or 100% sequence identity to the amino acid sequence of SEQ ID NO: 58, and a VL domain having at least 90%, 91%, 92%, 93%, 94%, 95%, 96%, 97%, 98%, 99%, or 100% sequence identity to the amino acid sequence of SEQ ID NO: 56 or 57; wherein the antibody specifically binds to Notch2. In some aspects, the VH domain has at least 95% sequence identity to the amino acid sequence of SEQ ID NO: 58. In some aspects, the VL domain has at least 95% sequence identity to the amino acid sequence of SEQ ID NO: 56 or 57. In some aspects, the antibody binds to Notch2 having a dissociation constant (K_{D}) that is up to 10 fold reduced or up to 10 fold increased when compared to the dissociation constant (K_{D}) of an antibody comprising a VH sequence of SEQ ID NO: 58 and a VL sequence of SEQ ID NO: 56 or 57.

In some aspects, an anti-Notch2 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 58. In some aspects, an anti-Notch2 antibody comprises a heavy chain variable domain (VH) sequence having at least 95%, sequence identity to the amino acid sequence of SEQ ID NO: 58. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 58. In certain aspects, substitutions, insertions, or deletions occur in regions outside the CDRs (i.e., in the FRs). Optionally, the anti-Notch2 antibody comprises the VH sequence in SEQ ID NO: 58, including post-translational modifications of that sequence. In a particular aspect, the VH comprises one, two or three CDRs selected from: (a) CDR-H1, comprising the amino acid sequence of SEQ ID NO: 53, (b) CDR-H2, comprising the amino acid sequence of SEQ ID NO: 54, and (c) CDR-H3, comprising the amino acid sequence of SEQ ID NO: 55. In some aspects, an anti-Notch2 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 56 or 57. In some aspects, an anti-Notch2 antibody comprises a light chain variable domain (VL) sequence having at least 95% sequence identity to the amino acid sequence of SEQ ID NO: 56 or 57. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 56 or 57. In certain aspects, the substitutions, insertions, or deletions occur in regions outside the CDRs (i.e., in the FRs). Optionally, the anti-Notch2 antibody comprises the VL sequence in SEQ ID NO: 56 or 57, including post-translational modifications of that sequence. In a particular aspect, the VL comprises one, two or three CDRs selected from: (a) CDR-L1, comprising the amino acid sequence of SEQ ID NO: 49, (b) CDR-L2, comprising the amino acid sequence of SEQ ID NO: 50, and (c) CDR-L3, comprising the amino acid sequence of SEQ ID NO: 51 or 52.

In some aspects, an anti-Notch2 antibody is provided, wherein the antibody comprises a VH sequence as in any of the aspects provided above, and a VL sequence as in any of the aspects provided above. In some aspects, the antibody comprises the VH and VL sequences in SEQ ID NO: 58 and SEQ ID NO: 56 or 57, respectively, including post-translational modifications of those sequences.

### Antibodies Comprising One or More CDRs of Antibody 2621

In some aspects, the invention provides an anti-Notch2 antibody comprising at least one, at least two, at least three, at least four, at least five, or all six CDRs selected from (a) CDR-H1 comprising the amino acid sequence of SEQ ID NO: 62; (b) CDR-H2 comprising the amino acid sequence of SEQ ID NO: 63; (c) CDR-H3 comprising the amino acid sequence of SEQ ID NO: 64; (d) CDR-L1 comprising the amino acid sequence of SEQ ID NO: 59; (e) CDR-L2 comprising the amino acid sequence of SEQ ID NO: 60; and (f) CDR-L3 comprising the amino acid sequence of SEQ ID NO: 61.

In some aspects, the invention provides an antibody comprising at least one, at least two, or all three VH CDR sequences selected from (a) CDR-H1 comprising the amino acid sequence of SEQ ID NO: 62; (b) CDR-H2 comprising the amino acid sequence of SEQ ID NO: 63; and (c) CDR-H3 comprising the amino acid sequence of SEQ ID NO: 64. In some aspects, the antibody comprises CDR-H3 comprising the amino acid sequence of SEQ ID NO: 59. In some aspects, the antibody comprises CDR-H3 comprising the amino acid sequence of SEQ ID NO: 60 and CDR-L3 comprising the amino acid sequence of SEQ ID NO: 61. In a further aspect, the antibody comprises CDR-H3 comprising the amino acid sequence of SEQ ID NO: 64, CDR-L3 comprising the amino acid sequence of SEQ ID NO: 61, and CDR-H2 comprising the amino acid sequence of SEQ ID NO: 63. In a further aspect, the antibody comprises (a) CDR-H1 comprising the amino acid sequence of SEQ ID NO: 62; (b) CDR-H2 comprising the amino acid sequence of SEQ ID NO: 63; and (c) CDR-H3 comprising the amino acid sequence of SEQ ID NO: 64.

In some aspects, the invention provides an antibody comprising at least one, at least two, or all three VL CDR sequences selected from (a) CDR-L1 comprising the amino acid sequence of SEQ ID NO: 59; (b) CDR-L2 comprising the amino acid sequence of SEQ ID NO: 60; and (c) CDR-L3 comprising the amino acid sequence of SEQ ID NO: 61. In some aspects, the antibody comprises (a) CDR-L1 comprising the amino acid sequence of SEQ ID NO: 59; (b) CDR-L2 comprising the amino acid sequence of SEQ ID NO: 60; and (c) CDR-L3 comprising the amino acid sequence of SEQ ID NO: 61.

In some aspects, an antibody of the invention comprises (a) a VH domain comprising at least one, at least two, or all three VH CDR sequences selected from (i) CDR-H1 comprising the amino acid sequence of SEQ ID NO: 62, (ii) CDR-H2 comprising the amino acid sequence of SEQ ID NO: 63, and (iii) CDR-H3 comprising the amino acid sequence of SEQ ID NO: 64; and (b) a VL domain comprising at least one, at least two, or all three VL CDR sequences selected from (i) CDR-L1 comprising the amino acid sequence of SEQ ID NO: 59, (ii) CDR-L2 comprising the amino acid sequence of SEQ ID NO: 60, and (c) CDR-L3 comprising the amino acid sequence of SEQ ID NO: 61.

In some aspects, the invention provides an antibody comprising (a) CDR-H1 comprising the amino acid sequence of SEQ ID NO: 62; (b) CDR-H2 comprising the amino acid sequence of SEQ ID NO: 63; (c) CDR-H3 comprising the amino acid sequence of SEQ ID NO: 64; (d) CDR-L1 comprising the amino acid sequence of SEQ ID NO: 59; (e) CDR-L2 comprising the amino acid sequence of SEQ ID NO: 60; and (f) CDR-L3 comprising the amino acid sequence of SEQ ID NO: 61.

In some aspects, an anti-Notch2 antibody comprises one or more of the CDR sequences of the VH of SEQ ID NO: 66. In another embodiment, an anti-Notch2 antibody comprises one or more of the CDR sequences of the VL of SEQ ID NO: 65. In another embodiment, an anti-Notch2 antibody comprises the CDR sequences of the VH of SEQ ID NO: 66 and the CDR sequences of the VL of SEQ ID NO: 65.

In a further aspect, an anti-Notch2 antibody comprises the CDR-H1, CDR-H2 and CDR-H3 amino acid sequences of the VH domain of SEQ ID NO: 66 and the CDR-L1, CDR-L2 and CDR-L3 amino acid sequences of the VL domain of SEQ ID NO: 65.

In some aspects, an anti-Notch2 antibody comprises one or more of the heavy chain CDR amino acid sequences of the VH domain of SEQ ID NO: 66 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 66. In some aspects, the anti-Notch2 antibody comprises the three heavy chain CDR amino acid sequences of the VH domain of SEQ ID NO: 66 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 66. In some aspects, the anti-Notch2 antibody comprises the three heavy chain CDR amino acid sequences of the VH domain of SEQ ID NO: 66 and a framework of at least 95% sequence identity to the framework amino acid sequence of the VH domain of SEQ ID NO: 66. In some aspects, the anti-Notch2 antibody comprises the three heavy chain CDR amino acid sequences of the VH domain of SEQ ID NO: 66 and a framework of at least 98% sequence identity to the framework amino acid sequence of the VH domain of SEQ ID NO: 66.

In some aspects, an anti-Notch2 antibody comprises one or more of the light chain CDR amino acid sequences of the VL domain of SEQ ID NO: 65 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 65. In some aspects, the anti-Notch2 antibody comprises the three light chain CDR amino acid sequences of the VL domain of SEQ ID NO: 65 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 65. In some aspects, the anti-Notch2 antibody comprises the three light chain CDR amino acid sequences of the VL domain of SEQ ID NO: 65 and a framework of at least 95% sequence identity to the framework amino acid sequence of the VL domain of SEQ ID NO: 65. In some aspects, the anti-Notch2 antibody comprises the three light chain CDR amino acid sequences of the VL domain of SEQ ID NO: 65 and a framework of at least 98% sequence identity to the framework amino acid sequence of the VL domain of SEQ ID NO: 65.

In some aspects, the anti-Notch2 antibody comprises (a) CDR-H1 comprising the amino acid sequence of SEQ ID NO: 62; (b) CDR-H2 comprising the amino acid sequence of SEQ ID NO: 63; (c) CDR-H3 comprising the amino acid sequence of SEQ ID NO: 64; (d) CDR-L1 comprising the amino acid sequence of SEQ ID NO: 59; (e) CDR-L2 comprising the amino acid sequence of SEQ ID NO: 60; and (f) CDR-L3 comprising the amino acid sequence of SEQ ID NO: 61, and a VH domain having at least 90%, 91%, 92%, 93%, 94%, 95%, 96%, 97%, 98%, 99%, or 100% sequence identity to the amino acid sequence of SEQ ID NO: 66, and a VL domain having at least 90%, 91%, 92%, 93%, 94%, 95%, 96%, 97%, 98%, 99%, or 100% sequence identity to the amino acid sequence of SEQ ID NO: 65. In some aspects, the VH domain has at least 95% sequence identity to the amino acid sequence of SEQ ID NO: 66. In some aspects, the VL domain has at least 95% sequence identity to the amino acid sequence of SEQ ID NO: 65.

In some aspects, the anti-Notch2 antibody comprises (a) CDR-H1 comprising the amino acid sequence of SEQ ID NO: 62; (b) CDR-H2 comprising the amino acid sequence of SEQ ID NO: 63; (c) CDR-H3 comprising the amino acid sequence of SEQ ID NO: 64; (d) CDR-L1 comprising the amino acid sequence of SEQ ID NO: 59; (e) CDR-L2 comprising the amino acid sequence of SEQ ID NO: 60; and (f) CDR-L3 comprising the amino acid sequence of SEQ ID NO: 61, and a VH domain having at least 90%, 91%, 92%, 93%, 94%, 95%, 96%, 97%, 98%, 99%, or 100% sequence identity to the amino acid sequence of SEQ ID NO: 66, and a VL domain having at least 90%, 91%, 92%, 93%, 94%, 95%, 96%, 97%, 98%, 99%, or 100% sequence identity to the amino acid sequence of SEQ ID NO: 65; wherein the antibody specifically binds to Notch2. In some aspects, the VH domain has at least 95% sequence identity to the amino acid sequence of SEQ ID NO: 66. In some aspects, the VL domain has at least 95% sequence identity to the amino acid sequence of SEQ ID NO: 65. In some aspects, the antibody binds to Notch2 having a dissociation constant (K_{D}) that is up to 10 fold reduced or up to 10 fold increased when compared to the dissociation constant (K_{D}) of an antibody comprising a VH sequence of SEQ ID NO: 66 and a VL sequence of SEQ ID NO: 65.

In some aspects, an anti-Notch2 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 66. In some aspects, an anti-Notch2 antibody comprises a heavy chain variable domain (VH) sequence having at least 95%, sequence identity to the amino acid sequence of SEQ ID NO: 66. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 66. In certain aspects, substitutions, insertions, or deletions occur in regions outside the CDRs (i.e., in the FRs). Optionally, the anti-Notch2 antibody comprises the VH sequence in SEQ ID NO: 66, including post-translational modifications of that sequence. In a particular aspect, the VH comprises one, two or three CDRs selected from: (a) CDR-H1, comprising the amino acid sequence of SEQ ID NO: 62, (b) CDR-H2, comprising the amino acid sequence of SEQ ID NO: 63, and (c) CDR-H3, comprising the amino acid sequence of SEQ ID NO: 64. In some aspects, an anti-Notch2 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 65. In some aspects, an anti-Notch2 antibody comprises a light chain variable domain (VL) sequence having at least 95% sequence identity to the amino acid sequence of SEQ ID NO: 65. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Notch2 antibody comprising that sequence retains the ability to bind to Notch2. In certain aspects, a total of 1 to 10 amino acids have been substituted, inserted and/or deleted in SEQ ID NO: 65. In certain aspects, the substitutions, insertions, or deletions occur in regions outside the CDRs (i.e., in the FRs). Optionally, the anti-Notch2 antibody comprises the VL sequence in SEQ ID NO: 65, including post-translational modifications of that sequence. In a particular aspect, the VL comprises one, two or three CDRs selected from: (a) CDR-L1, comprising the amino acid sequence of SEQ ID NO: 59, (b) CDR-L2, comprising the amino acid sequence of SEQ ID NO: 60, and (c) CDR-L3, comprising the amino acid sequence of SEQ ID NO: 61.

In some aspects, an anti-Notch2 antibody is provided, wherein the antibody comprises a VH sequence as in any of the aspects provided above, and a VL sequence as in any of the aspects provided above. In some aspects, the antibody comprises the VH and VL sequences in SEQ ID NO: 66 and SEQ ID NO: 65, respectively, including post-translational modifications of those sequences.

In a further aspect, the invention provides an antibody that binds to the same epitope as an anti-Notch2 antibody provided herein. For example, in certain aspects, an antibody is provided that binds to the same epitope as an anti-Notch2 antibody comprising a VH sequence of SEQ ID NO: 32 and a VL sequence of SEQ ID NO: 31. In certain aspects, an anti-Notch2 antibody is provided that binds to an epitope within the EGF7 repeat of Notch2. In some embodiments, an anti-Notch2 antibody is provided that binds to an epitope within amino acids 260-296 of Notch 2 (SEQ ID NO: 70). In some embodiments, an anti-Notch2 antibody is provided that binds to an epitope within amino acids 260-296 of Notch 2 (SEQ ID NO: 70).

In a further aspect, the invention provides an antibody that competes for binding to Notch2 with an anti-Notch2 antibody provided herein. For example, in certain aspects, an antibody is provided that competes for binding to Notch2 with an anti-Notch2 antibody comprising a VH sequence of SEQ ID NO: 32 and a VL sequence of SEQ ID NO: 31.

In a further aspect of the invention, an anti-Notch2 antibody according to any of the above aspects is a monoclonal antibody, including a chimeric, humanized, or human antibody. In some aspects, an anti-Notch2 antibody is an antibody fragment, e.g., a Fv, Fab, Fab′, scFv, diabody, or F(ab′)₂ fragment. In some aspects, the antibody is a full length antibody, e.g., an intact IgG1, IgG2, IgG3, or IgG4 antibody or other antibody class or isotype as defined herein.

In a further aspect, an anti-Notch2 antibody according to any of the above aspects may incorporate any of the features, singly or in combination, as described in Sections 1-8 below:

### 1. Antibody Affinity

In certain aspects, an antibody provided herein has a dissociation constant (K_{D}) of ≤ 1 µM, ≤ 100 nM, ≤ 10 nM, ≤ 1 nM, ≤ 0.1 nM, ≤ 0.01 nM, or ≤ 0.001 nM (e.g., 10⁻⁸ M or less, e.g., from 10⁻⁸ M to 10⁻¹³ M, e.g., from 10⁻⁹ M to 10⁻¹³ M).

In some aspects, K_{D} is measured using a BIACORE® surface plasmon resonance assay. For example, an assay using a BIACORE®-2000 or a BIACORE ®-3000 (BIAcore, Inc., Piscataway, NJ) is performed at 25° C. with immobilized antigen CM5 chips at ~10 response units (RU). In some aspects, carboxymethylated dextran biosensor chips (CM5, BIACORE, Inc.) are activated with N-ethyl-N′- (3-dimethylaminopropyl)-carbodiimide hydrochloride (EDC) and N-hydroxysuccinimide (NHS) according to the supplier’s instructions. Antigen is diluted with 10 mM sodium acetate, pH 4.8, to 5 µg/ml (~0.2 µM) before injection at a flow rate of 5 µl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 µl/min. Association rates (kₒₙ) and dissociation rates (k_{off}) are calculated using a simple one-to-one (1:1) Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999).

In another exemplary assay using a BIAcore™ T200 machine, for example, antibodies with human IgG1 constant regions are captured on a protein A chip to achieve approximately 300 RU. In some such embodiments, serial dilutions of purified antigen are injected in HBS-P buffer with additional 3 mM CaCl₂ at 37° C. with a flow rate of 100 µL/min. Association rates (ka) and dissociation rates (kd) are calculated using a 1:1 Langmuir binding model (BIAcore™ T200 Evaluation Software version 2.0, for example). The equilibrium dissociation constant (K_{D}) may be calculated as the ratio kd/ka.

If the on-rate exceeds 10⁶ M⁻¹ s⁻¹ by a surface plasmon resonance assay above, then the on-rate can be determined by using a fluorescent quenching technique that measures the increase or decrease in fluorescence emission intensity (excitation = 295 nm; emission = 340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In an alternative method, K_{D} is measured by a radiolabeled antigen binding assay (RIA). In some aspects, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 µg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 µl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

### 2. Antibody Fragments

In certain aspects, an antibody provided herein is an antibody fragment.

In some aspects, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In some aspects, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In some aspects, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In some aspects, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat′l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall’Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal’s chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing H_{U}M_{AB}® technology; U.S. Pat. No. 7,041,870 describing K-M MOUSE® technology, and U.S. Pat. Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 5. Library-Derived Antibodies

In certain aspects, an antibody provided herein is derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O′Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as U.S. Pat. Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 6. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for Notch2 and the other specificity is for any other antigen. In certain aspects, bispecific antibodies may bind to two (or more) different epitopes of Notch2. Multispecific (e.g., bispecific) antibodies may also be used to localize cytotoxic agents or cells to cells which express Notch2. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to Notch2 as well as another different antigen, or two different epitopes of Notch2 (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In some aspects, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for Notch2 and the other is for CD3.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 7. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### A) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| | | |
|---|---|---|
| Original Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O′Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### B) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In some aspects, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In some aspects, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ± 3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### C) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express FcγRIII only, whereas monocytes express FcyRI, FcyRII and FcyRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat′l Acad. Sci. USA 83:7059-7063 (1986)) and Hellstrom, I et al., Proc. Nat’lAcad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat′l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M.S. et al., Blood 101:1045-1052 (2003); and Cragg, M.S. and M.J. Glennie, Blood 103:2738-2743 (2004)). FcRn binding and in vivo clearance/half life determinations can also be performed using methods known in the art (see, e.g., Petkova, S.B. et al., Int′l. Immunol. 18(12):1759-1769 (2006); WO 2013/120929 Al).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcyR binding, e.g., substitutions at positions 234 and 235 of the Fc region (EU numbering of residues). In some aspects, the substitutions are L234A and L235A (LALA). In certain aspects, the antibody variant further comprises D265A and/or P329G in an Fc region derived from a human IgG₁ Fc region. In some aspects, the substitutions are L234A, L235A and P329G (LALA-PG) in an Fc region derived from a human IgG₁ Fc region. (See, e.g., WO 2012/130831). In some aspects, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall’Acqua, W.F., et al. J. Biol. Chem. 281 (2006) 23514-23524).

Fc region residues critical to the mouse Fc-mouse FcRn interaction have been identified by site-directed mutagenesis (see e.g. Dall’Acqua, W.F., et al. J. Immunol 169 (2002) 5171-5180). Residues I253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J.K., et al., Eur. J. Immunol. 24 (1994) 542). Residues I253, H310, and H435 were found to be critical for the interaction of human Fc with murine FcRn (Kim, J.K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R.L., et al., J. Biol. Chem. 276 (2001) 6591-6604). In Yeung, Y.A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In some aspects, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In some aspects, the substitutions are H310A, H433A and Y436A in an Fc region derived from a human IgG1 Fc-region. (See, e.g., WO 2014/177460 Al).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In some aspects, the substitutions are M252Y, S254T and T256E in an Fc region derived from a human IgG₁ Fc-region. See also Duncan & Winter, Nature 322:738-40 (1988); U.S. Pat. No. 5,648,260; U.S. Pat. No. 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In some aspects of all aspects as reported herein, an antibody comprising a heavy chain including a C-terminal CH3 domain as specified herein, comprises the C-terminal glycine-lysine dipeptide (G446 and K447, EU index numbering of amino acid positions). In some aspects of all aspects as reported herein, an antibody comprising a heavy chain including a C-terminal CH3 domain, as specified herein, comprises a C-terminal glycine residue (G446, EU index numbering of amino acid positions).

### D) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. No. 7,521,541, 8,30,930, 7,855,275, 9,000,130, or WO 2016040856.

### 8. Immunoconjugates

The invention also provides immunoconjugates comprising an anti-Notch2 antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In some aspects, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In some aspects, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In some aspects, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y^{90,} Re^{186,} Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### B. Recombinant Methods and Compositions

Antibodies may be produced using recombinant methods and compositions, e.g., as described in US 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In some aspects, isolated nucleic acids encoding an antibody as used in the methods as reported herein are provided.

In some aspects, a method of making an anti-Notch2 antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an anti-Notch2 antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., US 5,648,237, US 5,789,199, and US 5,840,523. (See also Charlton, K.A., In: Methods in Molecular Biology, Vol. 248, Lo, B.K.C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T.U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., US 5,959,177, US 6,040,498, US 6,420,548, US 7,125,978, and US 6,417,429 (describing PLANTIBODIESTM technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F.L. et al., J. Gen Virol. 36 (1977) 59-74); baby hamster kidney cells (BHK); mouse sertoli cells (TM4 cells as described, e.g., in Mather, J.P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J.P. et al., Annals N.Y. Acad. Sci. 383 (1982) 44-68); MRC 5 cells; and FS4 cells. Other useful mammalian host cell lines include Chinese hamster ovary (CHO) cells, including DHFR- CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A.M., Methods in Molecular Biology, Vol. 248, Lo, B.K.C. (ed.), Humana Press, Totowa, NJ (2004), pp. 255-268.

In some aspects, the host cell is eukaryotic, e.g., a Chinese Hamster Ovary (CHO) cell or lymphoid cell (e.g., Y0, NS0, Sp20 cell).

### C. Assays

Anti-Notch2 antibodies provided herein may be identified, screened for, or characterized for their physical/chemical properties and/or biological activities by various assays known in the art.

### 1. Binding Assays and Other Assays

In some aspects, an antibody of the invention is tested for its antigen binding activity, e.g., by known methods such as ELISA, Western blot, etc.

In some aspects, competition assays may be used to identify an antibody that competes with one or more of antibodies rat.1B2 or a humanized version thereof, rat.3107, rb.2338, rb.2430, and/or rb.2621 provided herein for binding to Notch2. In certain aspects, such a competing antibody binds to the same epitope (e.g., a linear or a conformational epitope) that is bound by rat. 1B2 or a humanized version thereof, rat.3107, rb.2338, rb.2430, and/or rb.2621. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized Notch2 is incubated in a solution comprising a first labeled antibody that binds to Notch2 (e.g., rat.1B2 or a humanized version thereof, rat.3107, rb.2338, rb.2430, or rb.2621) and a second unlabeled antibody that is being tested for its ability to compete with the first antibody for binding to Notch2. The second antibody may be present in a hybridoma supernatant. As a control, immobilized Notch2 is incubated in a solution comprising the first labeled antibody but not the second unlabeled antibody. After incubation under conditions permissive for binding of the first antibody to Notch2, excess unbound antibody is removed, and the amount of label associated with immobilized Notch2 is measured. If the amount of label associated with immobilized Notch2 is substantially reduced in the test sample relative to the control sample, then that indicates that the second antibody is competing with the first antibody for binding to Notch2. See Harlow and Lane (1988) Antibodies: A Laboratory Manual ch.14 (Cold Spring Harbor Laboratory, Cold Spring Harbor, NY).

In an exemplary epitope binning assay, surface plasmon resonance is used to determine competition between antibodies. For example, a first antibody (e.g., rat.1B2 or a humanized version thereof, rat.3107, rb.2338, rb.2430, or rb.2621) is immobilized on an SPR sensorprism CMD 200M chip using amino coupling. Analyte is injected for 4 minutes, e.g., at 50 nM, and then a second antibody is injected for 4 minutes, e.g., at 10 µg/ml. The assay may be performed at 25° C. in a running buffer of HBS-T buffer (0.01 M HEPES pH 7.4, 0.15 M NaCl, 0.05% surfactant P20, 5 mM CaC1₂). The binning data may be processed using Wasatch binning software tool, Epitope (Carterra USA).

### 2. Activity Assays

In some aspects, assays are provided for identifying anti-Notch2 antibodies having a particular biological activity. For example, assays are provided for identifying anti-Notch2 antibodies that inhibit Jagged1-mediated signaling, but which leave DLL1-mediated signaling substantially intact. Assays are also provided for identifying anti-Notch2 antibodies that reduce the number of secretory cells in vitro and/or in vivo.

A nonlimiting exemplary assay for identifying anti-Notch2 antibodies that inhibit Jagged1-mediated signaling, but which leave DLL1-mediated signaling substantially intact is described in Example 5. Generally, in some embodiments, a test antibody is added to a culture of human cells that express human Notch2, such as cell line U87-MG. The culture is then contacted with cells that express Jagged1 or DLL1. Ligand-dependent Notch2 activation results in Notch2-ICD translocation in the Notch2-expressing cells. Following incubation, the co-cultured cells are fixed and permeabilized, and then contacted with an anti-Notch2 ICD antibody. After removing unbound anti-Notch2 ICD antibody, the bound antibody is detected, for example, using a labeled anti-Ig antibody. If the anti-Notch2 test antibody inhibits Jagged1-mediated signaling but not DLL1-mediated signaling, then the co-culture with cells expressing DLL1 will produce substantially greater signal than the co-culture with cells expressing Jagged1 will not.

In some embodiments, an anti-Notch2 antibody is assayed to determine if it reduces the number of secretory cells. A nonlimiting exemplary assay to select antibodies with this activity is described in Example 8. Generally, in some embodiments, an air-liquid interface (ALI) culture of primary human bronchial epithelial cells (HBECs) is established and cultured for several weeks until they are fully differentiated, as indicated for example, when cilia are visibly beating. Test anti-Notch2 antibody is added to the media in the lower chamber of the ALI culture. After about 7 days, the ALI cultures are analyzed. RNA is extracted from a sample of the culture and assayed for expression of genes indicative of secretory cells, such as Muc5b, Muc5ac and Scgb1a1. The cultures may also be analyzed by histology by fixing the cultures and embedding in paraffin. Sections are stained with antibodies to markers for secretory cells, such as Muc5b, and ciliated cells, such as tubulin. Cultures incubated with and without test anti-Notch2 antibody are compared to identify anti-Notch2 antibodies that reduce the number of secretory cells, such as goblet cells.

### D. Methods and Compositions for Diagnostics and Detection

In certain aspects, any of the anti-Notch2 antibodies provided herein is useful for detecting the presence of Notch2 in a biological sample. The term “detecting” as used herein encompasses quantitative or qualitative detection. In certain aspects, a biological sample comprises a biological fluid, cell, or tissue, such as sputum, secretory cells, airway epithelial cells, immune cells, lung cells or tissue, or bronchial cells or tissue.

In some aspects, an anti-Notch2 antibody for use in a method of diagnosis or detection is provided. In a further aspect, a method of detecting the presence of Notch2 in a biological sample is provided. In certain aspects, the method comprises contacting the biological sample with an anti-Notch2 antibody as described herein under conditions permissive for binding of the anti- Notch2 antibody to Notch2, and detecting whether a complex is formed between the anti-Notch2 antibody and Notch2. Such method may be an in vitro or in vivo method. In some aspects, an anti-Notch2 antibody is used to select subjects eligible for therapy with an anti-Notch2 antibody, e.g., where Notch2 is a biomarker for selection of patients.

In certain aspects, labeled anti-Notch2 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Compositions

In a further aspect, provided are pharmaceutical compositions comprising any of the antibodies provided herein, e.g., for use in any of the below therapeutic methods. In some aspects, a pharmaceutical composition comprises any of the antibodies provided herein and a pharmaceutically acceptable carrier. In some aspects, a pharmaceutical composition comprises any of the antibodies provided herein and at least one additional therapeutic agent, e.g., as described below.

Pharmaceutical compositions of an anti-Notch2 antibody as described herein are prepared by mixing such antibody having the desired degree of purity with one or more optional pharmaceutically acceptable carriers (Remington’s Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in U.S. Pat. Publication Nos. 2005/0260186 and 2006/0104968. In some aspects, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. For example, it may be desirable to further provide an agent that can reduce mucus viscoelasticity. In some embodiments, an additional therapeutic agent is selected from hypertonic saline, mannitol, pulmozyme, N-acetyl cysteine, cysteamine, and a bronchodilator. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington’s Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### F. Therapeutic Methods and Routes of Administration

Any of the anti-Notch2 antibodies provided herein may be used in therapeutic methods.

In some aspects, an anti-Notch2 antibody for use as a medicament is provided. In further aspects, an anti-Notch2 antibody for use in treating a muco-obstructive lung disease is provided. In certain aspects, an anti-Notch2 antibody for use in a method of treatment is provided. In certain aspects, the invention provides an anti-Notch2 antibody for use in a method of treating an individual having a muco-obstructive lung disease comprising administering to the individual an effective amount of the anti-Notch2 antibody. In one such aspect, the method further comprises administering to the individual an effective amount of at least one additional therapeutic agent (e.g., one, two, three, four, five, or six additional therapeutic agents), e.g., as described below. In further aspects, the invention provides an anti-Notch2 antibody for use in reducing the number of secretory cells, such as goblet cells, in an individual, such as in the lungs of an individual. In certain aspects, the invention provides an anti-Notch2 antibody for use in a method of reducing the number of secretory cells, such as goblet cells, in an individual, such as in the lungs of an individual, comprising administering to the individual an effective amount of the anti-Notch2 antibody to reduce the number of secretory cells, such as goblet cells, in an individual, such as in the lungs of an individual. By reducing the number of secretory cells, such as goblet cells, in the lungs, the production of mucus in the lungs is reduced and/or clearance or mucus is increased, thereby alleviating one or more symptoms of, for example, a muco-obstructive lung disease. In some embodiments, treatment with an anti-Notch2 antibody provided herein improves FEV1 (forced expiratory volume in one second), reduces breathlessness, and/or reduces cough in a subject with a muco-obstructive lung disease.

In a further aspect, the invention provides for the use of an anti-Notch2 antibody in the manufacture or preparation of a medicament. In some aspects, the medicament is for treatment of a muco-obstructive lung disease. In a further aspect, the medicament is for use in a method of treating a muco-obstructive lung disease comprising administering to an individual having a muco-obstructive lung disease an effective amount of the medicament. In one such aspect, the method further comprises administering to the individual an effective amount of at least one additional therapeutic agent, e.g., as described below. In a further aspect, the medicament is for reducing the number of secretory cells, such as goblet cells, in an individual, such as in the lungs of an individual. In a further aspect, the medicament is for use in a method of reducing the number of secretory cells, such as goblet cells, in an individual, such as in the lungs of an individual, comprising administering to the individual an effective amount of the medicament to reducing the number of secretory cells, such as goblet cells, in an individual, such as in the lungs of an individual.

In a further aspect, the invention provides a method for treating a muco-obstructive lung disease. In some aspects, the method comprises administering to an individual having such muco-obstructive lung disease an effective amount of an anti-Notch2 antibody. In one such aspect, the method further comprises administering to the individual an effective amount of at least one additional therapeutic agent, as described below.

In a further aspect, the invention provides a method for reducing the number of secretory cells, such as goblet cells, in an individual, such as in the lungs of an individual. In some aspects, the method comprises administering to the individual an effective amount of an anti-Notch2 antibody to reducing the number of secretory cells, such as goblet cells, in an individual, such as in the lungs of an individual. In some aspects, an “individual” is a human.

Nonlimiting exemplary muco-obstructive lung diseases that may be treated with the anti-Notch2 antibodies provided herein include chronic obstructive lung disease (COPD), cystic fibrosis, primary ciliary dyskinesia, non-cystic fibrosis bronchiectasis, and bronchiolitis.

An “individual” or “subject” according to any of the above aspects may be a human.

In a further aspect, the invention provides pharmaceutical compositions comprising any of the anti-Notch2 antibodies provided herein, e.g., for use in any of the above therapeutic methods. In some aspects, a pharmaceutical composition comprises any of the anti-Notch2 antibodies provided herein and a pharmaceutically acceptable carrier. In some aspects, a pharmaceutical composition comprises any of the anti-Notch2 antibodies provided herein and at least one additional therapeutic agent, e.g., as described below.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents). In certain aspects, the combination therapy comprises administering an antibody of the invention and administering at least one additional therapeutic agent, such as an agent that reduces mucus viscoelasticity. In some embodiments, an additional therapeutic agent is selected from hypertonic saline, mannitol, pulmozyme, N-acetyl cysteine, cysteamine, and a bronchodilator.

Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents. In some aspects, administration of the anti-Notch2 antibody and administration of an additional therapeutic agent occur within about one month, or within about one, two or three weeks, or within about one, two, three, four, five, or six days, of each other. In some aspects, the antibody and additional therapeutic agent are administered to the patient on Day 1 of the treatment.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, or by intrapulmonary (e.g., inhalation) or intranasal delivery, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient’s clinical history and response to the antibody, and the discretion of the attending physician. The antibody is suitably administered to the patient at one time or over a series of treatments. Such doses may be administered intermittently, e.g., every week or every three weeks (e.g., such that the patient receives from about two to about twenty, or, e.g., about six doses of the antibody). An initial higher loading dose, followed by one or more lower doses may be administered. However, other dosage regimens may be useful. The progress of this therapy is easily monitored by conventional techniques and assays.

### G. Articles of Manufacture

In some aspects of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

### III. EXAMPLES

The following are examples of methods and compositions of the invention. It is understood that various other embodiments may be practiced, given the general description provided above.

### Example 1: Generation of Rabbit and Rat Anti-Notch2 Antibodies

New Zealand White rabbits were co-immunized with human and murine extracellular domain (ECD) constructs comprising EGF repeats 6-10 of Notch2 (huNotch2-EGF6-10 and muNotch2-EGF6-10) and single B cells were isolated using a modified protocol based on Offner et al. PLoS ONE 9(2), 2014. The modified workflow included direct FACS sorting of IgG+ huNotch2+ B cells into single wells. The B cell culture supernatants were assayed by ELISA for binding to human Notch2 and an irrelevant control protein. Notch2 specific B cells were lysed and immediately frozen in -80° C. for storage until molecular cloning. Variable regions (VH and VL) of each monoclonal antibody from rabbit B cells were cloned into expression vectors with human constant region with N297G mutation from extracted mRNA as previously described (Offner et al. PLoS ONE 9(2), 2014). Individual recombinant chimeric rabbit/human antibodies were expressed in Expi293 cells and subsequently purified with protein A. Purified anti-Notch2 antibodies were then subjected to functional activity assays and kinetic screening, as described herein.

Rats were immunized with either a combination of MBP-huNotch2 EGF6-10 + MBP-huNotch2 EGF7-9 or primed with MBP-huNotch2 EGF6-10 and boosted with huNotch2-EGF6-10 and hybridomas were generated using a modified fusion partner (Price et al. J Immunol Methods 2009). Various conditions were optimized to enable sorting of individual IgG+ huNotch2+ hybridomas into single wells followed by additional culturing after sorting. The resulting hybridoma supernatants were assayed by ELISA and positive samples were purified using protein A for subsequent functional and kinetic characterization. Certain rat monoclonal antibodies were sequenced and cloned into a constant region with N297G mutation. Individual recombinant chimeric rat/human antibodies were expressed in Expi293 cells and subsequently purified with protein A. Purified anti-Notch2 antibodies were then subjected to functional activity assays and kinetic screening, as described herein.

### Example 2: Kinetic Analysis and Epitope Binning Using Array-Based Surface Plasmon Resonance

An array-based SPR imaging system (Carterra USA) was used to epitope bin a panel of five monoclonal antibodies generated in Example 1 (rat.1B2, rat.3107, rb.2338, rb.2430, and rb.2621), as well as anti-Notch ⅔ antibody OMP-59R5 (tarextumab, see U.S. Pat. No. 8,226,943 B2). Purified antibodies were diluted at 10 µg/ml in 10 mM sodium acetate buffer pH 4.5. Using amine coupling, antibodies were directly immobilized onto a SPR sensorprism CMD 200M chip (XanTec Bioanalytics, Germany) using a Continuous Flow Microspotter (Carterra, USA) to create an array of six antibodies. For analysis, the IBIS MX96 SPRi (Carterra USA) was used to evaluate analytes binding to the immobilized ligands. For kinetic analyses, human Notch2 was injected for 3 minutes from 0 to 300 nM at 3-fold dilution followed by a dissociation period of 10 minutes. For epitope binning, human Notch2 was first injected for 4 minutes at 50 nM and was followed by a second 4 minute injection of individual monoclonal antibody at 10 µg/ml. The surface was regenerated with 10 mM glycine pH1.5 between cycles. The experiment was performed at 25° C. in a running buffer of HBS-T buffer (0.01 M HEPES pH 7.4, 0.15 M NaC1, 0.05% surfactant P20, 5 mM CaCl₂). The binning data was processed using Wasatch binning software tool, Epitope (Carterra USA).

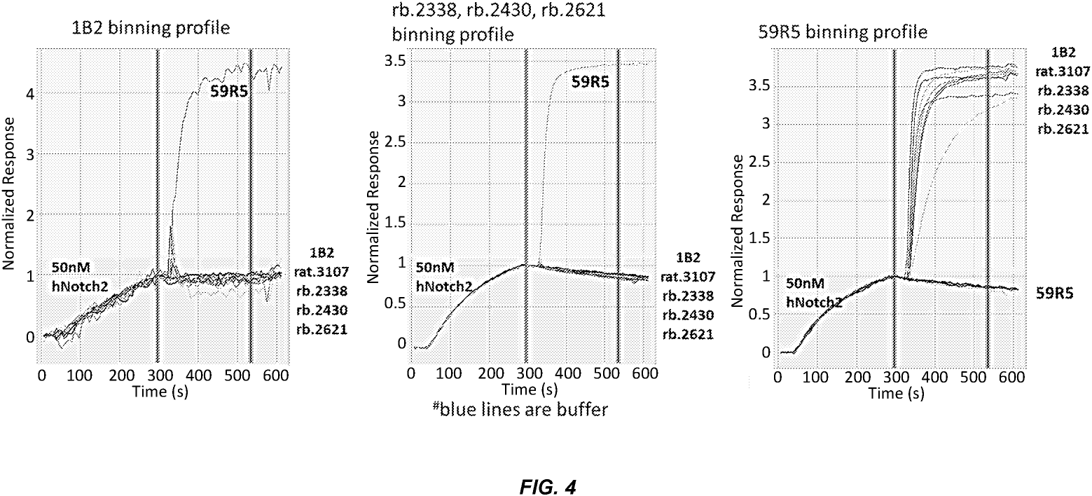

The results are shown in FIG. 4. Antibodies rat. 1B2, rat.3107, rb.2338, rb.2430, and rb.2621 were determined to be in a different epitope bin from anti-Notch ⅔ antibody OMP-59R5.

### Example 3: Humanization of Rat Anti-Notch2 Antibodies

Rat monoclonal antibodies 1B2 and 3107 were humanized as described below. Residue numbers are according to Kabat et al., Sequences of proteins of immunological interest, 5th Ed., Public Health Service, National Institutes of Health, Bethesda, Md. (1991).

Variants constructed during the humanization of 1B2 and 3107 were assessed in the form of human IgG. Hypervariable regions from each of the antibodies (positions 24-34 (L1), 50-56 (L2) and 89-97 (L3) in VL domain, and 26-35 (H1), 50-65 (H2) and 95-102 (H3) in VH domain) were grafted into various acceptor frameworks. For rat 1B2, VL CDRs were grafted into KV1-12*01, and VH CDRs were grafted into HV3-73*01. In addition, a glycosylation site in CDR-H2 Asn54-Phe55-Ser56 was mutated to Asp54-Phe55-Ser56. For rat 3107, VL CDRs were grafted into KV2-30*02 and VH CDRs were grafted into HV1-2*01. All VL and VH Vernier positions from parental antibodies were also grafted into their respective human germline frameworks. The grafts with all rat amino acids in Vernier positions are referred to as L1H1 (hu.1B2.L1H1 and hu.3107.L1H1).

The binding affinity of hu.1B2.L1H1 antibody was compared to its chimeric parental clone. Rat Vernier positions of version L1H1 antibodies were converted back to human residues to evaluate the contribution of each rat Vernier positions to binding affinity to huNOTCH2. Four additional light chain Vernier variants L2-L5, and eight additional heavy chain Vernier variants H2-H9 were made. Ser43 and Tyr71 on the light chain (L7), and Va124, Ala49, Ser76, and Leu78 on the heavy chain (H14) were determined to be the key rat Vernier residues based on binding affinity evaluation of the variant antibodies described above (data not shown). Binding affinity was determined as discussed below in Example 6. Chimeric 1B2 bound with a K_{D} of 5.21E-9 M, while hu1B2.L7H14 bound with a K_{D} of 6.13E-9 M.

The binding affinity of hu.3107.L1H1 antibody was compared to its chimeric parental clone. Rat Vernier positions of version L1H1 antibodies were converted back to human residues to evaluate the contribution of each rat Vernier positions to binding affinity to huNOTCH2. One additional light chain variant (L2) and ten additional heavy chain variants H2-H11 were made.

To increase the affinity of anti-Notch2 humanized antibodies based on 3107, 4 heavy chain sequence variants were made based on binding affinity evaluation and HCS potency of the humanized antibodies (data not shown): H12 in HV1-2*01 with P45, T48, A67, V71, S75 and T76; H13 in HV1-2*01 with P45, T48, A67, V71, T76; H14 and H15 are in HV5-51*01 with the same Vernier residues as H12 and H13 respectively. For the light chain, germline KV4-1*1 was used for CDR graft (L7). In addition, V2 and F36 on the light chain were determined to be rat Vernier residues that maintain potency in the HCS assay and were grafted onto Germline KV4-1*01 (L6). The HCS assays were carried out substantially as described in Example 5.

### Example 4: Affinity Improvement of Humanized 1B2 Antibodies

To increase the potency of anti-Notch2 humanized antibodies based on 1B2, 560 single point mutation variants were generated using L7H10 as template. The resulting antibodies were screened by surface plasmon resonance and ranked according to off-rates. There were five mutations in the heavy chain (A50G, S51Q, I57R, S96H, and R98F) and three mutations in the light chain (S31V, Q55H, and L96I) that resulted in a slower off-rate. To identify good combinations of mutations, 80 variants were generated with individual and combined sets of mutations and evaluated by surface plasmon resonance characterization. S51Q was identified as a mutation that improved off-rate.

In order to further improve the affinity of 1B2, L1H1 with S51Q and N54D mutations was used as a template for phage display affinity maturation. Briefly, a total of four phage libraries were constructed and displayed as monovalent Fab on the surface of M13 bacteriophage. The first set of libraries consisted of two CDRs NNK walk (one for CDR-H1, H2, and H3, and one for CDR-L1, L2, and L3) where one position in each of the three CDRs was randomized simultaneously. The second set consisted of two hard randomization libraries where the entire CDR-L3 or CDR-H3 were mutated.

For affinity improvement selection, phage libraries were subjected to four rounds of solution sorting with increasing stringency and cold human Notch2 EGF6-10 as competitor. Enrichment was observed for the CDR-H3 hard randomized library. After comparing the parent sequence to enriched clones, several CDR-H3 mutations were identified. A total of 54 combination variants were reformatted into human IgG1 for antibody production and further BIAcore binding kinetic analysis and HCS assay. The HCS assays were carried out substantially as described in Example 5. hu1B2.v2, hu1B2.v4, hu1B2.v9, and hu1B2.v8 were identified as the most improved in both affinity and potency in HCS assay. The CDR-H3 of these four variants were grafted into Vernier polished-humanization variant L7H14 to generate hu1B2.v101, hu1B2.v102, hu1B2.v103, and hu1B2.v104 respectively. Binding affinity was determined as discussed below in Example 6. hu1B2.L7H14 has an affinity of 6.13E-9M for hu.Notch2 while hu1B2.v101, hu1B2.v102, hu1B2.v103, and hu1B2.v014 have affinities of 2.84E-09, 3.37E-09, 3.08E-09, and 3.09E-09, respectively. None of the variants showed binding to human Notch1, human Notch3 or human Notch4 by surface plasmon resonance. Non-specific binding of each anti-Notch2 variant was measured in an ELISA with baculovirus particles (Hotzel et al. MAbs 2012). Hu1B2.v102 and hu1B2.v104 were assayed for molecular assessment liabilities using thermal stress and AAPH oxidation stress tests (see Dion et al. J. Pharm. Sci 2018, 107(2), 550). No liabilities were identified.

### Example 5: High-Content Screening (HCS) Assay to Identify Antibodies That Block Jagged1 Signaling But Not DLL1 Signaling

Human cell line U87-MG, which expresses high levels of huNotch2 (N2) endogenously, was harvested and 4,000 cells per well were seeded onto Cell Carrier ultra 384-well plates (Perkin Elmer, Waltham, MA). The plates were cultured at 37° C. CO₂-incubator for 2-5 hrs, and during this incubation time, antibody (Ab) test samples were prepared with initial dilutions manually then followed by a set of 10 points of 3 or 3.5-fold serial dilutions carried out by Bravo automated liquid handler (Agilent, Santa Clara, CA). Diluted Ab samples were transferred to a duplicate set of plates containing U-87-MG cells. After addition of diluted Abs, the 3T3-Jag1 or OP9-DLL1-cells were harvested and each ligand cell line was seeded at 4,000 cells per well on top of the U-87-MG cells treated with Ab and incubated to allow the ligand-dependent Notch-2 activation and N2-ICD translocation to occur in U-87-MG cells.

After 16 to 22 hr incubation, each co-culture of receptor and ligand-expressing cells was fixed with 4% paraformaldehyde for 10 minutes, plates were washed with PBS and then cells were permeabilized by 0.05% Saponin (Sigma-Aldrich, San Louis, MO) in PBS+ 0.05 % BSA buffer for 1 hr. After permeabilization, the plates were washed and rabbit anti-N2-ICD mAb D76A6 (Cell Signaling Technology, Danvers, MA) was diluted with 0.05% Saponin containing PBS/BSA buffer and added onto the plates and incubated at 4° C. overnight.

The next day, plates were washed and stained with buffer containing detection AF-647 conjugated anti-rabbit detection Ab (Jackson-Immunoresearch, West Grove, PA) and Hoechst-33342 dye (Thermo Fisher Scientific, Waltham, MA), then incubated at room temperature for 2 hrs with gentle shaking. After cells were stained, the plates were washed with wash buffer and then PBS was added into each well and then plates were imaged.

Six images were taken from each well using a 20X water immersion objective on an Opera Phenix High Content imaging system (Perkin Elmer, Waltham, MA). Analysis was performed using Columbus software imaging analysis tool (Perkin Elmer, Waltham, MA), where the nuclear area and ring area surrounding the nuclei were identified and the signal intensities were calculated. A threshold was obtained in order to calculate the N2-ICD nuclei translocation positive population from the maximal inhibitory control samples. The results from Columbus software analysis were uploaded into Genedata Screener application (Lexington, MA), where a normalization process was set up using the translocation percentage derived from neutral controls subtracted by maximal inhibition control and the IC50 values calculated.

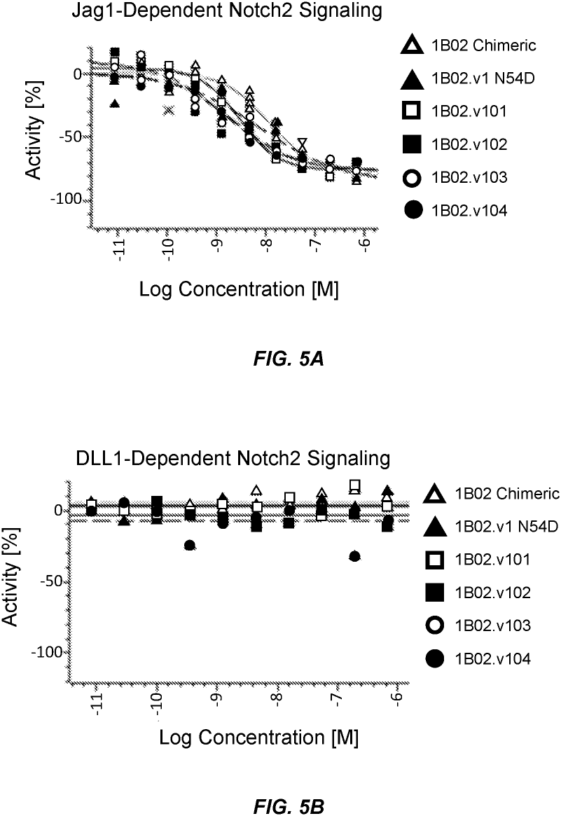

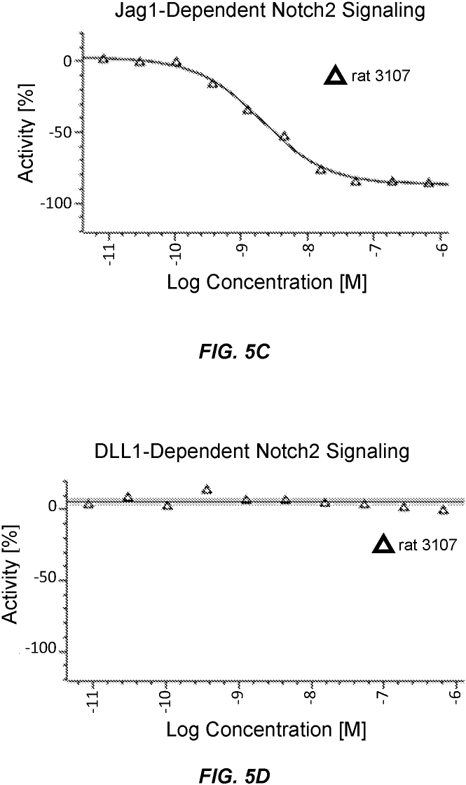

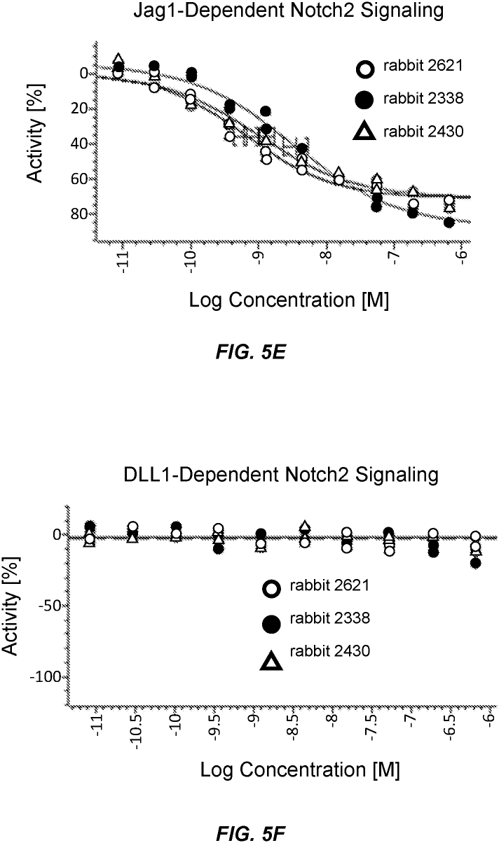

Data analysis results from 3T3-Jag1 and OP9-DLL1 co-culture sets were compared and used for the discovery of Notch2 antibodies that block Jagged1-mediated activation but spare DLL1-mediated activation, and optimization of humanized versions of the antibodies. Exemplary results are shown in FIGS. 5A-5F. All of the tested antibodies blocked Jagged1-mediated activation but spared DLL1-mediated activation. Table 2 summarizes the IC50s of each antibody for blocking Jagged1-mediated signaling.

TABLE 2

Jagged1 IC50s of anti-Notch2 antibodies

| | |
|---|---|
| Compound ID | Jagged1 IC50 [M] |
| 1B2 Chimeric | 1.253E-8 |
| hu1B2.L1H1.DFS | 7.896E-9 |
| hu1B2.v101 | 3.017E-9 |
| hu1B2.v102 | 1.591E-9 |
| hu1B2.v103 | 1.801E-9 |
| hu1B2.v104 | 2.485E-9 |
| rat 3107 | 2.101E-9 |
| rabbit 2621 | 6.671E-10 |
| rabbit 2338 | 3.530E-9 |
| rabbit 2430 | 1.027E-9 |

In a separate experiment, rat antibody 3107 and humanized versions of 3107 were tested in the HCS assay substantially as described above. All antibodies tested in this experiment comprised human IgG1 with an N297G mutation. 3107 and the humanized variants all blocked Jagged1-mediated activation but spared DLL1-mediated activation (data not shown). Table 3 summarizes the IC50s of each antibody for blocking Jagged1-mediated signaling. Table 3: Jagged1 IC50s of anti-Notch2 3107 antibody and humanized variants

| | |
|---|---|
| Compound ID | Jagged1 IC50 [M] |
| Rat 3107 | 4.88E-09 |
| hu.Notch-3107.L1H15 | 6.08E-09 |
| hu.Notch-3107.L7H12 | 1.10E-08 |
| hu.Notch-3107.L7H13 | 5.94E-09 |
| hu.Notch-3107.L7H14 | 1.05E-08 |
| hu.Notch-3107.L6H12 | 4.71E-09 |
| hu.Notch-3107.L6H13 | 6.42E-09 |
| hu.Notch-3107.L7H15 | 9.16E-09 |
| hu.Notch-3107.L6H14 | 6.65E-09 |
| hu.Notch-3107.L1H12 | 7.85E-09 |
| hu.Notch-3107.L1H13 | 4.92E-09 |
| hu.Notch-3107.L1H14 | 6.95E-09 |
| hu.Notch-3107.L6H15 | 8.03E-09 |

### Example 6: Kinetic Analyses Using BIAcore™

The binding affinities of the antibodies was determined by BIAcore™ T200 machine. Rabbit antibodies were expressed as chimeric antibodies with rabbit variable domains and human constant domains. Rat antibodies were expressed as chimeric antibodies with rat variable domains and human constant regions. Humanized antibodies were expressed in the human IgG1 backbone. For kinetics measurements, antibodies were captured on research grade protein A chip (GE Healthcare) to achieve approximately 300 RU. Ten-fold serial dilutions of huNotch2-EGF6-10 were injected in HBS-P buffer with additional 3 mM CaC1₂ at 37° C. with a flow rate of 100 µL/min. Association rates (ka) and dissociation rates (kd) were calculated using a 1:1 Langmuir binding model (BIAcore™ T200 Evaluation Software version 2.0). The equilibrium dissociation constant (KD) was calculated as the ratio kd/ka. The results are shown in Table 4.

TABLE 4

Binding properties of rat.3107, rat.1B2 and certain humanized variants, and rb.2338, rb.2430, and rb.2621 to huNotch2-EGF6-10 (n=3)

| | | | |
|---|---|---|---|
| Sample | ka (⅟Ms) | kd (⅟s) | K_{D} (M) |
| Rat-1B2 | 1.81E+05 | 9.43E-04 | 5.21E-09 |
| hulB2.L1H1.DFS | 2.26E+05 | 1.16E-03 | 5.15E-09 |
| hu1B2.L7H10 | 4.18E+05 | 4.15E-03 | 9.94E-09 |
| hu1B2.L7H14 | 2.19E+05 | 1.34E-03 | 6.13E-09 |
| hu1B2.v101 | 2.86E+05 | 8.13E-04 | 2.84E-09 |
| hu1B2.v102 | 3.87E+05 | 1.31E-03 | 3.37E-09 |
| hu1B2.v103 | 2.65E+05 | 8.17E-04 | 3.08E-09 |
| hu1B2.v104 | 3.57E+05 | 1.10E-03 | 3.09E-09 |
| Rat-3107 | 3.63E+05 | 1.82E-03 | 5.00E-09 |
| Rabbit 2338 | 5.33E+05 | 6.01E-03 | 11.3E-09 |
| Rabbit 2430 | 2.19E+06 | 3.07E-02 | 14.0E-09 |
| Rabbit 2621 | 2.48E+06 | 3.05E-02 | 12.3E-09 |

In a separate experiment, the binding affinities of the humanized versions of the rat 3107 antibody were determined, substantially as described above. The results are shown in Table 5.

TABLE 5

Binding properties of rat.3107 and certain humanized variants

| | | | |
|---|---|---|---|
| Sample | ka (⅟Ms) | kd (⅟s) | KD (M) |
| rat.3107 | 3.42E+05 | 3.12E-03 | 9.12E-09 |
| hu.3107.L1H12 | 3.80E+05 | 5.42E-03 | 1.43E-08 |
| hu.3107.L1H13 | 3.79E+05 | 5.87E-03 | 1.55E-08 |
| hu.3107.L1H14 | 3.60E+05 | 6.31E-03 | 1.75E-08 |
| hu.3107.L1H15 | 3.55E+05 | 6.34E-03 | 1.79E-08 |
| hu.3107.L6H12 | 4.13E+05 | 4.04E-03 | 9.79E-09 |
| hu.3107.L6H13 | 4.48E+05 | 4.24E-03 | 9.47E-09 |
| hu.3107.L6H14 | 3.54E+05 | 3.54E-03 | 1.00E-08 |
| hu.3107.L6H15 | 3.96E+05 | 3.65E-03 | 9.23E-09 |
| hu.3107.L7H12 | 3.84E+05 | 3.67E-03 | 9.54E-09 |
| hu.3107.L7H13 | 3.55E+05 | 3.53E-03 | 9.94E-09 |
| hu.3107.L7H14 | 3.46E+05 | 3.59E-03 | 1.04E-08 |
| hu.3107.L7H15 | 3.50E+05 | 4.07E-03 | 1.16E-08 |

Binding of the anti-Notch2 antibodies to Notch2 from additional species and to constructs comprising different EGF repeat regions was evaluated by BIAcore™. For this experiment, the antibodies with human constant regions were captured on a protein A chip to achieve approximately 200 RU. Ten-fold serial dilutions of various antigens were injected in HBS-P buffer with additional 3 mM CaC1₂ at 37° C. with a flow rate of 100 µL/min. The results of the experiment are summarized in Table 6.

TABLE 6

Binding of rat.3107, certain rat.1B2 humanized versions, and rb.2338, rb.2430, and rb.2621 to various Notch2 constructs, human Notch1, and human Notch3

| | | | | | | |
|---|---|---|---|---|---|---|
| | hu.1B2.v102 | hu1B2.v104 | rat.3107 | rb.2338 | rb.2430 | Rb.2621 |
| huNotch2-EGF6-10 | + | + | + | + | + | + |
| huNotch2-EGF4-7 | + | + | + | nt | nt | nt |
| huNotch2-EGF5-8 | + | + | + | nt | nt | nt |
| huNotch2-EGF7-9 | + | + | + | nt | nt | nt |
| huNotch2-EGF6-12.R268K | - | - | - | +/ | +/ | +/ |
| muNotch2-EGF6-10 | - | - | - | - | - | - |
| muNotch2-EGF6-12.K268R | + | + | + | + | + | + |
| gpNotch2-EGF6-12 | + | + | + | + | + | + |
| ratNotch2-EGF6-10 | - | - | - | nt | nt | nt |
| huNotch1 | - | - | - | nt | nt | nt |
| huNotch3 | - | - | - | - | - | - |

nt = not tested.

Other humanized versions of 1B2 (hu1B2.L1H1.DFS, hu1B2.v4L7, hu1B2.v8L7, hu1B2.v9L7, hu.1B2.DFS.H14L7) showed similar binding profiles as hu.1B2.v102 and hu.1B2.v104 in Table 4, above. Based on the binding characteristics of the anti-Notch2 antibodies shown in Table 4, rat.3107, humanized versions of rat.1B2, rb.2338, rb.2430, and rb.2621 bind an epitope within human Notch2 EGF7. Further, all of the antibodies tested show little or no binding to huNotch2-EGF6-12.R268K or to muNotch2-EGF6-10, but bind to huNotch2-EGF6-10 and muNotch2-EGF6-12.K268R, suggesting that the antibodies contact arginine at position 268 of human Notch2.

### Example 7: Inhibition of Jagged1 and DLL1 Signaling by Anti-Notch2 Fabs

Certain anti-Notch2 antibodies were reformatted as monovalent Fabs, and assayed for Jagged1 and DLL1 signaling inhibition using the HCS assay described in Example 5.

Data analysis results from 3T3-Jag1 and OP9-DLL1 co-culture sets were used to calculate the Jagged1 IC50, and determine the maximum percent inhibition of Jagged1 and DLL1 signaling by the Fabs. Table 7 shows the maximum Jagged1 and DLL1 signaling inhibition observed for each Fab.

TABLE 7

Maximum inhibition by anti-Notch2 Fabs

| Fab | Maximum Jagged1 inhibition | Maximum DLL1 inhibition |
|---|---|---|
| hu1B2.v8 | 100% | 50% |
| hu1B2.v104 | 100% | 60% |

Surprisingly, while both hu1B2.v8 and hu1B2.v104 were selective for inhibition of Jagged1 signaling in a bivalent antibody format, when reformatted as monovalent Fabs, both hu1B2.v8 and hu1B2.v104 inhibited DLL1 signaling, although to a reduced maximum inhibition compared to inhibition of Jagged1 signaling. In contrast, monovalent Fab-formatted hulB2.v1.DFS, hu1B2.v101, and hu1B2.v103 retained Jagged1-specific signaling inhibition activity, and did not inhibit DLL1 (data not shown). Without intending to be bound by any particular theory, the difference in selectivity between Fab-formatted hu1B2.v8 and hu1B2.v104 and Fab-formatted hulB2.v1.DFS, hu1B2.v101, and hu1B2.v103 may be attributable to the difference in CDR-H3 sequences. Hu1B2.v8 and hu1B2.v104 share the CDR-H3 sequence DGGKLALDA (SEQ ID NO: 11), while hulB2.v1.DFS, hu1B2.v101, and hu1B2.v103 have the CDR-H3 sequences DSGRWGLDA (SEQ ID NO: 8), DGGRWGLDA (SEQ ID NO: 9), and DGGKWGLDA (SEQ ID NO: 12), respectively.

### Example 8: Reduction of Secretory Cells by Anti-Notch2 Antibodies

Air-liquid interface (ALI) cultures: Primary human bronchial epithelial cells (HBECs) are plated in 0.4 µm-pore PET transwells (Corning #7369) and cultured under submerged conditions until confluent in Pneumacult Ex-Plus media (StemCell Technologies #05040). Once confluent, media from the upper chamber is removed exposing the HBECs to air and the media in the lower chamber is replaced with Pneumacult ALI basal media (StemCell Technologies #05001). Cells are cultured for 3-4 weeks and are fully differentiated when cilia are visibly beating.

Antibody treatment and sample analysis: Antibodies were added to the basal media at a concentration of 50 mg/ml. As media was replaced in the lower chamber (3x a week), the antibody was replenished. At Day 7, the ALI cultures were collected for RNA analysis and histology. For RNA analysis, RNA was extracted using Qiagen RNA Extraction kit (#74106). Following cDNA synthesis using iScript cDNA synthesis (Biorad #1708891), gene expression analysis was performed for genes Muc5b, Muc5ac and Scgb1a1 (Taqman Assays). For histology analysis, transwells were formalin fixed and paraffin embedded. Samples were sectioned and stained for anti-Muc5b (goblet cells), anti-acetylated a-tubulin (ciliated cells) and DAPI (nuclear staining).

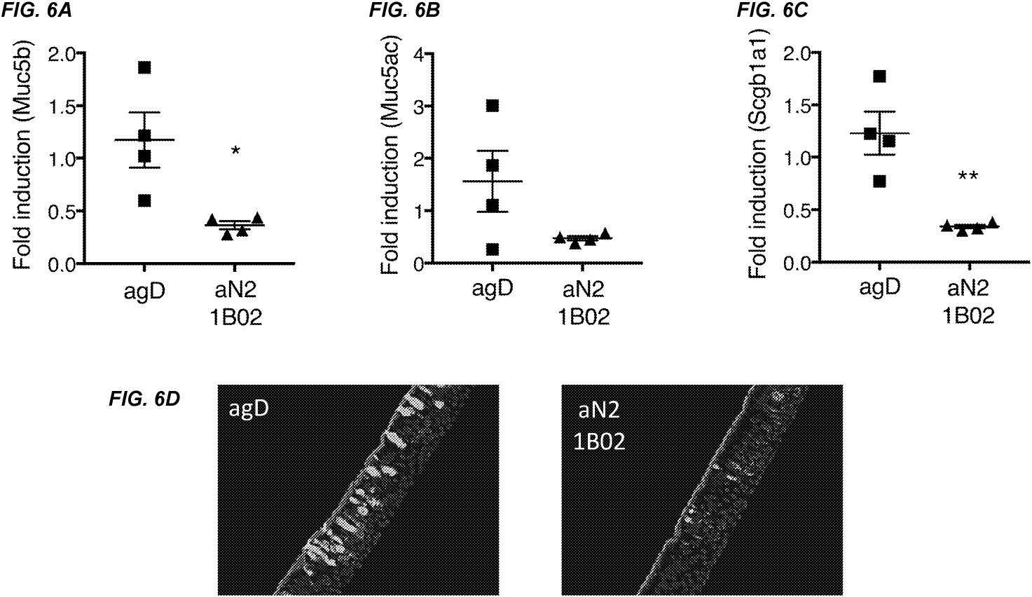

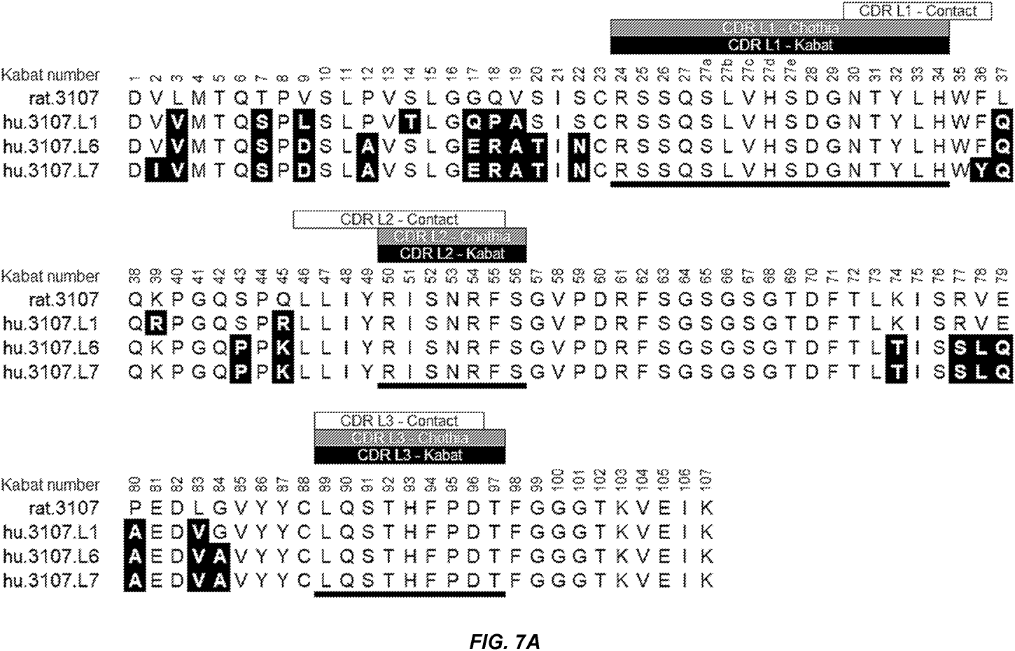

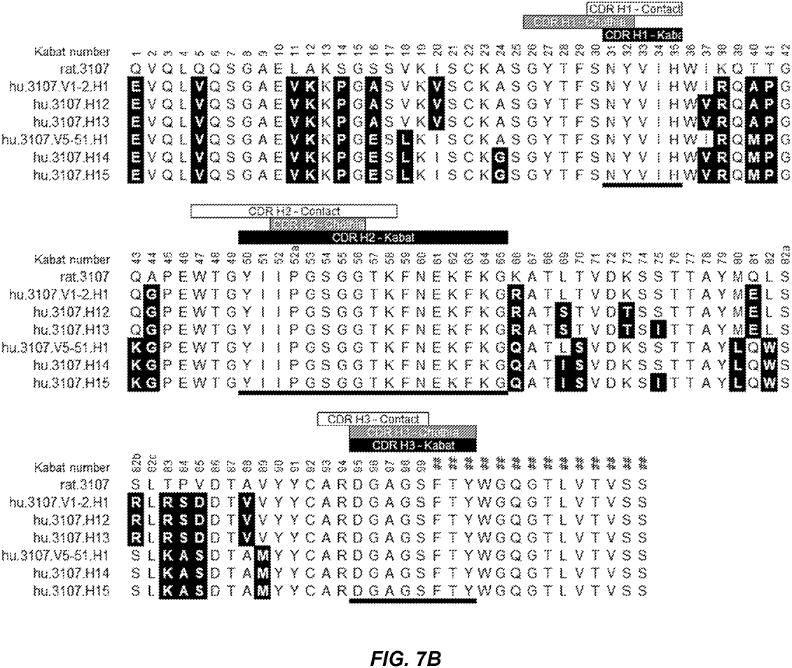

As shown in FIGS. 6A-6D, treatment with anti-Notch2 antibody 1B2 reduced Muc5b, Muc5ac, and Scgb1a1 mRNA expression in ALI cultures of HBECs. Treatment with anti-Notch2 antibody 1B2 also reduced the appearance of goblet cells, as detected by immunofluorescence using anti-Muc5b antibodies. These results show that inhibition of Jagged-Notch2 signaling is sufficient to significantly reduce secretory goblet cells in the culture.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### IV. TABLE OF CERTAIN SEQUENCES

| SEQ ID NO | Description | Sequence |
|---|---|---|
| 1 | CDR-L1 of rat1B2, hu1B2.L1, hu1B2.L7, hu1B2.v101, hu1B2.v102, hu1B2.v103, hu1B2.v104 | QTSEDIYSGLA |
| 2 | CDR-L2 of rat1B2, hu1B2.L1, hu1B2.L7, hu1B2.v101, hu1B2.v102, hu1B2.v103, hu1B2.v104 | GASRLQD |
| 3 | CDR-L3 of rat1B2, hu1B2.L1, hu1B2.L7, hu1B2.v101, hu1B2.v102, hu1B2.v103, hu1B2.v104 | QQGFKYPLT |
| 4 | CDR-H1 of rat1B2, hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104 | DFYME |
| 5 | CDR-H2 of rat1B2 | ASRNKANNFSIVYSASVKD |
| 6 | CDR-H2 of hu.1B2.v1.DFS.H1, hu.1B2.H10, hu.1B2.DFS.H14 | ASRNKANDFSIVYSASVKD |
| 7 | CDR-H2 of hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104 | AQRNKANDFSIVYSASVKD |
| 8 | CDR-H3 of rat1B2, hu.1B2.v1.DFS.H1, hu.1B2.H10, hu.1B2.DFS.H14, hu.1B2.H1.N54D.S51Q | DSGRWGLDA |
| 9 | CDR-H3 of hu.1B2.v2, hu1B2.v101 | DGGRWGLDA |
| 10 | CDR-H3 of hu.1B2.v4, hu 1B2.v102 | DGGRLALDA |
| 11 | CDR-H3 of hu.1B2.v8, hu1B2.v104 | DGGKLALDA |
| 12 | CDR-H3 of hu.1B2.v9, hu1B2.v103 | DGGKWGLDA |
| 87 | LC-FR1 of hu1B2.L1, hu1B2.L7, hu.1B2.v101, hu.1B2.v102, hu.1B2.v103, hu.1B2.v104 | DIQMTQSPSS VSASVGDRVT ITC |
| 88 | LC-FR2 of hu1B2.L1, hu1B2.L7, hu.1B2.v101, hu.1B2.v102, hu.1B2.v103, hu.1B2.v104 | WYQQKP GKSPKLLIY |
| 89 | LC-FR3 of hu1B2.L1 | GVPS RFSGSGSGTD YTLTISSLQP EDFATYFC |
| 90 | LC-FR3 of hu1B2.L7, hu.1B2.v101, hu.1B2.v102, hu.1B2.v103, hu.1B2.v104 | GVPS RFSGSGSGTD YTLTISSLQP EDFATYYC |
| 91 | LC-FR4 of hulB2.L1, hu1B2.L7, hu.1B2.v101, hu.1B2.v102, hu.1B2.v103, hu.1B2.v104 | FGG GTKVEIK |
| 92 | HC-FR1 of hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104 | EVQLVESGGG LVQPGGSLKL SCAVSGFTFS |
| 93 | HC-FR2 of hu.1B2.v1.DFS.H1, hu.1B2.H1.N54D.S51Q, hu.1B2.v2, hu.1B2.v4, hu.1B2.v8, hu.1B2.v9, | WIRQA SGKGLEWIA |
| 94 | HC-FR2 of hu.1B2H10, hu.1B2.DFS.H14, hu1B2.v101, hu1B2.v102, hu1B2.v103, hu1B2.v104 | WVRQA SGKGLEWVA |
| 95 | HC-FR3 of hu.1B2.v1.DFS.H1, hu.1B2.H1.N54D.S51Q, hu.1B2.v2, hu.1B2.v4, hu.1B2.v8, hu.1B2.v9 | RF TISRDTSKST LYLQMNSLKT EDTAVYYCSR |
| 107 | HC-FR3 of hu.1B2.H10 | RF TISRDDSKST AYLQMNSLKT EDTAVYYCSR |
| 96 | HC-FR3 of hu.1B2.DFS.H14, hu1B2.v101, hu1B2.v102, hu1B2.v103, hu1B2.v104 | RF TISRDDSKST LYLQMNSLKT EDTAVYYCSR |
| 97 | HC-FR4 of hu.1B2.v1.DFS.H1, hu.1B2.H10, hu.1B2.DFS.H14, hu.1B2.H1.N54D.S51Q, hu.1B2.v2, hu.1B2.v4, hu.1B2.v8, hu.1B2.v9, hu1B2.v101, hu1B2.v102, hu1B2.v103, hu1B2.v104 | W GQGTLVTVSS |
| 13 | rat.1B2 Light Chain Variable Region (VL) | DIQMTQSPAS ASRLQDGVPS GTKLEIK |
| 14 | rat.1B2 Heavy Chain Variable Region (VH) | EVKLVDYGGG SRNKANNFSI DSGRWGLDAW |
| 15 | hu.1B2.L1 VL | DIQMTQSPSS ASRLQDGVPS GTKVEIK |
| 16 | hu.1B2.L7 VL | DIQMTQSPSS ASRLQDGVPS GTKVEIK |
| 17 | hu.1B2.v1.DFS.H1 VH | EVQLVESGGG SRNKANDFSI DSGRWGLDAW |
| 18 | hu.1B2.H10 VH | EVQLVESGGG SRNKANDFSI DSGRWGLDAW |
| 19 | hu.1B2.DFS.H14 VH | EVQLVESGGG SRNKANDFSI DSGRWGLDAW |
| 20 | hu.1B2.H1.N54D.S51Q VH | EVQLVESGGG QRNKANDFSI DSGRWGLDAW |
| 21 | hu.1B2.v2 VH | EVQLVESGGG QRNKANDFSI DGGRWGLDAW |
| 22 | hu.1B2.v4 VH | EVQLVESGGG QRNKANDFSI DGGRLALDAW |
| 23 | hu.1B2.v8 VH | EVQLVESGGG QRNKANDFSI DGGKLALDAW |
| 24 | hu.1B2.v9 VH | EVQLVESGGG QRNKANDFSI DGGKWGLDAW |
| 25 | hu.1B2.v101 VL | DIQMTQSPSS ASRLQDGVPS GTKVEIK |
| 26 | hu.1B2.v101 VH | EVQLVESGGG QRNKANDFSI DGGRWGLDAW |
| 27 | hu.1B2.v102 VL | DIQMTQSPSS |
| | | ASRLQDGVPS GTKVEIK |
| 28 | hu.1B2.v102 VH | EVQLVESGGG QRNKANDFSI DGGRLALDAW |
| 29 | hu.1B2.v103 VL | DIQMTQSPSS ASRLQDGVPS GTKVEIK |
| 30 | hu.1B2.v103 VH | EVQLVESGGG QRNKANDFSI DGGKWGLDAW |
| 31 | hu.1B2.v104 VL | DIQMTQSPSS ASRLQDGVPS GTKVEIK |
| 32 | hu.1B2.v104 VH | EVQLVESGGG QRNKANDFSI DGGKLALDAW |
| 33 | rat.3107 CDR-L1, hu.3107.L1, hu.3107.L6, and hu.3107.L7 | RSSQSLVHSDGNTYLH |
| 34 | rat.3107 CDR-L2, hu.3107.L1, hu.3107.L6, and hu.3107.L7 | RISNRFS |
| 35 | rat.3107 CDR-L3, hu.3107.L1, hu.3107.L6, and hu.3107.L7 | LQSTHFPDT |
| 36 | rat.3107 CDR-H1, hu.3107.V1-2.H1, hu.3107.H12, hu.3107.H13, hu.3107.V5-51.H1, hu.3107.H14, and hu.3107.H15 | NYVIH |
| 37 | rat.3107 CDR-H2, hu.3107.V1-2.H1, hu.3107.H12, hu.3107.H13, hu.3107.V5-51.H1, hu.3107.H14, and hu.3107.H15 | YIIPGSGGTKFNEKFKG |
| 38 | rat.3107 CDR-H3, hu.3107.V1-2.H1, hu.3107.H12, hu.3107.H13, hu.3107.V5-51.H1, hu.3107.H14, and hu.3107.H15 | DGAGSFTY |
| 39 | rat.3107 VL | DVLMTQTPVS LLIYRISNRF DTFGGGTKVE |
| 40 | rat.3107 VH | QVQLQQSGAE IIPGSGGTKF AGSFTYWGQG |
| 98 | hu.3107.L1 VL | DVVMTQSPLS LLIYRISNRF DTFGGGTKVE |
| 99 | hu.3107.L6 VL | DVVMTQSPDS LLIYRISNRF DTFGGGTKVE |
| 100 | hu.3107.L7 VL | DIVMTQSPDS LLIYRISNRF DTFGGGTKVE |
| 101 | hu.3107.V1-2.H1 VH | EVQLVQSGAE IIPGSGGTKF AGSFTYWGQG |
| 102 | hu.3107.H12 VH | EVQLVQSGAE IIPGSGGTKF AGSFTYWGQG |
| 103 | hu.3107.H13 VH | EVQLVQSGAE IIPGSGGTKF AGSFTYWGQG |
| 104 | hu.3107.V5-51.H1 VH | EVQLVQSGAE IIPGSGGTKF AGSFTYWGQG |
| 105 | hu.3107.H14 VH | EVQLVQSGAE IIPGSGGTKF AGSFTYWGQG |
| 106 | hu.3107.H15 VH | EVQLVQSGAE IIPGSGGTKF AGSFTYWGQG |
| 41 | rb.2338 CDR-L1 | QASQSISSYLA |
| 42 | rb.2338 CDR-L2 | RASKLAS |
| 43 | rb.2338 CDR-L3 | QSNSYGNNWVGG |
| 44 | rb.2338 CDR-H1 | SGYDMC |
| 45 | rb.2338 CDR-H2 | CIYAGSEGFTYYASWAK |
| 46 | rb.2338 CDR-H3 | WTDSDGSNL |
| 47 | rb.2338 VL | DVVMTQTPAS ASKLASGVPS FGGGTKVEIK |
| 48 | rb.2338 VH | QSLEESGGDL IYAGSEGFTY DSDGSNLWGP |
| 49 | rb.2430, rb.2430.C95dS CDR-L1 | QASQSVVNNRLA |
| 50 | rb.2430, rb.2430.C95dS CDR-L2 | GASTLES |
| 51 | rb.2430 CDR-L3 | QGEFLCSSGDCVA |
| 52 | rb.2430.C95dS CDR-L3 | QGEFLCSSGDSVA |
| 53 | rb.2430, rb.2430.C95dS CDR-H1 | SYDMS |
| 54 | rb.2430, rb.2430.C95dS CDR-H2 | IIQAGSNTLFYASWA |
| 55 | rb.2430, rb.2430.C95dS CDR-H3 | GGVIFIIGHFNL |
| 56 | rb.2430 VL | AQVLTQTASS GASTLESGVS VAFGGGTKVE |
| 57 | rb.2430.C95dS VL | AQVLTQTASS GASTLESGVS VAFGGGTKVE |
| 58 | rb.2430 VH | QSVEESGGRL QAGSNTLFYA IIGHFNLWGP |
| 59 | rb.2621 CDR-L1 | QASESIGSYLA |
| 60 | rb.2621 CDR-L2 | RASTLAS |
| 61 | rb.2621 CDR-L3 | QQTYSGAGVDNL |
| 62 | rb.2621 CDR-H1 | SGYDMC |
| 63 | rb.2621 CDR-H2 | CIVTVSGNTYYASWAK |
| 64 | rb.2621 CDR-H3 | DGGFTDTWYFHL |
| 65 | rb.2621 VL | AYDMTQTPAS ASTLASGVPS FGGGTKVEIK |
| 66 | rb.2621 VH | QSLEESGGGL IVTVSGNTYY FTDTWYFHLW |
| 67 | MBP-huNotch2 EGF6-10 | AGSMGKIEEG KFPQVAATGD AVRYNGKLIA ALMFNLQEPY VDLIKNKHMN VLPTFKGQPS DKPLGAVALK AVINAASGRQ CAPSPCVNGG CVDGVNTYNC VCVNGWSGDD LDDACISNPC HHHHH |
| 68 | MBP-huNotch2 EGF7-9 | AGSMGKIEEG KFPQVAATGD AVRYNGKLIA ALMFNLQEPY VDLIKNKHMN VLPTFKGQPS DKPLGAVALK AVINAASGRQ CPNHRCQNGG TCANRNGGYG CPEGKAGLLC |
| 69 | huNotch2 EGF7 (amino acids 260-296); cynoNotch2 EGF7 (amino acids 260-296) guinea pig Notch2 EGF7 (amino acids 244-280) | N IDDCPNHRCQ NGGVCVDGVN TYNCRCPPQW TGQFCT |
| 70 | Human Notch2, with signal sequence (amino acids 1-25) | MPALRPALLW CKCPEGFLGE CQYSTSHPCF HPCANGSTCT LPGSYQCQCP GFEGSTCERN ECLLQPNACQ STCIDRVASF CTCPQGYKGA PRCEMDINEC NPCVNNGQCV PNGYECQCAT GAICSDQIDE SNPCIHGICM VNGFRCICPE GINCEVDKNE SNPCLNQGTC SPNFESYTCL PGFSGMDCEE NECLSEPCKN TCVDGINSFS |
| | | CPLGYTGKNC PNVSCDIAAS DECASNPCQH TCIDLVNHFK RCLPGFAGER CETFVDVCPQ CRKGEQCVHT QCAPPFSGSR DGGDCSLTME KTCKYDKYCA VLMPPEQLLQ KKQRMTRRSL ASHAIQGTLS KRKRKHGSLW GTGTSEHWVD RTPSLALTPP AEDSSANIIT ADANAQDNMG AARLAVEGMV ANRDMQDNKE RDRMHHDIVR KKSRRPSAKS VDSLESPHTY FSNLHEMQPL ADWMNRMEVN LPPIVTFQLI VAFPTAMMPQ TPSHSGHLQG GQRGPGTHMS |
| 71 | Cynomolgus monkey Notch2, with signal sequence (amino acids 1-25 | M PALRPALLWA LLALWLCRAA PARALQCRDG YEPCVNEGMC |
| | | VTYHNGTGYC CASGFTGEDC CQWTDACLSH CQHGGTCLNL FTFECNCLPG GQFCTEDVDE CAFASCTPGS TNPLNGQYIC CECLKGYAGP ELEINECQSN LNGAKCIDHP TCICNPGYMG CEINFDDCAS RKGATCINGV KCLCDAGWVG CQVNIDECAS CENAAVCKES QGSYMCECPP TGDKCQTDMN TESSCFNGGT DGLGTYHCSC GWAGAYCDVP TGSYCEEQLD QNQPCQNGGT VDRIGGYSCR CRSAFTGRHC CQSSCGQVKC QRQPPYYSCQ ACNSHACQWD DNFECQGNSK LAEGTLVIVV YYGEKSAAMK NTDAAAALLA IILLGVIMAK |
| | | VQVSEANLIG QHLEAADIRR SDLSDEDEDA AAKRLLDAGA NDGTTPLILA ATLLLLKNGA MDRLPRDVAR LSLKHTPMGK SESSVTLSPV SVHAQHALSF SRLHPVPVPA KHITTPREPL IPEMARLPSV YASSNAAERT SPTPGGAGGG |
| 72 | Guinea pig Notch2, with signal sequence (amino acids 1-9) | MYLFCFVLAL PCEKNRCQNG NGGTCHMLSW SCKCLAGFTG HCDNPYVPCA HRCQNGGVCV NRNGGYGCVC GKAGLLCHLD DECAMTNSNP NDATCLDKIG QCLCPPGFTG CEENIDNCDP LNEGRCIDLV SCVCSPGFTG CYSQVNECLS QNGGTCDNLV YTCQCALPYT GQRCTVDIDE ANPCQNGGSC YVNSYTCKCQ FTGPFCLHEI CSQSPCKNKG KNLCKNSGAC FIGGYRCECV TRGLFCEENI ECLSNPCNSE GTCAVASNMP FCPSPQDCES PTSIPSDICA SSPLPCWNYI HCDQGCNSEE RALGTLLHTN EVAGSQVFLE VSESLSPKPT LRRDSSNHKR KKAKAEDEAL DVLDVNVRGP ASLQAQTDRT AVAADAQGVF QADVNAVDDH AAREGSYEAA VTPSPPGTVL PNLAKEAKDA SPMITSPGIL VLPSVSQLLS MFGMVLTPAE QPAGASQPQS QTILPAYHPF PSPDSPDQWS |
| | | MQVYA |
| 73 | Mouse Notch2, with signal sequence (amino acids 1-25) | MPALRPAALR CRCPEGFLGE CQYSTSHPCF HPCENGSTCT LPGSYRCQCP GFEGSTCERN ECLLQPNACQ STCIDRVASF CTCPQGYKGA PRCEMDINEC NPCVNNGQCV PNGYECQCAT GAICSDQIDE SNPCMHGVCV VNGFRCICPE GVNCEVDKNE SNPCLNQGTC APNFESFSCL PGFSGMDCEE NECLSEPCKN TCVDGINSFS CPLGYTGKNC LNVSCKAAAL DECASNPCQH TCIDLVNHFK RCLPGFAGER CETFLDVCPQ CRRGEQCIHT RCPPSFGGSH DGGDCSLTME KTCKYDKYCA VLLPPEQLLQ KKQKMTRRSL LLASHAIQGT MAKRKRKHGF LIGSGTSEHW RHTPSLALTP DAEDSSANII GADANAQDNM LAARLAVEGM GANRDMQDNK ARDRMHHDIV GKKARRPNTK PVDSLESPHT SFSNLHDMQP PADWMNRVEM EPLPPIVTFQ PSVAFPPTMM ERTPSHGGHL GGGQRGPGTH |
| 74 | huNotch2-EGF6-10 | AGSDSLYVPC NHRCQNGGVC ANRNGGYGCV EGKAGLLCHL VDEGNSHHHH |
| 75 | muNotch2-EGF6-10 | AGSDSPYVPC NHKCQNGGVC TNRNGGYGCV EGKAGLLCHL VDEGNSGLND |
| 76 | gpNotch2-EGF6-12 | AGSDNPYVPC NHRCQNGGVC |
| | | TNRNGGYGCV EGKAGLLCHL VDECAMTNSN QNDATCLDKI NLYFQGHHHH |
| 77 | huNotch2-EGF6-12.R268K | AGSDSLYVPC NHKCQNGGVC ANRNGGYGCV EGKAGLLCHL VDECAMANSN QNDATCLDKI GHHHHHHHH |
| 78 | muNotch2-EGF6-12.K268R | AGSDSPYVPC NHRCQNGGVC TNRNGGYGCV EGKAGLLCHL VDECAMANSN QNDATCLDKI NLYFQGHHHH |
| 79 | ratNotch2-EGF6-10 | AGSDSPYVPC NHKCQNGGVC TNRNGGYGCV EGKAGLLCHL VDEGNSGLND |
| 80 | muNotch2 EGF7 (amino acids 260-296) | N IDDCPNHKCQ NGGVCVDGVN TYNCRCPPQW TGQFCT |
| 81 | Rat Notch2, with signal sequence (amino acids 1-25) | MPALRPAALR CRCPEGFLGE CQYSTSHPCF HPCENGSTCS LPGSYRCQCP GFEGSNCERN ECLLQPNACQ STCIDRVASF CTCPQAYKGA PRCEMDINEC NPCVNNGQCV PNGYECQCAT GAICSDQIDE SNPCLHGACV VNGFRCMCPE GINCEVDKNE SNPCLNQGTC APNFESFTCL PGFSGMDCEE NECLSEPCKN TCVDGINSFS CPLGYTGKNC LNVSCKAAAL DECASNPCQH TCIDLVNHFK RCLPGFAGER CETFLDVCPQ CRRGEQCVHT RCSPPFWGSH DGGDCSLTME KTCKYDKYCA VLLPPEQLLQ KKQKVARRSL ASHAIQGTLS KRKRKHGFLW GSTTSEHWGD RTPSLALTPP |
| | | AEDSSANIIT ADANAQDNMG AARLAVEGMV ANRDMQDNKE RDRMHHDIVR KKARRPNTKS VDSLESPHTY SNLHEMQPLR DWMNRVEMSE LPPIVTFQLI VAFPPTMMPQ TPNHGGHLQG GQRGPGTHMS |
| 82 | huNotch2-EGF4-7 | GSQWTDACLS HCQHGGTCLN DFTFECNCLP TGQFCTEDVD |
| 83 | huNotch2-EGF5-8 | GSETDVNECD SPCVNGGTCR GVNTYNCRCP NGWSGDDCSE |
| 84 | huNotch2-EGF7-9 | AHHHAHHHAGEN GQFCTEDVDE CAFASCTPGS |
| 85 | huNotch1 | AGSERPYVPC GNNCKNGGAC HNTHGGYNCV HGRTGLLCHL VDEGNSGLND |
| 86 | huNotch3 | GSENPAVPCA HRCLNGGTCV NTLGGHSCVC GKTGLLCHLD DECSIGANPC QATCLDRIGQ YFQGHHHHHH |

What is claimed is:

1. An isolated antibody that binds to human Notch2, wherein the antibody inhibits Jagged1-mediated signaling, but does not inhibit DLL1-mediated signaling.

2. An isolated antibody that binds to human Notch2, wherein the antibody inhibits Jagged1-mediated signaling to a greater extent than DLL1-mediated signaling.

3. The isolated antibody of claim 2, wherein the antibody is capable of achieving a maximum inhibition of Jagged1-mediated signaling of 100%, and a maximum inhibition of DLL1-mediated signaling of less than 80%, or less than 70%, or less than 60%.

4. The isolated antibody of claim 2 or claim 3, wherein the antibody is a Fab fragment.

5. The isolated antibody of claim 4, wherein the antibody, when formatted as a bivalent IgG antibody comprising two heavy chains and two light chains, inhibits Jagged1-mediated signaling, but does not inhibit DLL1-mediated signaling.

6. The isolated antibody of any one of claims 1-5, wherein the antibody does not inhibit binding of Jagged1 to Notch2.

7. The isolated antibody of any one of claims 1-6, wherein the antibody does not inhibit binding of DLL1 to Notch2.

8. The isolated antibody of any one of claims 1-7, wherein the antibody binds an epitope within the EGF7 repeat of Notch2.

9. The isolated antibody of any one of claims 1-8, wherein the antibody binds an epitope within amino acids 260-296 of Notch2.

10. The isolated antibody of any one of claims 1-8, wherein the antibody binds a discontinuous epitope within amino acids 260-296 of Notch2.

11. An isolated antibody that binds to Notch2, wherein the antibody binds an epitope within the EGF7 repeat of Notch2.

12. An isolated antibody that binds to Notch2, wherein the antibody binds an epitope within amino acids 260-296 of Notch2.

13. An isolated antibody that binds to Notch2, wherein the antibody binds a discontinuous epitope within amino acids 260-296 of Notch2.

14. The isolated antibody of any one of claims 1-13, wherein the antibody contacts arginine 268 (R268) of human Notch2.

15. The isolated antibody of claim 14, wherein the antibody does not bind a Notch2 comprising lysine 268 (K268).

16. The isolated antibody of any one of claims 1-15, wherein the antibody binds a polypeptide comprising the amino acid sequence of SEQ ID NO: 74 and does not bind a polypeptide comprising the amino acid sequence of SEQ ID NO: 77.

17. The isolated antibody any one of claims 1-16, wherein the antibody binds to human Notch2 and cynomolgus monkey Notch2.

18. The isolated antibody of any one of claims 1-17, wherein the antibody does not bind to mouse Notch2.

19. The isolated antibody of any one of claims 1-18, wherein the antibody binds to guinea pig Notch2.

20. The isolated antibody of any one of claims 1-19, wherein the antibody does not bind to human Notch1 or human Notch3.

21. The isolated antibody of any one of claims 1-20, wherein the antibody binds human Notch2 with an affinity (K_{D}) of less than 20 nM, less than 15 nM, less than 10 nM, or less than 5 nM, as determined by surface plasmon resonance.

22. The isolated antibody of any one of claims 1-21, wherein the antibody inhibits Jagged1-mediated signaling with an IC50 of less than 20 nM, less than 15 nM, less than 10 nM, or less than 5 nM.

23. The isolated antibody of claim 22, wherein inhibition of Jagged1-mediated signaling is determined using a high-content screening (HCS) assay.

24. The isolated antibody of any one of claims 1-23, wherein the antibody comprises:
a) a heavy chain variable domain (VH) comprising (a) CDR-H1 comprising the amino acid sequence of SEQ ID NO: 4, (b) CDR-H2 comprising the amino acid sequence of SEQ ID NO: 6 or 7, and (c) CDR-H3 comprising the amino acid sequence of SEQ ID NO: 8, 9, 10, 11, or 12, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3;
b) a heavy chain variable domain (VH) comprising (a) CDR-H1 comprising the amino acid sequence of SEQ ID NO: 36, (b) CDR-H2 comprising the amino acid sequence of SEQ ID NO: 37, and (c) CDR-H3 comprising the amino acid sequence of SEQ ID NO: 38, and a light chain variable domain (VL) comprising (d) CDR-L1 comprising the amino acid sequence of SEQ ID NO: 33, (e) CDR-L2 comprising the amino acid sequence of SEQ ID NO: 34, and (f) CDR-L3 comprising the amino acid sequence of SEQ ID NO: 35;
c) a heavy chain variable domain (VH) comprising (a) CDR-H1 comprising the amino acid sequence of SEQ ID NO: 44, (b) CDR-H2 comprising the amino acid sequence of SEQ ID NO: 45, and (c) CDR-H3 comprising the amino acid sequence of SEQ ID NO: 46, and a light chain variable domain (VL) comprising (d) CDR-L1 comprising the amino acid sequence of SEQ ID NO: 41, (e) CDR-L2 comprising the amino acid sequence of SEQ ID NO: 42, and (f) CDR-L3 comprising the amino acid sequence of SEQ ID NO: 43;
d) a heavy chain variable domain (VH) comprising (a) CDR-H1 comprising the amino acid sequence of SEQ ID NO: 53, (b) CDR-H2 comprising the amino acid sequence of SEQ ID NO: 54, and (c) CDR-H3 comprising the amino acid sequence of SEQ ID NO: 55, and a light chain variable domain (VL) comprising (d) CDR-L1 comprising the amino acid sequence of SEQ ID NO: 49, (e) CDR-L2 comprising the amino acid sequence of SEQ ID NO: 50, and (f) CDR-L3 comprising the amino acid sequence of SEQ ID NO: 51 or 52; or
e) a heavy chain variable domain (VH) comprising (a) CDR-H1 comprising the amino acid sequence of SEQ ID NO: 62, (b) CDR-H2 comprising the amino acid sequence of SEQ ID NO: 63, and (c) CDR-H3 comprising the amino acid sequence of SEQ ID NO: 64, and a light chain variable domain (VL) comprising (d) CDR-L1 comprising the amino acid sequence of SEQ ID NO: 59, (e) CDR-L2 comprising the amino acid sequence of SEQ ID NO: 60, and (f) CDR-L3 comprising the amino acid sequence of SEQ ID NO: 61.

25. An isolated antibody that binds to human Notch2, wherein the antibody comprises:
a) a heavy chain variable domain (VH) comprising (a) CDR-H1 comprising the amino acid sequence of SEQ ID NO: 4, (b) CDR-H2 comprising the amino acid sequence of SEQ ID NO: 6 or 7, and (c) CDR-H3 comprising the amino acid sequence of SEQ ID NO: 8, 9, 10, 11, or 12, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3;
b) a heavy chain variable domain (VH) comprising (a) CDR-H1 comprising the amino acid sequence of SEQ ID NO: 36, (b) CDR-H2 comprising the amino acid sequence of SEQ ID NO: 37, and (c) CDR-H3 comprising the amino acid sequence of SEQ ID NO: 38, and a light chain variable domain (VL) comprising (d) CDR-L1 comprising the amino acid sequence of SEQ ID NO: 33, (e) CDR-L2 comprising the amino acid sequence of SEQ ID NO: 34, and (f) CDR-L3 comprising the amino acid sequence of SEQ ID NO: 35;
c) a heavy chain variable domain (VH) comprising (a) CDR-H1 comprising the amino acid sequence of SEQ ID NO: 44, (b) CDR-H2 comprising the amino acid sequence of SEQ ID NO: 45, and (c) CDR-H3 comprising the amino acid sequence of SEQ ID NO: 46, and a light chain variable domain (VL) comprising (d) CDR-L1 comprising the amino acid sequence of SEQ ID NO: 41, (e) CDR-L2 comprising the amino acid sequence of SEQ ID NO: 42, and (f) CDR-L3 comprising the amino acid sequence of SEQ ID NO: 43;
d) a heavy chain variable domain (VH) comprising (a) CDR-H1 comprising the amino acid sequence of SEQ ID NO: 53, (b) CDR-H2 comprising the amino acid sequence of SEQ ID NO: 54, and (c) CDR-H3 comprising the amino acid sequence of SEQ ID NO: 55, and a light chain variable domain (VL) comprising (d) CDR-L1 comprising the amino acid sequence of SEQ ID NO: 49, (e) CDR-L2 comprising the amino acid sequence of SEQ ID NO: 50, and (f) CDR-L3 comprising the amino acid sequence of SEQ ID NO: 51 or 52; or
e) a heavy chain variable domain (VH) comprising (a) CDR-H1 comprising the amino acid sequence of SEQ ID NO: 62, (b) CDR-H2 comprising the amino acid sequence of SEQ ID NO: 63, and (c) CDR-H3 comprising the amino acid sequence of SEQ ID NO: 64, and a light chain variable domain (VL) comprising (d) CDR-L1 comprising the amino acid sequence of SEQ ID NO: 59, (e) CDR-L2 comprising the amino acid sequence of SEQ ID NO: 60, and (f) CDR-L3 comprising the amino acid sequence of SEQ ID NO: 61.

26. The isolated antibody of any one of claims 1-25, wherein the antibody comprises:
a) a VH sequence having at least 95% sequence identity to the amino acid sequence of SEQ ID NO: 14;
b) a VL sequence having at least 95% sequence identity to the amino acid sequence of SEQ ID NO: 13;
c) a VH sequence as defined in (a) and a VL sequence as defined in (b);
d) a VH sequence having at least 95% sequence identity to an amino acid sequence selected from SEQ ID NOs: 17-24, 26, 28, 30, and 32;
e) a VL sequence having at least 95% sequence identity to an amino acid sequence selected from SEQ ID NOs: 15, 16, 25, 27, 29, and 31;
f) a VH sequence as defined in (d) and a VL sequence as defined in (e);
g) a VH sequence having at least 95% sequence identity to the amino acid sequence of SEQ ID NO: 40;
h) a VL sequence having at least 95% sequence identity to the amino acid sequence of SEQ ID NO: 39;
i) a VH sequence as defined in (g) and a VL sequence as defined in (h);
j) a VH sequence having at least 95% sequence identity to an amino acid sequence selected from SEQ ID NOs: 102-106;
k) a VL sequence having at least 95% sequence identity to an amino acid sequence selected from SEQ ID NOs: 98-100;
l) a VH sequence as defined in (j) and a VL sequence as defined in (k);
m) a VH sequence having at least 95% sequence identity to the amino acid sequence of SEQ ID NO: 48;
n) a VL sequence having at least 95% sequence identity to the amino acid sequence of SEQ ID NO: 47;
o) a VH sequence as defined in (m) and a VL sequence as defined in (n);
p) a VH sequence having at least 95% sequence identity to the amino acid sequence of SEQ ID NO: 58;
q) a VL sequence having at least 95% sequence identity to the amino acid sequence of SEQ ID NO: 56 or 57;
r) a VH sequence as defined in (p) and a VL sequence as defined in (q);
s) a VH sequence having at least 95% sequence identity to the amino acid sequence of SEQ ID NO: 66;
t) a VL sequence having at least 95% sequence identity to the amino acid sequence of SEQ ID NO: 65; or
u) a VH sequence as defined in (s) and a VL sequence as defined in (t).

27. The isolated antibody of any one of claims 1-26, wherein the antibody comprises:
a) a VH sequence comprising the amino acid sequence of SEQ ID NO: 14;
b) a VL sequence comprising the amino acid sequence of SEQ ID NO: 13;
c) a VH sequence as defined in (a) and a VL sequence as defined in (b);
d) a VH sequence comprising an amino acid sequence selected from SEQ ID NOs: 17-24, 26, 28, 30, and 32;
e) a VL sequence comprising an amino acid sequence selected from SEQ ID NOs: 15, 16, 25, 27, 29, and 31;
f) a VH sequence as defined in (d) and a VL sequence as defined in (e);
g) a VH sequence comprising the amino acid sequence of SEQ ID NO: 40;
h) a VL sequence comprising the amino acid sequence of SEQ ID NO: 39;
i) a VH sequence as defined in (g) and a VL sequence as defined in (h);
j) a VH sequence comprising an amino acid sequence selected from SEQ ID NOs: 101-106;
k) a VL sequence comprising an amino acid sequence selected from SEQ ID NOs: 98-100;
l) a VH sequence as defined in (j) and a VL sequence as defined in (k);
m) a VH sequence comprising the amino acid sequence of SEQ ID NO: 48;
n) a VL sequence comprising the amino acid sequence of SEQ ID NO: 47;
o) a VH sequence as defined in (m) and a VL sequence as defined in (n);
p) a VH sequence comprising the amino acid sequence of SEQ ID NO: 58;
q) a VL sequence comprising the amino acid sequence of SEQ ID NO: 56 or 57;
r) a VH sequence as defined in (p) and a VL sequence as defined in (q);
s) a VH sequence comprising the amino acid sequence of SEQ ID NO: 66;
t) a VL sequence comprising the amino acid sequence of SEQ ID NO: 65; or
u) a VH sequence as defined in (s) and a VL sequence as defined in (t).

28. An isolated antibody that binds to human Notch2, wherein the antibody comprises:
a) a VH sequence comprising the amino acid sequence of SEQ ID NO: 14;
b) a VL sequence comprising the amino acid sequence of SEQ ID NO: 13;
c) a VH sequence as defined in (a) and a VL sequence as defined in (b);
d) a VH sequence comprising an amino acid sequence selected from SEQ ID NOs: 17-24, 26, 28, 30, and 32;
e) a VL sequence comprising an amino acid sequence selected from SEQ ID NOs: 15, 16, 25, 27, 29, and 31;
f) a VH sequence as defined in (d) and a VL sequence as defined in (e);
g) a VH sequence comprising the amino acid sequence of SEQ ID NO: 40;
h) a VL sequence comprising the amino acid sequence of SEQ ID NO: 39;
i) a VH sequence as defined in (g) and a VL sequence as defined in (h);
j) a VH sequence comprising an amino acid sequence selected from SEQ ID NOs: 101-106;
k) a VL sequence comprising an amino acid sequence selected from SEQ ID NOs: 98-100;
l) a VH sequence as defined in (j) and a VL sequence as defined in (k);
m) a VH sequence comprising the amino acid sequence of SEQ ID NO: 48;
n) a VL sequence comprising the amino acid sequence of SEQ ID NO: 47;
o) a VH sequence as defined in (m) and a VL sequence as defined in (n);
p) a VH sequence comprising the amino acid sequence of SEQ ID NO: 58;
q) a VL sequence comprising the amino acid sequence of SEQ ID NO: 56 or 57;
r) a VH sequence as defined in (p) and a VL sequence as defined in (q);
s) a VH sequence comprising the amino acid sequence of SEQ ID NO: 66;
t) a VL sequence comprising the amino acid sequence of SEQ ID NO: 65; or
u) a VH sequence as defined in (s) and a VL sequence as defined in (t).

29. The isolated antibody of any one of claims 1-25, wherein the antibody comprises:
a) a VH sequence having at least 95% sequence identity to an amino acid sequence selected from SEQ ID NOs: 17-24, 26, 28, 30, and 32;
b) a VL sequence having at least 95% sequence identity to an amino acid sequence selected from SEQ ID NOs: 15, 16, 25, 27, 29, and 31;
c) a VH sequence as defined in (a) and a VL sequence as defined in (b);
d) a VH sequence having at least 95% sequence identity to an amino acid sequence selected from SEQ ID NOs: 102-106;
e) a VL sequence having at least 95% sequence identity to an amino acid sequence selected from SEQ ID NOs: 98-100; or
f) a VH sequence as defined in (d) and a VL sequence as defined in (e).

30. The antibody of any one of claims 1-25 and 29, wherein the antibody comprises:
a) a VH sequence comprising an amino acid sequence selected from SEQ ID NOs: 17-24, 26, 28, 30, and 32;
b) a VL sequence comprising an amino acid sequence selected from SEQ ID NOs: 15, 16, 25, 27, 29, and 31;
c) a VH sequence as defined in (a) and a VL sequence as defined in (b);
d) a VH sequence comprising an amino acid sequence selected from SEQ ID NOs: 101-106;
e) a VL sequence comprising an amino acid sequence selected from SEQ ID NOs: 98-100; or
f) a VH sequence as defined in (d) and a VL sequence as defined in (e).

31. The isolated antibody of any one of claims 1-25, wherein the antibody:
a) comprises a VH sequence of SEQ ID NO: 26 and a VL sequence of SEQ ID NO: 25;
b) comprises a VH sequence of SEQ ID NO: 28 and a VL sequence of SEQ ID NO: 27;
c) comprises a VH sequence of SEQ ID NO: 30 and a VL sequence of SEQ ID NO: 29; or
d) comprises a VH sequence of SEQ ID NO: 32 and a VL sequence of SEQ ID NO: 31.

32. An isolated antibody that binds to human Notch2, wherein the antibody:
a) comprises a VH sequence of SEQ ID NO: 26 and a VL sequence of SEQ ID NO: 25;
b) comprises a VH sequence of SEQ ID NO: 28 and a VL sequence of SEQ ID NO: 27;
c) comprises a VH sequence of SEQ ID NO: 30 and a VL sequence of SEQ ID NO: 29; or
d) comprises a VH sequence of SEQ ID NO: 32 and a VL sequence of SEQ ID NO: 31.

33. The isolated antibody of any one of claims 1-32, which is a monoclonal antibody.

34. The isolated antibody of any one of claims 1-33, which is a human, humanized, or chimeric antibody.

35. The isolated antibody of any one of claims 1-34, which is an antibody fragment that binds Notch2.

36. The isolated antibody of claim 35, wherein the antibody fragment is selected from Fv, Fab, Fab′, Fab′-SH, and F(ab′)₂.

37. The isolated antibody of claim 36, wherein the antibody fragment is a Fab, Fab′, or Fab′-SH.

38. The isolated antibody of any of claims 1-3 and 6-37, which is a full-length antibody.

39. The isolated antibody of claim 38, wherein the antibody is a full-length IgG antibody.

40. The isolated antibody of claim 39, wherein the antibody is an IgG1, IgG2, IgG3, or IgG4 antibody.

41. An isolated antibody that competes for binding to human Notch2 with the antibody of any one of claims 1-40.

42. An isolated nucleic acid encoding the antibody of any of claims 1-41.

43. A host cell comprising the nucleic acid of claim 42.

44. A host cell that expresses the antibody of any one of claims 1-41.

45. A method of producing an antibody that binds to human Notch2 comprising culturing the host cell of claim 43 or claim 44 under conditions suitable for the expression of the antibody.

46. The method of claim 45, further comprising recovering the antibody from the host cell.

47. An antibody produced by the method of claim 45 or claim 46.

48. A pharmaceutical composition comprising the antibody of any of claims 1-41 and a pharmaceutically acceptable carrier.

49. The pharmaceutical composition of claim 48, further comprising an additional therapeutic agent.

50. The pharmaceutical composition of claim 49, wherein the additional therapeutic agent is selected from hypertonic saline, mannitol, pulmozyme, N-acetyl cysteine, cysteamine, and a bronchodilator.

51. The antibody of any one of claims 1-41 or the pharmaceutical composition of any one of claims 48-50 for use as a medicament.

52. The antibody of any one of claims 1-41 or the pharmaceutical composition of any one of claims 48-50 for use in treating a muco-obstructive lung disease.

53. The antibody for use of claim 52, wherein the muco-obstructive lung disease is selected from chronic obstructive lung disease (COPD), cystic fibrosis, primary ciliary dyskinesia, non-cystic fibrosis bronchiectasis, and bronchiolitis.

54. Use of the antibody of any one of claims 1-41 or the pharmaceutical composition of any one of claims 48-50 in the manufacture of a medicament for treating a muco-obstructive lung disease.

55. The use of claim 54, wherein the muco-obstructive lung disease is selected from chronic obstructive lung disease (COPD), cystic fibrosis, primary ciliary dyskinesia, non-cystic fibrosis bronchiectasis, and bronchiolitis.

56. Use of the antibody of any one of claims 1-41 or the pharmaceutical composition of any one of claims 48-50 in the manufacture of a medicament for reducing the number of secretory cells in a subject.

57. The use of claim 56, the medicament converts secretory cells to ciliated cells.

58. The use of claim 56 or claim 57, wherein the secretory cells are in the lungs of the subj ect.

59. The use of any one of claims 56-58, wherein the secretory cells are goblet cells.

60. A method of treating subject with a muco-obstructive lung disease, comprising administering to the subject an effective amount of the antibody of any one of claims 1-41 or the pharmaceutical composition of any one of claims 48-50.

61. The method of claim 60, wherein the muco-obstructive lung disease is selected from chronic obstructive lung disease (COPD), cystic fibrosis, primary ciliary dyskinesia, non-cystic fibrosis bronchiectasis, and bronchiolitis.

62. A method of reducing the number of secretory cells in a subject, comprising administering to the individual an effective amount of the antibody of any of claims 1-41 or the pharmaceutical composition of any one of claims 48-50 to deplete secretory cells in the subject.

63. The method of claim 62, wherein the method comprises converting secretory cells to ciliated cells.

64. The method of claim 62 or claim 63, wherein the secretory cells are in the lungs of the subject.

65. The method of any one of claims 62-64, wherein the secretory cells are goblet cells.

66. The method of any one of claims 60-65, further comprising administering an additional therapeutic agent to the subject.

67. The method of claim 66, wherein the additional therapeutic agent is selected from hypertonic saline, mannitol, pulmozyme, N-acetyl cysteine, cysteamine, and a bronchodilator.
